(19) United States
(12) Patent Application Publication
(10) Pub. No.: US 2011/0213236 A1
(43) Pub. Date: Sep. 1, 2011

(54) THERAPEUTIC COMPOSITIONS, DEVICES AND METHODS FOR OBSERVING TREATED TISSUES

(75) Inventor: LEONARD S. GIRSH, Naples, FL (US)

(73) Assignee: IMMUNOPATH PROFILE, INC., Naples, FL (US)

(21) Appl. No.: 13/021,487

(22) Filed: Feb. 4, 2011

Related U.S. Application Data
(63) Continuation-in-part of application No. PCT/US2009/053021, filed on Aug. 6, 2009.
(60) Provisional application No. 61/086,574, filed on Aug. 6, 2008.
(60) Provisional application No. 61/301,404, filed on Feb. 4, 2010.
(60) Provisional application No. 61/389,977, filed on Oct. 5, 2010.

(51) Int. Cl. A61B 5/055; A61B 6/00; A61B 8/00; A61B 6/03; A61K 35/34; A61P 29/00; A61P 37/04

(52) U.S. Cl. 600/410; 600/476; 600/437; 378/4; 424/548

(57) ABSTRACT

The subject invention pertains to a synthetic biopharmaceutical non-invasive medical therapy, administered locally or systemically and compositions for treating aged, diseased or abnormal tissues and organs. The composition and methods of the invention may also be used to augment the treatment of multiple diseases and disorders of the body. The present invention involves administering to a patient a therapeutic formulation comprising a free L-amino acid non-chiral glycine profile, simulating or replicating the proteins normally present in healthy tissue that is now diseased or transplanted tissue. The invention also relates to therapy involving administration of therapeutic formulations comprising free L-amino acids in which the molar ratios of the amino acids correspond to the ratios of components such as amino components in a key cell or molecular embryology systemic as a medication or a biochemical and biophysical medication and essence of cell membrane polar surface active lipid equivalent that is useful for treating a disease. Simultaneous administration of the compositions are able to work synergistically to restructure diseased tissue and organs. Also disclosed are devices and methods for diagnosing diseased or damaged tissue by observance of tissue biomarkers made visible by direct application of polarizing light to a tissue sample. Birefringence light from damaged or diseased tissue can be analyzed and compared with normal tissue birefringence to diagnose diseased or damaged tissue or diagnose a tissue deficiency.

### CROSS-REFERENCE TO RELATED APPLICATIONS

This application is a continuation-in-part of International Patent Application No. PCT/US2009/053021, filed Aug. 6, 2009, which claims the benefit of U.S. Provisional Application Ser. No. 61/086,574, filed Aug. 6, 2008. This application also claims the benefit of U.S. Provisional Application Ser. No. 61/301,404, filed Feb. 4, 2010, and U.S. Provisional Application Ser. No. 61/389,977, filed Oct. 5, 2010, the disclosures of which are hereby incorporated by reference in their entireties, including any figures, tables, or drawings.

### FIELD OF THE INVENTION

The present invention relates to a non-invasive medical therapy, and compositions for avoiding organ transplantation, reducing rejection of transplanted organs, or treating organs under consideration for replacement by transplant, and otherwise treating aged, diseased or abnormal tissues and organs. The composition and methods of the invention may also be used to augment the treatment of multiple diseases and disorders of the body such as and inclusive of dermatological disorders, gastrointestinal disorders, ophthalmic disorders (such as diseased corneas), neurologic disorders, bacterial infections, respiratory disorders, tracheo-bronchial disorders, and various disorders related to aging. The present invention involves administering to a patient a therapeutic formulation comprising a free L-amino acid non-chiral glycine profile, simulating or replicating the proteins normally present in healthy tissue that is now diseased or transplanted tissue. The invention also relates to therapy involving administration of therapeutic formulations comprising free L-amino acids in which the molar ratios of the amino acids correspond to the ratios of components such as amino components in a medication that is useful for treating a disease. By simultaneous administration of the various components of one of the therapeutic formulations, the components are able to work synergistically to restructure diseased tissue and organs.

In mimicking human tissue from a biomolecular standpoint and thereby normalizing the cell cycle from a biomolecular standpoint we are providing the patient with opportunities to achieve disease resolution by using this immunotherapeutic composition free of known undesirable effects, we are therapeutically significantly lessening the morbidity and mortality risk and thereby optimizing disease resolution, resulting in, as observed by the clinician and the veterinarian, “incredible healing”. The co-use of this medicine with significant therapeutics, life-saving and life support systems, such as drugs to acutely treat and resolve cardiac arrhythmia such as auricular fibrillation (atrial) and equally importantly side-effects are minimized such as extreme side-effects such as inability to walk, Parkinson's disease symptoms, and leg pain. It is believed by using these components of composition of matter immunotherapeutic agent (IA) we have normalized neuronal flow and pain management, and by normalizing the neuronal flow, even if it is in the conduction system of the heart we have therapeutically prevented not only to atrial fibrillation, but also the side effects which appear to be disturbance of normal neuronal flow.

This broad-based regenerative medicine stem cell activation healing system based on molecular structures that underlie key cell biology systems inclusive of molecular embryology therefore the broad based efficacy that has been anticipated with the natural embryonic stem cell is being replicated as a asynthetic biopharmaceutical using components equivalent to human tissue that also replace documented deficiencies in human tissue and the cell cycle by providing a composition that mimics human tissue from a biological and cell biology system and cell cycle vantage point without any known undesirable effects

### BACKGROUND

The investigators Teahon K, Smethurst P, Pearson M, Levi A J and Bjarnason I. in “The effect of elemental diet on intestinal permeability and inflammation in Crohn's disease.” (Gastroenterol. 1991; 101: 84-8.)

shows that elemental feeding was equal to steroids, but requires great limitations of administration and are proportionate to its ability to substitute for food, whereas in this subject composition we are able to show with complete substitution for food she was able to avoid flare-ups that required steroids, but for only a short period of time because of the palatability of the elemental feeding such as four weeks. Seidman used a similar approach in reversing the complications of steroids and the failure to thrive and to grow and arrested prepubertal development.

In building on this background we have developed a synthetic biopharmaceutical immunotherapeutic agent with molecules underlying key cell biology systems and particular reference to molecular embryology and in activating the patients own stem cells deactivated by disease.

Teahon also emphasized the avoidance of anti-inflammatory NSAID drugs in the treatment of inflammatory bowel disease and further Goodman and Gillman, 10^{th} ed. emphasized the catabolic effect and interference of healing of steroidal anti-inflammatory drugs. Brachet in molecular embryology provides the basis of the activation of the sleeping egg and we have included that in the conceptually in the activation of the stem cell healing system inactivated by disease in the patient that is failing to heal. (Ref. Monroy's observations reported in classic text of Brachet, J and Alexandre, H Introduction to Molecular Embryology, Springer and Verlag, p. 77, New York, 1986).

Healing overlaps with the inflammatory process and is only for didactic purposes that the two are separated (Rubin, Emanuel. Farber, John L. Pathology, 1^{st} ed. Lippincott, Philadelphia. 1988. “The Extracellular Matrix” p. 68) an essential component of the biopharmaceutical to use this as an anti-inflammatory healing broad-based regenerative medicine treatment. This also emphasizes the importance of the extra cellular matrix and its implications as a biorobotic interdependent signaling system where the “matrix contains information which can direct migration, attachment, differentiation, and organization of the cells. The important of the ECM with multi-cellularity is indicated by the production of collagen, laminin, and fibronectin as early cleavage of the fertilized ovum. The information contained in the ECM is important not only for development but also for wound healing. We have built on this information as the algorithm for software.

“The matrix protein of invertebrates, fish, reptiles, and mammals, share a common plan. A third of their amino acid content is glycine and they are rich in the amino acid serine, proline, threonine, and alanine. These four amino acids are coated by RNA triplets with cystosine and uracil as the 2^{nd} and 3^{rd} residue and different from each other only in the first residue of the code. The ECM not only provides tissues with structural support but also exchanges information with cells thereby modulating a host of processes including development, cell migration, attachment, and repair”, essential in wound healing and therefore so included in this subject composition.

However the compositions of the subject invention also contains additional components. To our knowledge, nobody has included in a single treatment plan all of the components described in the orphan drug designation request for the compositions of the subject invention. The recomposition of tissue and make or repair of tissue is analogous to building and or repairing a building, adhering to the templating genetic plan as well as to the cell biology cycle of the cell biology tissue system and the activation of apoptosis as the worn, diseased and damaged tissue, wherein the architectural design of the genetic system and in accord with the cell biology system will not reconstitute the tissue without the essentials of the building blocks of the building architectural genetic plan of tissue recomposition with amino acids as “bricks”, and the essence of cell membrane phospholipid as water, and the extra cellular matrix as steel (as highly sulfated polymer Kevlar, 5 times stronger than steel) hence the analog of building a building where the material is of even greater importance than the architectural genetic plan. Our hypothesis is that the elemental diets discussed in the published literature and described in the designation request were “only” as effective as steroids because the elemental diets were not provided in the optimal molar ratio, and did not include important additional components that are necessary for cellular function and regeneration. In particular, elemental diets lack the components of the cell membrane and extracellular matrix that are provided in the compositions of the subject invention. By supplying the body with the chemical components of the extracellular matrix, such as cartilage containing chondroitin sulfate and collagen, tissue repair and anti-inflammatory activity is enhanced since the extracellular matrix provides tissues with structural support and directs cell migration, attachment, and organization. Cell membrane formation and repair is enhanced by the simultaneous administration of phospholipids, essential fatty acids, and EPA. In sum, the compositions of the subject invention are designed to repair diseased human tissue and enhance the formation of healthy tissue. Since all of the components are necessary for cellular function, all of the moieties in the compositions of the subject invention are thought to be essential and “active.” We discussed these other moieties in the designation request, and provided references for many of the components that are not already part of the elemental diets described above. Additional information and references can be found throughout the relevant literature. And further includes the activation of apoptotic enzymatic lysosomal system dependent not only on highly hydrophilic activity and associated hydrogen bonding I augmenting surface-surface interaction of substrate and enzyme but also in the activation of templating as seen in molecular embryology in activation of the sleeping egg, where the enzyme activation in the phospholipase in the head of the sperm breaks off the arm of the phospholipid lyso phosphatidyl choline and becomes much more hydrophilically active surfactant in activating ribosomal templating as well as the proteomic display of apoptosis. The further activation of the cell cycle system by activating the mitochondrial bioenergy system in providing energy for healing as well the cell cycle assembly system in augmented here and synergized by ubiquoinol, Jarrow, QH absorb 100 mg, Los Angeles, Calif., the capsule is broken and added to the transdermal cream as well as the oral preparation. addressing cancer and unprocessed debris such as in neurologic diseases, as with Parkinson's disease, Alzheimer's disease

### BRIEF DESCRIPTION OF THE DRAWINGS

FIG. 1 is a photograph showing the poor condition of an equine veterinarian area called the fetlock, an area of poor micronutrient circulation where the calf ends and the ankle begins, before subject composition immunotherapeutic agent (IA) F.A.S.T.® treatment.

FIG. 2 is a photograph showing the “incredible healing” of the same equine fetlock that took place after administering F.A.S.T.® locally.

FIG. 3 is a photograph showing the damaged tissue of the area below the calf of a human leg with poor micronutrient circulation, for the first 4 weeks without F.A.S.T.® treatment, requiring a full thickness skin graft.

FIG. 4 is a photograph showing the healed tissue of the area below the calf of the same human leg 4 weeks after using F.A.S.T.® locally and systemically, averting a full thickness skin graft.

FIG. 5 is a radiograph of pre-treatment intense ileitis with Indillum-111 radioactively tagged inflammatory cells. Pretreatment abdominal scintigraph of a patient with active IBD Crohn's Disease, showing intense inflammatory activity in the right iliac fossa representing ileocecal inflammation. This radiograph demonstrates the T-cells as cytotoxic and chemo-attractant effects of disease tissue associated with severe inflammation of ileitis of Crohn's disease (70% of Lymphocytes in the circulating peripheral blood stream are T-Lymphocytes, Indillum-111 radioactively tagged and represented here radiographically as the scintillating inflammatory cells attachment, the chemical engineering bases of IBD Crohn's inflammation).

FIG. 6 is a post-treatment repeat radiograph of Indillum-111 radioactively tagged leukocyte ileitis cleared after 4 weeks. Repeat radiograph in same patient after 4 weeks of treatment with elemental diet as a food substitute, Verma's work using ½ food and ½ elemental feed showed 50% efficacy, since elemental feed orally depends on it being a food substitute. This radiograph demonstrates the therapeutic effect in providing the reversal and disease resolution by immunotherapeutics of the T-cells and their cytotoxic inflammatory affinity and the chemo-attractant effects of diseased tissue.

FIG. 7: averting the need for a liver transplant from congenital viral inflammation and associated inflammatory biliary atresia in an infant, with progress improvement and subject composition immunotherapeutic activating the patient's own stem cells noninvasively and without the use of foreign cells, shown in four stages: top left, a newborn with serious jaundice that needed liver transplant—the infant appears to be black due to intensity of jaundice; top right, two weeks into treatment; bottom left, after 3 months of treatment the infant no longer needed a liver transplant; bottom right, a healthy child with a positive future.

FIG. 8: left to right, periodic table examples of 16 essential nutrients and micronutrients from textbook McGraw; Girsh found and listed in this patent 24 essential micronutrients and trace nutrients of human tissue.

FIG. 9 shows plant examples of elemental deficiencies: top picture, smaller left plant is nitrogen deficient, larger right plant is nitrogen sufficient; middle picture, smaller left plant is phosphorous deficient, larger right plant is phosphorous sufficient; bottom picture, smaller left plant is calcium deficient, larger right plant is calcium sufficient.

FIG. 10 shows the cell cycle and bioenvironment micronutrient providing cell tissue assembly system 1, 2, and 3.

FIG. 11 shows bioenvironmental micronutrient of the 24 elements highlighted of the periodic table of 92 elements.

FIG. 12 shows a breast cancer cell in the spinal fluid en route to metastasizing to the brain and the biochemical micelle conformity of lipophilic low HLB (hydrophilic/lipophilic balance) surfactant. Lipophilic surfactants with lumps of emulsifier with surplus of water have never been shown to kill cancer rather increase cancer cell growth, 50% in cases of HLB of 2 PGPR (polyglycerol polyricinoleate). These surfactants are characteristic of cancer as therapeutic reversal of 70-100% kill studies in following tables of 24 to 48 hours incubated with the therapeutic hydrophilic surfactant of bioenvironmental micronutrients of subject composition preferred treatment of cancer using this algorithm. 40% of studies had an anticancer effect known undesirable effects, whereas use random computer search of chemicals 1 in 3 million anticancer chemicals are found (10,000 fold less than subject composition), without the features of freedom of possibly another 10,000 undesirable effects. Offered here, unique to the prior art, is the first documentation of a biochemical trend: “The search for a common qualitative biochemical defect to explain neoplasia has to date been unsuccessful.” (Rubin & Fuber, Pathology, copyright 1988 JB) FIG. 13 shows liquid crystal micelle with a high hydrophilic/lipophilic balance (HLB) of 8-10 to >13 that kill 70-100% of patient's cancer cells of as Hela cells and prostatic cancer cells, above the horizontal line. Below the line shows low HLB characteristic of cancer and in 4 experiments in tables show to increase cancer cells with polyglycerol polyricinoleate (PGPR) surfactant with HLB of 2 by 50% and after experiments with low HLB<6 by about 25% increase in cancer cells.

FIG. 14 is a summary of lipophilic chemicals without anticancer hydrophilic surfactant therapeutics of subject composition immunotherapeutics.

FIG. 15 shows the magnet-like relative HLB forces in formation of final biologic configuration of protein molecules with table and counter clockwise presentation of progressive use in templating protein as string of pearls then magnet-like forces of amino acids finally of the final structure in 3D of protein with the lipophilic amino acids in the centre and the hydrophilic amino acids on the outside.

FIG. 16 shows high HLB blends of amino acids specific by the genetic code of human and mammalian tissue, 2 extra cellular matrix (ECM), and phospholipids wherein each achieved an average of about 81% cancer cell kill rate of cancer cells from the patient's prostrate, and in the form of transdermal cream with 24-48 incubation kill of 80% of prostatic cancer, all performed in one South Carolina laboratory with the exception of the Florida laboratory of transdermal cream study of prostatic cancer and same results. This shows the uniformity and reproducibility of this work with 2 different surfactants (one from egg yolk and one from plant soy source) but identical biochemical blends. The first amino acid group in the table was not used, only the following three blends were used for requirements of cell tissue assembly system (See FIG. 10).

FIG. 17 shows the biophysics of mammalian and human tissue hydrophilic surfactant surface-to-surface interaction applied to cancer treatment, microscopic subspecialty viewed as a micro-cosmonaut from a biophysics and biochemistry stand point. The top left table shows Grape Seed extract (Now Foods, Bloomingdale, Ill.) 1 capsule 2-3 times daily; use orally for cancer application. Top right table shows the three subject composition components averaging 81% cancer cell kill in 24-48 hours of 2 types of cancer in 2 different laboratories, South Carolina and Florida. It is believed to kill many types of cancer in vivo and in vitro. This immunotherapeutic agent (IA) is now available for many types and many locations of cancer. The bottom table shows 1-80 diluting Liposyn with 1.2% egg yolk phospholipid greatly increases its HLB and hydrogen bonding forces of 4 bonds to 1 water molecule seen in 3D as a hexagon shaped H₂O configuration. This clinical effect of high HLB can also be tracked with measurements of hydrophilicity and hydrogen bonding force by measuring electrical conduction in barefoot patient at the same time as measurements of his weight. The conductivity of fat is measured by impedance, as the nonconductive effect of lipophilicity, and with the treatment of the multiple myeloma patient is went from 25.6% to 24.4% (25% is marker for early signs of obesity, the body mass of fat versus water). The water increased from 55.4% electrical conductivity in contrast of 54.6% as in index of its hydrophilicity and hydrogen bonding, using the Thinner™ scale manufactured by Con Air, East Windsor, N.J.

FIG. 18 shows biophysical and biochemical in vitro demonstration basics for cancer cell kill, which due to activation of surface-to-surface enzymatic activation of apoptosis is a 73-100% kill of cancer cells not affecting the adjacent normal cells. Our further stem cell molecular embryology studies reveal also believed as method of activation by fertilization of templating monomer amino acids to polymer proteins by hydrophilic surfactant by phospholipase in the head of sperm.

FIG. 19 documents subject composition hypothesis of lowest HLB causes adverse effect in cancer cell very low HLB PGPR which supplies lowest HLB of 2 with greatest increase (59%) of cancer cells. Addition of very dilute high HLB of 15 hydrophilic and associated hydrogen bonding neutralized low lipophilic HLB of PGPR. Significant affect of high HLB, e.g. amino acids lysine, among the highest HLB hydrophilicity amino acid.

FIG. 20 shows cancer cell lethality using non lethal substance; as expected, the lipophilic surfactants did not kill the cancer cells and the hydrophilic surfactants significantly killed the cancer cells.

FIG. 21 shows a treatment example of subject composition for oral use.

FIG. 22 shows the dental exam of gingival dental margins (sulcus). If dental probe goes from 2 mm. 2 mm sulcus gum margin is normal, 6 mm is abnormal (periodontal disease).

FIG. 23 June 2001 letter: This subject composition has been uniquely received by the Regulatory Affairs agency as “exciting and novel” and is further unique in that it is extensively reviewed by the Regulatory Affairs agency, and because of our spreadsheets reported scientific studies documenting indications; this micronutrient medicament becomes a drug in vivo.

FIG. 24 January 2011 letter: By presenting the chemical moiety and its therapeutic rationale as activation of cell tissue assembly system and the timeline in patient care further provides documentation where this becomes a steroid substitute and steroid sparing agent.

FIG. 25 shows an example of splinter hemorrhaging as an indication of low grade septicemia.

FIG. 26 shows a second example of splinter hemorrhaging as an indication of low grade septicemia.

### DESCRIPTION OF PREFERRED EMBODIMENTS

The compositions of the subject invention (available from Immunopath Profile Inc. (IPI)) provide an Immunotherapeutics Agent (IA) tissue integrity, innate immunity, and associated adaptive immunity. The pharmacologic basis for this immunotherapeutics in bringing about disease resolution.

IA not only brings about disease resolution, but also permits the co-use of pharmaceutical medications and minimizes, in the same fashion, the risks of side effects.

Therefore, we can now look at the tachyphylaxis effect (a rapid decrease in the response to a drug due to previous (long term) exposure to that drug) “honeymoon period” that was first discovered for insulin as a similar mechanism in other key drugs such as corticosteroids. We have shown here, in the case of steroids and management of Crohn's disease, that IA becomes a steroid sparing/substitute therapeutic documented by a wound where the tissue appeared to fall apart and the side effect of steroids interfering with wound healing became prominent.

We see the same effect in the remarkable drug, Levodopa, in the treatment of Parkinsonism with the side effects of stroke, heart attack, and substantia nigra, basal ganglia damage. I have recently seen this in the treatment of atrial fibrillation with Cardizem (average oral daily dose is 180 milligrams (mg) to 360 mg, divided into three or four smaller doses, and 0.25 mg/kg actual body weight as a bolus administered over 2 minutes, [125 mg (25 ml)/100 ml] [Titrate] [Total volume=125 ml] intravenous) (side effects page 425 PDR 64^{th} edition, 7 of the patients died) The patient post-normalized on the first dose, and on the second dose the patient had pain, inability to walk, and weakness. The total studies were almost 30,000 in number and BIOMARKERS TISSUE DEFICITS AND THERAPEUTIC TARGETS FOR CARDIAC ARRHYTHMIA Cardiac arrhythmia nutritional deficiencies, 496 studies compared with:

The chemical moiety component, of the compositions of the subject invention, to detect biomarker tissue defects in the composition's immunotherapeutic agent (IA) directed to the treatment of these biomarker tissue defects in reestablishing tissue integrity, innate immunity, and associated adaptive immunity and disease resolution 1,137 studies of efficacy with acknowledgement of tissue deficits.

29,604 studies of efficacy of the compostion(s) chemical moiety components (without active acknowledgement of tissue deficits) in the composition(s) immunotherapeutic agent (IA) directed to the treatment of these biomarker tissue defects in reestablishing tissue integrity, innate immunity, and associated adaptive immunity and disease resolution.

The use of the transdermal cream on her legs and the oral subject composition several times daily, and the use of the B vitamins such as B1 100 mg, Omega-3 fish oil 1000 mg, magnesium in the form of magnesium taurate, brought about dramatic disease resolution. Vitamin B₁ was cited as the most critical of preventing and reversing arrhythmia in starvation studies. Omega-3 fish oil was shown pre-clinically to be effective in the animal heart studies: after it was digested from and enzymatic release from glycerol was not only shown by Leaf in vitro in the isolated beating animal heart muscle to prevent toxicity effects of toxic chemicals. On our chemical moiety orientated spreadsheet where we listed more than 1,400 studies documenting the efficacy of magnesium.

Based on this dramatic experience of disease reversal and resolution of undesirable side-effects form drugs such as Cardizem and our extensive research indicating that diseased or damaged tissue has various modalities of origin, such as but not limited to, a side-effect of a drug, so vital such as Cardizem in correcting a life-threatening arrhythmia that the co-use of subject composition including the transdermal administration of the most advanced Resveratrol transdermal cream illustrated in Example Group 3, and the oral medication listed in Example Group 1. With complete components listed in the spreadsheet of indicated diseases, giving a more complete list, provides by maximizing and optimizing efficacy and safety in the foregoing subject composition free of undesirable effects not only for the treatment of disease but for averting these undesirable side-effects. So doing in this example the patient was not only free of her disease, but also free of the side-effects that she no longer had to use the walker she went home with and was free of pain in her legs, and was able to avoid using the Cardizem as an outpatient because of the normalization of her vital signs and pulse based on these measures. The measures includes a daily monitoring of the vital signs that have been found to be normal, and when the blood pressure is increased the lower risk measures of continued subject composition and Benicar when the blood pressure was above abnormal range of 140 or more. Since her pre-hospitalization condition began with uncontrollable refractory control in failure to respond to one of three doses of 0.4 mg nitroglycerin, Nitrostat, in this case by reducing the blood pressure level we were able to stop the angial discomfort and pain on the coronary arteries.

A similar tachyphylaxis effect also described as a “honeymoon” effect with post-marketing side effects that include: pain, inability to walk, and in the case of predisposition to Parkinsonism, damage to the basal ganglia.

The subject composition(s) chemical moiety component efficacy bringing about disease resolution by correcting of deficient tissue biomarkers thereby reconstituting tissue integrity (a most important mechanism of providing disease resolution), innate immunity and adaptive immunity

In conclusion, the co-use of this subject composition immunotherapeutic agent (IA) medication not only reverses diseased or damaged as a broad-based regenerative medicine but also prevents and addresses undesirable side-effects with vital medications that may be required in the life time management of a patient. This co-use therapeutic protective effect in therapeutic care would also extend the patent life of these vital pharmaceutical products.

The first discovered this concept of tissue resistance and its structural and functional identification occurred during the inventor's first year of practice as an associate professor at Temple University. In my specialty in immunology and allergy I recognized that allergy was an ‘overactive immune mother’ acting as innate immunity in rejecting pollen, mold and food allergies such as milk, chocolate, and eggs. I felt that although the cancer cell may occur, this ‘overactive immune mother’ was also rejecting it. I found there were 15 articles presented to document this concept (ref 1958)

In 2010 the Japanese found the tissue integrity and its associated innate immunity and document that biochemically by extending on the same observation that I have made in that the associated innate immunity can be indentified by certain biochemicals. They showed the TH2 phenomenon and TH1 in sarcoid patients. They were able to show this difference and what makes this disease so mild since allergic disease is endowed with reversibility and is also explained by this adaptive immunity. In the case of the sarcoid, it doesn't coexist with allergy, the innate immunity is very disrupted and the response triggers the development of TH1 immune responses and reduced expression of regulatory T cells also contribute to the augmented TH1 disrupted innate immune response. Conversely, allergic disease, or atopy, is characterized by the production of a specific immunoglobulin E response towards environmental or food allergens in association with combating and minimizing diseases such as cancer, which as we have said from my observations that has been continued since 1958, that there is a significantly lesser incidence of cancer in the allergic population. During the first year of practice in allergy and asthma the inventor only encountered one patient who had cancer, whereas in internal medicine it was encountered in one patient a week. The immune response is characterized by the production of TH2 (cytokines such as IL-4 and IL-13) in addition to the immunoglobulin E response. This is in contrast to the corrosive, but apparent response protection of sarcoid making it a more serious disease and TH1 cytokines, as components of the innate immunity include gamma, IL-12, IL-18, and tumor necrosis factor alpha that appear to be essential for the granuloma formation. Recent insights into the immunopathogensis of sarcoidosis suggest that a disrupted innate immune response triggers the development of TH1 immune responses. Reduced expression of regulatory T cells also contributes to the augmented TH1 immune response with the cytokines so mentioned. This reduction in the expression of disease is also atopy in association with rheumatoid arthritis and multiple sclerosis in association with allergic disease such as house dust mites specific IgE response. This shows a specific biochemical pathogenesis trend.

The diseases amenable to treatment include: wound healing, healing of burns (inclusive of oncologic radiation burns), and decubitus ulcers, all requiring the same challenge of wound healing. Second is stem cell healing activated by the immunotherapeutic agent compositions of the subject invention and by following not only reestablishing tissue integrity but also innate immunity and its associated adaptive immunity, but also following molecular embryology in regard to the molecular structures that underlie key cell biology systems. Finally, we have diseases that include cancer (a wound that does not heal then goes on to metastasis) as an extension of wound healing.

First Use of IPI's Immunotherapeutic Agent (IA) Transdermal Cream

Treatment to advance the healing of wounds, major burns inclusive of radiologic burns in oncologic care, and healing of decubitus ulcers.

### Rotator Cuff Tear:

2 male patients, age 56, and age 80, had surgical care averted with the use of subject composition therapeutic lotion applied topically to the affected area. Diagnosis was confirmed clinically by examinations which also included an MRI examination in one patient.

Symptoms of pain and weakness in abduction of the left arm and forearm (Movement by the patient, abduction, of upper extremity 90 degree angle away from the body). Diagnoses of both patients' were confirmed by orthopedic examination. The first patient's diagnosis was also confirmed by nuclear magnetic resonance examination. Patients progressively improved over a period of three to four weeks. Symptomatic improvement was noted in regard to pain reduction, within five to ten minutes after local application of the medication without the need for analgesic, as clinical evidence of normalization of neuronal flow.

A composition of the subject invention was able to provide the treatment and therapeutic disease resolution with their transdermal cream where other medications were unsuccessful.

The preclinical animal studies of the subject immunotherapeutics were supervised by one of the leading equine veterinarians in the United States providing humanitarian ethical methodology for documenting the Immunotherapeutic Agent (IA) known as F.A.S.T.® anti-inflammatory disease resolution:

This “transdermal stem cell repair cream supplied has been very successful in over 27 cases of tendosynovitis and joint capsular synovitis of the Equine Species. Many of these cases were refractory to other therapies including steroids, non-steroidals, shock waves, IRAP (inflammatory receptor antagonist protein), and hyaluronic acid. It appears that this product helps these cases get over the final phase of tissue repair in tissue damaged so severely that the body cannot supply raw materials necessary for healing, and consequently reduce inflammation necessary for tissue homeostasis”.

This eminent equine veterinarian was invited to present his findings of therapeutic response to IA to the monthly Orthopedic Society meeting focused on advancing sports medicine therapeutics. Based on his presentation the orthopedic surgeons requested a continued update for a venue of IA therapeutics market availability.

The patent citation designed to further stimulate healing disease resolution and the patient's own stem-cell activation non-invasively regarding IA further synergized by the plant-derived adaptogen phytoalexin grape skin derived Resveratrol (muscadine grape, organically grown anti-oxidant full spectrum polyphenol flavonoids, proanthocyanidins (OPC's), anthocyanins) not only expedited healing in that the healing time response now ranged from 15 minutes to several hours (in contrast to 1-2 weeks) and “was incredible on a skin transplant that we performed on a horse. You can't even tell that he has new skin.”

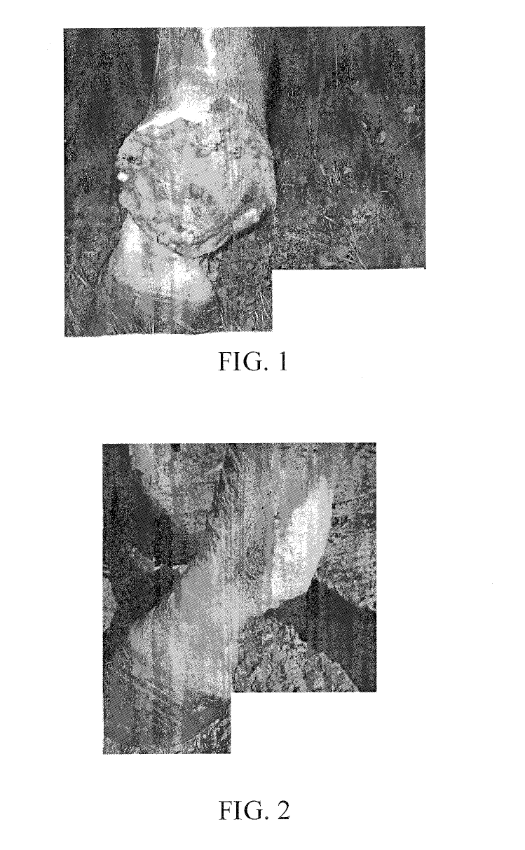

This recent equine veterinarian follow-up provided the following quotes concerning therapeutic disease resolution with IPI's transdermal cream. (See FIGS. 1 and 2).

This area of poor circulation is referred to the fetlock joint in the equine species. An analog in humans would be the area below the lower calf where there is the same complication of poor circulation (see images below).

These lesions of the horse and human are presented here for comparative purposes of not only a similar injury with similar complications, but the same incredible healing and disease resolution. These micronutrients that are normally present and carried in circulation in enhancing recovery and disease resolution based on the compositions of the subject invention and other filed patents, have been recognized by the Regulatory Affairs Agency as a medicament that becomes a drug in-vivo. Therefore this explains the efficacy of presenting it in-vivo directly to the diseased area.

This focus on vascular delivery of micronutrients to the tissue offers a therapeutic opportunity to peripheral vascular disease such as leg ulcers.

We have further found that IA can be used while waiting for an organ or skin transplant. This case illustrates this degree of disease resolution in not only pre-operative, operative, and post-operative care but also applicable in averting the need for an organ or skin transplant, in the following case orally administered.

Non-surgical Wound Healing and Tissue Regeneration: Patient Scheduled for Skin Graft, Averted with IPI Therapeutics.

Applied to damaged tissue, exemplified in major wound healing, averting a full thickness skin graft.

Since very little debridement was required it is evident that this subject composition immunotherapeutic had a similar apoptotic effect as occurs in normal tissue

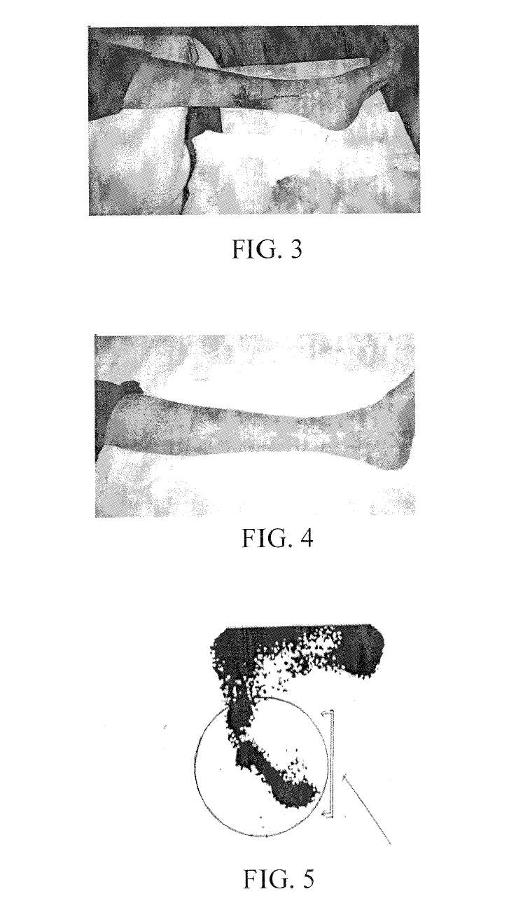

“A picture is worth 1,000 words” to be further described with the activation of apoptosis. (See FIGS. 3 and 4).

Teahon's 1991 article (discussed in the designation request) deserves special mention as it objectively highlights the therapeutic effects of elemental diet. This study examined whether treatment of acute Crohn's disease with an elemental diet improves intestinal integrity and inflammation. Teahon proposed a mechanism by which the elemental diet heals tissue, centering around the importance of the integrity of the intestinal barrier function, and reduction in inflammation. We provide below our comments to the radiographs from this article.

Upon reflecting on this case, the healing effect of F.A.S.T® on the patient's leg wound in October 2000 probably mimics the beneficial effects observed for her Crohn's disease. Her leg was so severely wounded as to cause her plastic surgeon to recommend a full thickness skin graft. Her prognosis was not optimal since the corticosteroid she was taking for Crohn's disease was predicted to have impaired healing both of the leg wound, and the site of skin donation. Instead of the skin graft, a composition of the subject invention was administered both transdermally and orally and her dosage of steroids was reduced. Her leg wound healed in 4-6 weeks with minimal debridement and no sutures (steri-strip wound apposition was successfully used to close the wound). It remained healed and is free of a scar despite the magnitude of the wound (photo evidence).

FIG. 5. Pre-treatment: Intense ileitis radiograph of tagged inflammatory cells. Pretreatment abdominal scintigraph of a patient with active IBD Crohn's Disease, showing intense activity in the right iliac fossa representing ileocecal inflammation. This radiograph demonstrates the T-cells as cytotoxic and chemo-attractant effects of disease tissue associated with severe inflammation of ileitis of Crohn's disease (70% of Lymphocytes in the circulating peripheral blood stream are T-Lymphocytes, represented here radiographically as the scintillating inflammatory cells attachment, the chemical engineering bases of IBD Crohn's inflammation).

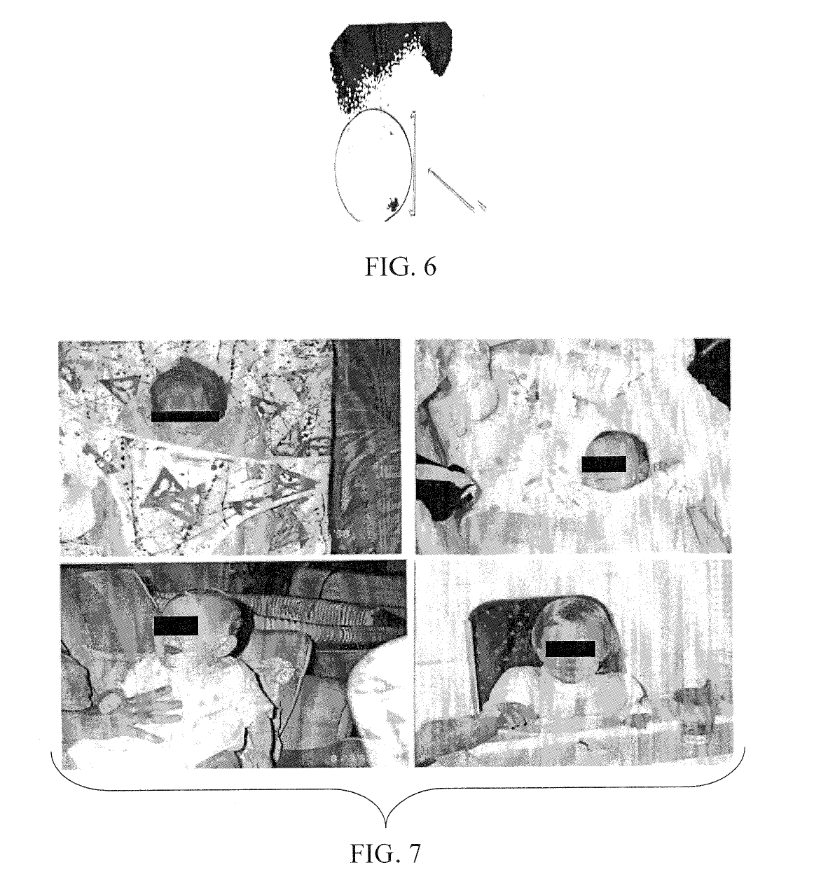

FIG. 6. Post-treatment: Repeat radiograph of ileitis cleared after 4 weeks. Repeat radiograph in same patient after 4 weeks of treatment with elemental diet. This radiograph demonstrates the therapeutic effect in providing the reversal and disease resolution by immunotherapeutics of the T-cells and their cytotoxic inflammatory affinity and the chemo-attractant effects of diseased tissue.

However the compositions of the subject invention can also contain additional components. To our knowledge, nobody has included in a single treatment plan all of the components described in the orphan drug designation request for compositions of the subject invention. Our hypothesis is that the elemental diets discussed in the published literature and described in the designation request were “only” as effective as steroids because the elemental diets were not provided in the optimal molar ratio, and did not include important additional components that are necessary for cellular function and regeneration. In particular, elemental diets lack the components of the cell membrane and extracellular matrix that are provided in the compositions of the subject invention. By supplying the body with the chemical components of the extracellular matrix, such as cartilage containing chondroitin sulfate and collagen, tissue repair and anti-inflammatory activity is enhanced since the extracellular matrix provides tissues with structural support and directs cell migration, attachment, and organization. Cell membrane formation and repair is enhanced by the simultaneous administration of phospholipids, essential fatty acids, and EPA. In sum, the compositions of the subject invention can be designed to repair diseased human tissue and enhance the formation of healthy tissue. Since all of the components are necessary for cellular function, all of the moieties in compositions are thought to be essential and “active.” We discussed these other moieties in the designation request, and provided references for many of the components that are not already part of the elemental diets described above. Additional references can be provided on request.

It is noted that there are several examples of products that are designated as orphan drugs containing more than one “active moiety.” These include “branched chain amino acids” for the treatment of amyotrophic lateral sclerosis, Elliotts B Solution (calcium chloride; dextrose; magnesium sulfate; potassium chloride; sodium bicarbonate; sodium chloride; sodium phosphate, dibasic, heptahydrate) for use as a diluent in the intrathecal administration of methotrexate and cytarabine for the prevention or treatment meningeal leukemia and lymphocytic lymphoma; Extraneal (icodextrin 7.5% with “electrolytes”); M.V.I.-12 (ascorbic acid; biotin; cyanocobalamin; dexpanthenol; ergocalciferol; folic acid; niacinamide; pyridoxine hydrochloride; riboflavin phosphate sodium; thiamine hydrochloride; vitamin A; vitamin E) for the prevention of vitamin deficiency and thromboembolic complications in people receiving home parenteral nutrition and warfarin-type anticoagulant therapy; and Nutrineal (peritoneal dialysis solution with 1.1% “amino acids”) for use as a nutritional supplement for the treatment of malnourishment in patients undergoing continuous ambulatory peritoneal dialysis. Presumably, the Office of Orphan Product Development (OOPD) agreed that all of the components in these other products were “active moieties.”

When I was requested to see this patient in 2001, she had been on continuous corticosteroid treatment for Crohn's disease for thirty years. The required dosage of corticosteroid was titrated to a minimal dose in accordance with her rate of recovery from severe flare-ups, which would occur every one to three months. At that time for the flare-ups the dosage was 4-6 tablets per day for one to three days. Then in a count down fashion every two days the dosage was reduced by one tablet every 1-2 days to finally achieve the dosage of one to two tablets daily. Thus she was taking 1-6 tablets daily, and the larger dosage was dependent upon corticosteroid requirements to reverse severe flare-ups.

After the patient injured her leg, she requested a treatment that would help avert the need for a full thickness skin graft (since half the sutures were dehiscing) as well as replace the steroids that were interfering with the healing process and her recovery. Transdermal and systemic oral administration of compositions of the subject invention were used in this case and her steroid dosage was simultaneously decreased (not increased). It was found clinically that the compositions not only averted the need for full thickness skin graft and the risk of creating another wound (to obtain skin for the graft, which also would have had a reduced ability to heal), but also was found to serve as a steroid substitute. About four weeks after the treatment was initiated, and at the time of the second photograph in the designation request, she had decreased her steroid consumption by about 25% (1 tablet and ½ tablet on alternating days) and now requires only 1½ tablets weekly (½ tablet 3 times weekly).

In retrospect we can now further see another therapeutic action of these compositions: they can progressively replace the tissue and tissue fluid loss of 3-6 or more watery diarrheal bowel movements per day with major flare-ups and at times in between major flare-ups. Therefore, have helped not only to replace the tissue deficiencies of the wound but also for the tissue break down associated with IBD Crohn's disease.

We note that OOPD defines a pediatric population as being 16 years of age or younger.

Before the initiation of the compositions of the subject invention, the patient was taking Aristocort 4 mg, one to two tablets daily, with higher doses necessary to treat flare-ups of Crohn's disease. She was also taking Azulfadine at a low dose.

In October 2000, the patient sustained a severe wound, out of proportion to the trauma, to her calf, as previously documented. Her physicians believed that her constant use of steroids would interfere with wound healing and wound dehiscence, and that is when I began administering the compositions of the subject invention both orally and transdermally. On the day before treatment was initiated in 2000, she was taking her baseline of Aristocort (one or two 4 mg tablet/day). On the day that GIR114P was initiated, the patient was then given a more than 25% reduction of corticosteroids: one tablet the first day and then a half of a tablet the second day, and continued in that alternate fashion. That dose of corticosteroids was gradually decreased over the next several years as therapy with the compositions continued. For several years, she was taking one Aristocort tablet ever other day, which was later reduced again to one-half tablet every three days. Within the last few months, she has stopped taking corticosteroids altogether. She has continued taking a low dose of Azulfadine and compositions of the subject invention.

Unfortunately, her primary physician has retired from practice and despite a diligent effort, I have been unable to obtain copies of his notes and records, or those of her gastroenterologist. Therefore, I am unable to provide any additional detail regarding the exact dates these reductions in Aristocort occurred. What is remarkable, however, is that despite these reductions in corticosteroid use, she has not had a single flare-up since initiating treatment with the compositions of the subject invention in October 2000.

Before the initiation of therapy with an embodiment of the subject composition in October 2000, despite the fact that she was taking low dose of Azulfidine and Aristocort 4 mg, 1-2 tablets per day, she had flare-ups every one to three months. Flare-ups were treated by increasing her dose of corticosteroids for about four to six days. After resolution, the steroids were reduced back to about one to two tablets per day.

As mentioned above, treatment was initiated in October 2000 and she was weaned to the most minimal dose of steroids over the next several years. Since the initiation of therapy in October 2000, she has not had a single flare-up. Therefore, there has been no referral to a gastroenterologist.

Apr. 29, 1983: Diagnosis of Crohn's disease was well established and confirmed at the Cleveland Clinic by operative and pathologic findings as well as radiological findings prior to surgery.

Oct. 28, 2000: As I have previously described, her leg was severely wounded. Although not recorded in the plastic surgeon's notes, this is when treatment with compositions of the subject invention, both orally and transdermally, was initiated and at the same time Aristocort was progressively, but very gradually reduced.

Dec. 5, 2001: No mention of Crohn's disease.

Nov. 22, 2005: Physician's note indicates that Crohn's disease is under control, and patient takes Azulfidine.

2007-2010: Laboratory reports showing inactivity of Crohn's disease (normal sedimentation rate, normal C-reactive protein, normal CBC, and normal chronical index of Crohn's).

Aug. 31, 2010: She reports no symptoms of Crohn's disease and is not taking any medications for the treatment of Crohn's disease.

In conclusion, prior to October 2000, despite being treated with corticosteroids, this patient had several flare-ups every year. Flare-ups were treated by temporarily increasing the dose of steroids. Therapy with compositions of the subject invention was begun in October 2000, and corticosteroids were gradually reduced over the next several years to the most minimal dose. This patient has not taken steroids at all for the last few months. The patient has not had a single flare-up in the last ten years.

Upon reflecting on this case, the healing effect of the subject composition on the patient's leg wound in October 2000 probably mimics the beneficial effects observed for her Crohn's disease. Her leg wound was so severe as to cause her plastic surgeon to recommend a full thickness skin graft. Even then, her prognosis was not optimal since the corticosteroid she was taking for Crohn's disease were predicted to have impaired healing both of the leg wound, and the site of skin donation. Instead of the skin graft, the subject composition was administered both transdermally and orally and her dosage of steroids was reduced. Her leg wound healed in 4-6 weeks with minimal debridement and no sutures (steri-strip wound apposition was successfully used to close the wound). It remained healed and is free of a scar despite the magnitude of the wound (photo evidence on page 11 of original application of Office of Orphan Products Development). Likewise, her gastrointestinal tract probably healed in a similar fashion. The Teahon article provided earlier provides strong support for this conclusion. Just as steroids would have interfered with her leg injury, they likely interfered with the healing of the wounds in her intestinal tract. When considering the case of pediatric Crohn's disease, the reduction (or elimination) of steroids is particularly important because of their pronounced and well-documented effects on growth and pubertal development.

Moreover, the subject compositions provided this patient with all the elements necessary to heal the wounds and regenerate the tissue in both anatomic locations. Although objective evidence of remission can not be provided for this patient, Dr. Teahon objectively showed that an elemental diet reduces intestinal permeability and inflammation. She concluded that the mechanism by which an elemental diet is effective in patients with Crohn's disease is centered around “the importance of the integrity of the intestinal barrier function.” Importantly, as mentioned throughout this letter, this patient has not had a single flare-up of Crohn's disease in the last 10 years, despite a gradual reduction and eventual elimination of all corticosteroids. I am unaware of any case showing such a profound and sustained response of treatment without known and undesirable effects.

(See Teahon K, Smethurst P, Pearson M, Levi A J and Bjarnason I. The effect of elemental diet on intestinal permeability and inflammation in Crohn's disease. Gastroenterol. 1991; 101: 84-8.)

The intensity of delivery of these circulatory micronutrients for disease resolution can be illustrated by another patient's experience where he recovered from veritable spinal cord trauma after falling out of a tree. He was incapacitated from walking until he obtained treatment wherein his blood pressure was increased through exercise to increase the delivery of nutrients in his body to the damaged tissues of his spine.

A similar injury (but without spinal cord trauma) can be seen in a male patient, 67 year old, thoracic surgeon who fell off an 18 foot ladder in 2003 and sustained a fracture that destroyed his L1 vertebra, (often referred to as a “blow out” fracture because of the complete destruction of the body of vertebra. Since 2003, he has had pain every time he rotates the axial alignment of his vertebral column (best exemplified as getting in and out of a car). He administered an IA composition of the subject invention as a transdermal cream locally (an amount the size of a quarter) 2-3 times daily for 2 weeks. The patient has recovered sufficiently that he was the first timeable to go through axial rotation without any pain. This represents the efficacy of pain management in normalizing the neuronal flow, without other medication.

Fixed numbers show that this chemical moiety has a high probability of efficacy because he was pain free 28 of 30 days and probability factor is significantly greater than 0.0001 which is a high probability in contrast to having pain symptoms from any axial movement in 28 out of 30 days. This is the obverse of being symptom free 28 out of 30 days.

A male dentist, mid 60s, was diagnosed with degenerative osteoarthritis of the left knee in 2001, by an orthopedic surgeon at the Henry Ford Health System in Farmington Hillsi Mich. At that time, he was 49 years old, and was working 55 hours per week at his dental practice. The orthopedic surgeon advised a complete knee replacement since the joint space was minimal as per the M.Rt. The meniscus had worn away from athletics and job requirements. The option was chosen of using occasional steroid injections in times of severe inflammation along with the daily use of Celebrex until he could no longer function, rather than have such an irreversible traumatic operation, at his age. The doctor agreed. His quality of life was affected as was his comfort during work.

A few years later, the right knee ligaments were starting to weaken resulting in much pain and dysfunction, the now 57 year old patient's gait was hobbled to a severe limp. Ibuprofen of 600 mg was helpful but not therapeutic.

A composition of the subject invention in lotion form was applied twice daily after warming the area with a moist-heat heating pad for a few minutes. Within days, there was noticeable relief in both swelling and pain.—My mobility was already improving.

Plans for a knee replacement were put on hold. The patient subsequently suffered a ligament pulled ligament in the groin. The ligament was also treated with the same composition In three days the pain was gone. Averting the Need for a Liver Transplant:

The synthetic biopharmaceutical immuno-therapeutic agent (IA) of the subject invention is also particularly effective for treating congenital biliary atresia (congenital viral hepatitis). Congenital biliary atresia (congenital viral hepatitis) is a stem cell focused disease.

A small population of stem cells located in the junction between liver cells and the smallest segments of the biliary tree may differentiate into liver cells and biliary duct epithelium and are known to participate in regeneration that occurs in certain forms of hepatitis. Thus, in congenital biliary atresia (congenital viral hepatitis) the stem cells are able to grow new tissue that are located in close proximity to the damaged tissue.

The damaged tissue has periductular inflammation and fibrosis. The locus and focus of maximal disease is at the junction of the smallest ductules of the biliary tree and the adjacent liver cells (hepatocytes), which is also the location of stem cells that are capable of regenerating liver tissue cells.

This was first applied to a child that was scheduled for a liver transplant. The child had congenital biliary atresia, but we can now look at it with more advanced technology as congenital viral hepatitis. We were able to reverse this disease by using this synthetic biopharmaceutical system, thereby averting the need for a liver transplant.

The biliary tree may be looked upon as an anatomic continuum of the small bowel. In view of this anatomic continuum the similarity of small bowel healing and biliary tract healing continuum is presented here. (See FIG. 7 showing 4 pictures: top left, a newborn with serious jaundice, needed liver transplant. Child appears to be black due to intensity of jaundice; top right: two weeks into treatment; bottom left: 3 months of treatment no longer needed liver transplant; bottom right: healthy child, positive future).

ACHIEVING INNATE IMMUNITY THAT COUNTERS THE DISRUPTED INNATE IMMUNITY OF DISEASE THROUGH ESTABLISHING NORMALIZATION OF TISSUE INTEGRITY BY THE SHORT CUT OF UTILIZATION OF INNATE IMMUNITY OF GRAS ITEMS SUCH AS:

Biomarker Tissue Deficits and Therapeutic Targets for Disease

Nuclear genetics represent the architectural plan of the cell tissue assembly system and the apoptotic waste disposal system of worn, diseased, damaged, and abnormal tissue, inclusive of all degrees of inflammation damaged tissue such as but not limited too inflammatory bowel disease (IBD) and cancerous tissue. The drawing board is all the basic science of medicine, now inclusive of molecular embryology and particularly guided by bio-chemistry, pathology, pharmacology, and pharmacognosy and so represents the blueprint and drawing board for problem-solving and disease resolution.

This will be directed to the successful management we have achieved of wound healing, IBD, and cancer (all products of progressive severe inflammatory damage, inclusive of pain management). By directing and producing components of tissues to reestablish cell tissue apoptotic waste disposal systems, a defensive system of disease resolution, in contrast to using a primarily offensive and tissue damaging system.

This is inclusive of conceptual unique interdependent interrelationships of composition to expedite the achievement of disease resolution, analogous to winning a championship chess game (not yet in the public domain) which includes unique component arrangement of the winner and taking advantage of the component deficiencies of the opponent, in this case, disease.

The analogy here is strictly devoted to uniquely directing molecular components to the cell biology systems required for disease resolution, in the spectrum of diseases from the major organ disease such as but not limited to liver and kidney, neurologic system, diseases of poor or guarded prognosis, the most severe disease of this inflammatory spectrum of diseases that almost heal and doesn't heal.

### Biomarkers Tissue Deficits and Therapeutic Targets for Multiple Myeloma

Multiple Myeloma nutritional deficiency studies, 138 compared with:

The chemical moiety component of the compositions of the subject invention to detect biomarker tissue defects in immunotherapeutic agent (IA) directed to the treatment of these biomarker tissue defects in reestablishing tissue integrity, innate immunity, and associated adaptive immunity and disease resolution 329 studies of efficacy with acknowledgement of tissue deficits

26,906 studies of efficacy of the composition chemical moiety components (without active acknowledgement of tissue deficits) in the immunotherapeutic agent (IA) directed to the treatment of these biomarker tissue defects in reestablishing tissue integrity, innate immunity, and associated adaptive immunity and disease resolution

Biomarkers Tissue Deficits and Therapeutic Targets for Autoimmune Disease

Autoimmune disease nutritional deficiency 550 studies, compared with:

The chemical moiety component to detect biomarker tissue defects in the immunotherapeutic agent (IA) directed to the treatment of these biomarker tissue defects in reestablishing tissue integrity, innate immunity, and associated adaptive immunity and disease resolution 13,178 studies of efficacy with acknowledgement of tissue deficits

253,869 studies of efficacy of the chemical moiety components (without active acknowledgement of tissue deficits) in the subject immunotherapeutic agent (IA) directed to the treatment of these biomarker tissue defects in reestablishing tissue integrity, innate immunity, and associated adaptive immunity and disease resolution

The subject composition immunotherapeutic molecular embryologic amino acid profile for templating the proteins necessary for activation of the stem cell.

Biomarkers Tissue Deficits and Therapeutic Targets for Cancer

Cancer nutritional deficiency 6,901 studies, compared with:

The chemical moiety component to detect biomarker tissue defects in IPI's immunotherapeutic agent (IA) directed to the treatment of these biomarker tissue defects in reestablishing tissue integrity, innate immunity, and associated adaptive immunity and disease resolution 91,436 studies of efficacy with acknowledgement of tissue deficits

1,224,402 studies of efficacy of the chemical moiety components (without active acknowledgement of tissue deficits) in the subject immunotherapeutic agent (IA) directed to the treatment of these biomarker tissue defects in reestablishing tissue integrity, innate immunity, and associated adaptive immunity and disease resolution.

### Biomarkers Tissue Deficits and Therapeutic Targets for Decubitus Ulcer

Decubitus ulcer nutritional deficiency studies, 162 compared with:

The chemical moiety component to detect biomarker tissue defects in the subject composition immunotherapeutic agent (IA) directed to the treatment of these biomarker tissue defects in reestablishing tissue integrity, innate immunity, and associated adaptive immunity and disease resolution 1,678 studies of efficacy with acknowledgement of tissue deficits

1,486 studies of efficacy of the chemical moiety components (without active acknowledgement of tissue deficits) in the subject immunotherapeutic agent (IA) directed to the treatment of these biomarker tissue defects in reestablishing tissue integrity, innate immunity, and associated adaptive immunity and disease resolution

Biomarkers Tissue Deficits and Therapeutic Targets for Diabetes

Diabetes nutritional deficiency studies, 3,749 compared with:

The chemical moiety component to detect biomarker tissue defects in the subject composition immunotherapeutic agent (IA) directed to the treatment of these biomarker tissue defects in reestablishing tissue integrity, innate immunity, and associated adaptive immunity and disease resolution 9,626 studies of efficacy with acknowledgement of tissue deficits

195,960 studies of efficacy of the chemical moiety components (without active acknowledgement of tissue deficits) in the subject immunotherapeutic agent (IA) directed to the treatment of these biomarker tissue defects in reestablishing tissue integrity, innate immunity, and associated adaptive immunity and disease resolution

Biomarkers Tissue Deficits and Therapeutic Targets for Hypertension

Hypertension nutritional deficiency studies, 1,821 compared with:

The chemical moiety component to detect biomarker tissue defects in the subject composition immunotherapeutic agent (IA) directed to the treatment of these biomarker tissue defects in reestablishing tissue integrity, innate immunity, and associated adaptive immunity and disease resolution 5,320 studies of efficacy with acknowledgement of tissue deficits.

125,504 studies of efficacy of the chemical moiety components (without active acknowledgement of tissue deficits) in the subject immunotherapeutic agent (IA) directed to the treatment of these biomarker tissue defects in reestablishing tissue integrity, innate immunity, and associated adaptive immunity and disease resolution

Biomarkers Tissue Deficits and Therapeutic Targets for IBD

Crohn's Disease nutritional deficiency studies, compared with:

The chemical moiety component to detect biomarker tissue defects in the subject immunotherapeutic agent (IA) directed to the treatment of these biomarker tissue defects in reestablishing tissue integrity, innate immunity, and associated adaptive immunity and disease resolution studies of efficacy with acknowledgement of tissue deficits

5,334 studies of efficacy of the chemical moiety components (without active acknowledgement of tissue deficits) in the subject immunotherapeutic agent (IA) directed to the treatment of these biomarker tissue defects in reestablishing tissue integrity, innate immunity, and associated adaptive immunity and disease resolution.

Ulcerative Colitis nutritional deficiency studies, compared with:

The chemical moiety component to detect biomarker tissue defects in the subject immunotherapeutic agent (IA) directed to the treatment of these biomarker tissue defects in reestablishing tissue integrity, innate immunity, and associated adaptive immunity and disease resolution

4,784 studies of efficacy with acknowledgement of tissue deficits studies of efficacy of the chemical moiety components (without active acknowledgement of tissue deficits) in the subject immunotherapeutic agent (IA) directed to the treatment of these biomarker tissue defects in reestablishing tissue integrity, innate immunity, and associated adaptive immunity and disease resolution

Immunotherapeutics,

This synthetic biopharmaceutical comprises and provides the profiles, pathways and systems, and associated pre-clinical and clinical data. The hydrophilic surfactant such as phospholipid phosphatidylcholine activity of one of the key molecular components has been chemically engineered to significantly enhance penetration and distribution delivery of this therapeutics whether administered systemically (i.e. intravenously or orally) or transdermally (locally). Concurrently, therapeutically active in bio-robotically-self vesiculating the essence of cell membrane.

The successful integration of extensive clinical experience to include physiological chemistry, biophysics, molecular embryology, pharmacology to IPI's bio-molecular medical synthetic biopharmaceutical replication of the Chemical Engineering of Human and other Mammalian Tissue underlying biologic processes which serves as the basis for new drug discovery. This also disclosed in U.S. Pat. No. 6,974,796B1, which is hereby incorporated by reference in its entirety, and related U.S. and foreign applications.

10,119 studies were carried out, as a research directed collation using the five biochemical groups, 1-Amino Acids and glycine specified by the genetic code of human and mammalian tissue, phospholipid phosphatidylcholine, the essence of cell membrane, extra cellular matrix glycoprotein, and vitamins and minerals. 10,119 studies were carried out without any indication of correlation in accord with cell biology systems with foregoing molecular structures that underlie these cell biology systems; protein synthesis and molecular embryology tissue requirements.

Similar deficiency patterns and celiac disease like permeability leaks have been found in schizophrenia (6)

69 studies with regard to schizophrenia and abnormal gluten immune response similar to celiac disease

More than 800 studies of micronutrient deficiency patterns similar to IBD Crohn's and ulcerative colitis in diabetes mellitus (type 2)

Biomarkers Tissue Deficits and Therapeutic Targets for Lupus

Lupus nutritional deficiency 248 studies, compared with:

The chemical moiety component to detect biomarker tissue defects in the subject immunotherapeutic agent (IA) directed to the treatment of these biomarker tissue defects in reestablishing tissue integrity, innate immunity, and associated adaptive immunity and disease resolution 2,461 studies of efficacy with acknowledgement of tissue deficits.

42,893 studies of efficacy of the chemical moiety components (without active acknowledgement of tissue deficits) in the subject immunotherapeutic agent (IA) directed to the treatment of these biomarker tissue defects in reestablishing tissue integrity, innate immunity, and associated adaptive immunity and disease resolution.

Biomarkers Tissue Deficits and Therapeutic Targets for Pain Management

Pain management nutritional deficiency 164 studies, compared with:

The chemical moiety component to detect biomarker tissue defects in the subject composition immunotherapeutic agent (IA) directed to the treatment of these biomarker tissue defects in reestablishing tissue integrity, innate immunity, and associated adaptive immunity and disease resolution 2,839 studies of efficacy with acknowledgement of tissue deficits.

17,873 studies of efficacy of the chemical moiety components (without active acknowledgement of tissue deficits) in the subject immunotherapeutic agent (IA) directed to the treatment of these biomarker tissue defects in reestablishing tissue integrity, innate immunity, and associated adaptive immunity and disease resolution.

Biomarkers Tissue Deficits and Therapeutic Targets for Wound Healing

Wound healing nutritional deficiency 581 studies, compared with:

The chemical moiety component to detect biomarker tissue defects in the subject immunotherapeutic agent (IA) directed to the treatment of these biomarker tissue defects in reestablishing tissue integrity, innate immunity, and associated adaptive immunity and disease resolution 2,145 studies of efficacy with acknowledgement of tissue deficits

55,017 studies of efficacy of the chemical moiety components (without active acknowledgement of tissue deficits) in the subject immunotherapeutic agent (IA) directed to the treatment of these biomarker tissue defects in reestablishing tissue integrity, innate immunity, and associated adaptive immunity and disease resolution

Biomarkers Tissue Deficits and Therapeutic Targets for Severe Burns

Decubitus ulcer nutritional deficiency 63 studies, compared with:

The chemical moiety component to detect biomarker tissue defects in the subject composition immunotherapeutic agent (IA) directed to the treatment of these biomarker tissue defects in reestablishing tissue integrity, innate immunity, and associated adaptive immunity and disease resolution 91 studies of efficacy with acknowledgement of tissue deficits

2,384 studies of efficacy of the chemical moiety components (without active acknowledgement of tissue deficits) in the subject immunotherapeutic agent (IA) directed to the treatment of these biomarker tissue defects in reestablishing tissue integrity, innate immunity, and associated adaptive immunity and disease resolution

Biomarkers Tissue Deficits and Therapeutic Targets for Cardiac Arrhythmia

Cardiac arrhythmia nutritional deficiencies, 496 studies compared with:

The chemical moiety component to detect biomarker tissue defects in the subject composition immunotherapeutic agent (IA) directed to the treatment of these biomarker tissue defects in reestablishing tissue integrity, innate immunity, and associated adaptive immunity and disease resolution 1,137 studies of efficacy with acknowledgement of tissue deficits

29,604 studies of efficacy of the subject chemical moiety components (without active acknowledgement of tissue deficits) in the subject immunotherapeutic agent (IA) directed to the treatment of these biomarker tissue defects in reestablishing tissue integrity, innate immunity, and associated adaptive immunity and disease resolution

Biomarkers Tissue Deficits and Therapeutic Targets for Parkinsonism

Parkinsonism nutritional deficiency 134 studies, compared with:

The chemical moiety component to detect biomarker tissue defects in the subject composition immunotherapeutic agent (IA) directed to the treatment of these biomarker tissue defects in reestablishing tissue integrity, innate immunity, and associated adaptive immunity and disease resolution 587 studies of efficacy with acknowledgement of tissue deficits

48,903 studies of efficacy of the chemical moiety components (without active acknowledgement of tissue deficits) in the subject immunotherapeutic agent (IA) directed to the treatment of these biomarker tissue defects in reestablishing tissue integrity, innate immunity, and associated adaptive immunity and disease resolution.

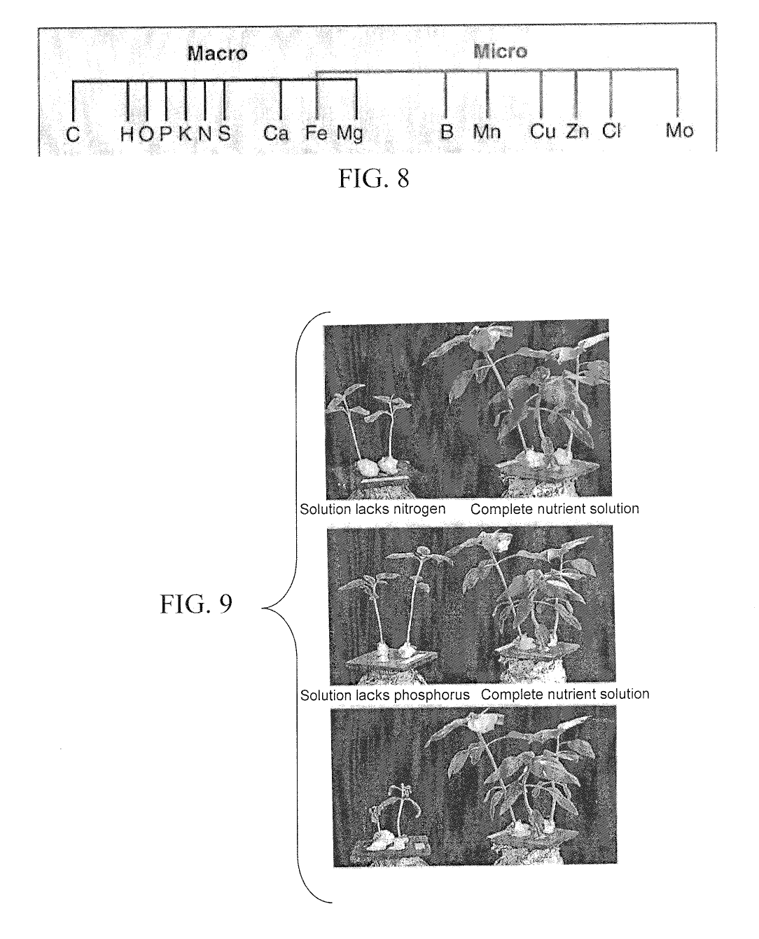

“Beneficial nutrients are another category of elements taken up by plants. Beneficial nutrients either are required for or enhance the growth of a particular plant. Horsetails require silicon as a mineral nutrient and sugar beets show enhanced growth in the presence of sodium. Nickel is a beneficial mineral nutrient in soybeans when root nodules are present. Aluminum is used by some ferns, and selenium, which is often fatally poisonous to livestock, is used by locoweeds. Approximately 95% of a typical plant's dry weight (weight excluding free water) is carbon, hydrogen, and oxygen. These are the elements that are found in most organic compounds, such as carbohydrates. Carbon dioxide (CO2) supplies carbon, and water (H2O) supplies hydrogen and oxygen found in the organic compounds of a plant. Essential nutrients for plants include carbon, hydrogen, oxygen, phosphorus, potassium, nitrogen, sulphur, calcium, magnesium, iron, boron, manganese, copper, zinc, choline and molybdenum”. (Biology, Mader, McGraw Hill, N.Y., 2007, pp 460-61). (See FIG. 8).

Periodic Table Components of Human and Mammalian tissue where the majority have an atomic number of less than 30 with additional trace elements (such as Molybdenum and Iodine which have masses of 42 and 53 respectfully). In contrast, the heavy metals that are not present in the body normally are such as gold at 79 (with the exception of dental fillings, so resistant to corrosion) and lead at 82 (in the case of toxicity as a toxic metal). (See FIG. 9).

Some of the available tests include: protein serum Albumin, Homocysteine, B vitamins associated (6, 12 in the form of methylcobalamin also referred to as methyl B12, folic acid in the foils of methylfolate) with disruption of cysteine and methyanine) as well as offering B1 (thiamine), B2 (riboflavin), and B3 (niacin) levels, as well as vitamin D3.

Resveratrol, Garlic (anti-fungal)>40 studies, Soy (check reference)>60 studies, Curcumin (5-10 studies). Green tea (5-10 studies), Cinnamon, Onion has similar properties, Echinacea, Aloe Vera, and probiotics such as lactobacillus acidoph such lactobacillus bifidus.

Regarding tissue integrity, innate immunity, and GRAS items, to further reinforce innate immunity and prevent disrupted innate immunity as discussed as further clinically in the Japanese report allergic disease with its type 2 T-helper cell innate immunity, significantly favoring a co-existent sarcoid with the co-existence makes sarcoid a much milder disease without the characteristic features of T-helper cell 1 of every disrupted immunity with a granulomatous disease such as tuberculosis without a known cause, similar to this inventors' observations pertaining to subject composition in that in my first year of practice in practicing allergy and immunology, a boarded subspecialty of internal medicine and pediatrics, I only saw one case of cancer weekly as opposed to in the internal medicine practice. Defensins belong to the family of antimicrobial peptides that play a central role in innate immunity in all species of plants. We have previously reported the purification of antimicrobial peptides from Scots pine seedlings and the identification of some of them, including defensin, by mass spectrometry (Recombinant expression, affinity purification and functional characterization of Scots pine defensin 1. Kovaleva V, Krynytskyy H, Gout I, Gout R. Plants produce a variety of molecules to defend themselves from fungal pathogens).
i. Tissue integrity to innate immunity: Alternate pathway would be the adaptogens of the plant, representing defensins protecting from fungal infection and infestation=innate immunity. Adapt adaptogen of the plant. It is believed that this represents some index of significance in the bioscientific world of these components in therapeutics.
   These observations were prompted by the fact that some patients have a fungal infection on only one foot and their predominant associated disease is the gout more commonly associated with one foot over the over, with chronic arthritis brought about by the gout. Just as the plants have responded to the fungal toxins or micro-toxins, this provides an integrated continuity of forefront of therapeutic approach. This can be exemplified by the splinter hemorrhages that clinically have been observed to be associated with a low grade septicemia and the presentation in this patent of the addition of garlic capsule (1, 2-3 times daily) has resulted in disease resolution and the prevention of the recurrence of these hemorrhages that are thought to play a role in arthrosclerosis and coronary artery disease, with an illustration of a patient with which this was applied. Therefore, in this case, having maximized tissue integrity normalization innate immunity by adapting the adaptogen of the plant, we provide the addition of innate immunity derived from the plant.
ii. The fungi and the microtoxins derived therefrom were presented internationally:
   CHESSER-NAUGLE International Lectureship Grant:
   The Fungal/Mycotoxin Connections:
   Autoimmune Diseases, Malignancies,
   Athero-sclerosis, Hyperllpldemias, and Gout
   Oct. 11, 1993
   A. V. Costantini, M.D.
   [Dr. A. V. Costantini, MD is the Director of the World Health Organization (WHO) Mycotoxin Collaborating Center at the University of Freiburg, Germany. Prof Dr. A. V. Costantini, MD was the keynote speaker at the 1993 conference of the American Academy of Environmental Medicine held in Reno, Nev.]

Twenty-Eighth Annual Meeting New Horizons in Chemical Sensitivities: State of the Art Diagnosis and Treatment

The Fungal/Mycotoxin Etiology of Malignancies and Auto-Immune Diseases
The vast majority of malignancies and all of the auto-immune diseases are of unknown cause. Fungi/mycotoxins have been for the most part ignored as documented cause of many malignancies and of auto-immune diseases.

The Fungal/Mycotoxin Etiology of Atherosclerosis and Hyperlipldemia
Atherosclerosis and hyperlipidemia are clinical entities of previously unknown etiology. Fungi & their toxins have been ignored as documented etiology of both entities. Hyperlipidemia is induced by a number of mycotoxins. Seasonal variations in hyperlipidemia correlates to seasons of maximal fungal growth and mycotoxin production.

Fungal/Mycotoxin Etiology of Gout & Hyperuricemia
Gout and hyperuricemia are clinical entities of previously unknown etiology. Fungi/mycotoxins have been ignored as documented cause of both entities. The etiopathogenetic mechanisms are not the usual patterns of invasive-type mycoses norof mycotoxicoses, but incorporate occult features of both of these mechanisms resulting in abnormal biochemical findings

It is believed that if we were to establish spreadsheets for this chemical moiety of tissue integrity, this int run would be providing the innate immunity and adaptive immunity for disease resolution. It is based on these observations that the other diseases we have found success through subject composition disease resolution including the inclusion of GRAS items including from plants innate immunity, a product of defensins and adaptogens, particularly if we include the plants adaptogen defensin innate immunity.

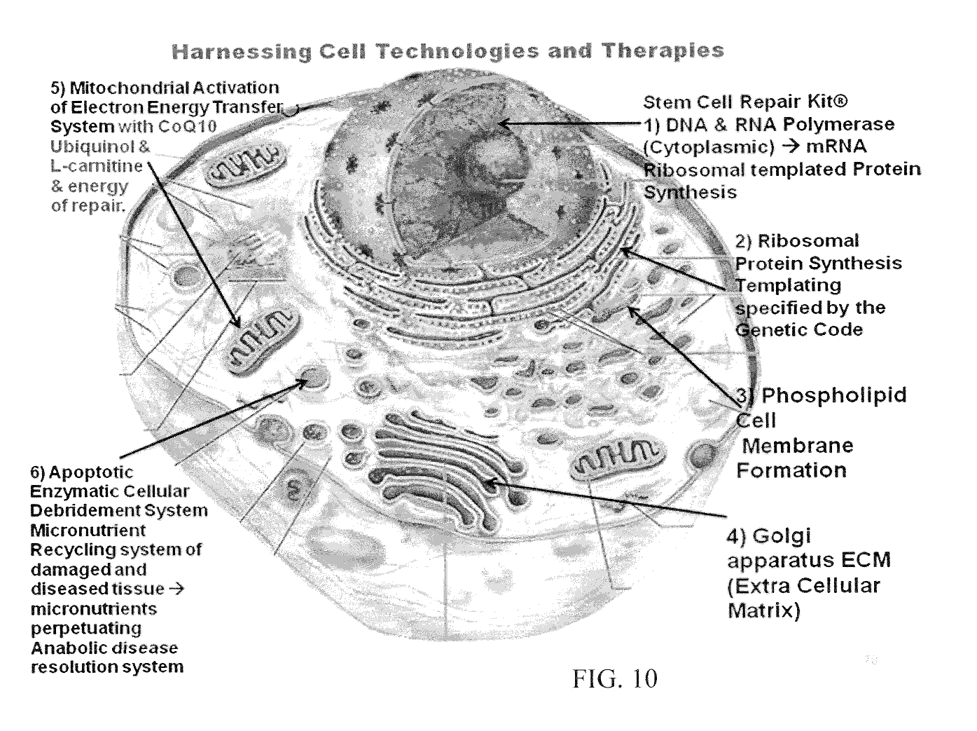

### Stem Cell Repair Kit® (See FIG. 10).

This broad-based regenerative medicine stem cell activation healing system based on molecular structures that underlie key cell biology systems inclusive of molecular embryology therefore the broad based efficacy that has been anticipated with the natural embryonic stem cell is being replicated as asynthetic biopharmaceutical using components equivalent to human tissue that also replace documented deficiencies in human tissue and the cell cycle by providing a composition that mimics human tissue from a biological and cell biology system and cell cycle vantage point without any known undesirable effects

Progressive activation of the cell and stem cell tissue assembly system, inactivated by diseased tissue. The subject therapeutic use in averting the need for major organ transplantation including averting full thickness skin graft. Averting the need for joint prosthesis in activating this cell & stem cell tissue assembly system, utilizing molecular embryologic technology of organogenesis in restructuring major organ systems applied here to the stem cells found at the distal stem cell end of the bronchiolar system in asthma, the distal stem cell end of the ductules in the liver and the distal stem cell end of the kidney tubules at the site of Bowman's capsule. This includes 347 clinical responses, 60 transdermal lesional applications: 27 preclinical veterinary care, plus 33 clinical studies of resolution of arthritis sports medicine trauma in-vivo. 71 in-vitro studies of human tissue in disease resolution, reversal, transdermal lesional, oral, I.V. administration. >100 wound healing studies.

Depletion of Ubiquinol and ubiquinone can induce mitochondrial dysfunction, critically decreasing energy production and aerobic capacity required for normal heart function.

Cells convert nutrients into an energy-rich molecule called ATP; Ubiquinol provides the spark that ignites the reaction: more than 90% of ATP in the body is produced in the mitochondria.

The enzymatically driven conversion of Ubiquinol to ubiqunone facilitates the critical transfer of electrons in the mitochondrial electron transport chain.

As well as energy production, CoQ10 as Ubiquinol acts as an antioxidant to protect heart cell mitochondria from free radical damage.

Promising trials and studies involving Ubiquinol include: Cardiovascular Disease; Huntington's Disease; Parkinson's Disease; Down's Syndrome; Diabetes; Aging; Mitochondrial Disease; Sport's Fitness; and Fatigue.

The following is a quote from an eminent scientist, a director of a laboratory dedicated specifically to multiple myeloma, who viewed a recent scientific exhibit in Cambridge Mass., summarizes the subject Maximizing and Optimizing the Efficacy and Safety of IPI's Immunotherapeutic Agent.

“While I attended a conference in Boston organized by Cambridge Healthtech Institute, I met Dr. Leonard Girsh. Dr. Girsh has developed a revolutionary immunotherapeutic agent (IA) that promotes apoptosis of cancer cells and without known side effects can reestablish normal cell cycle. Dr. Girsh informed me that the IA agent is non-antigenic and could be used as a ‘drug’ or as a supplement* to present treatment remedies to hasten cancer cure*. Dr. Girsh successfully used the IA in 256 cases. He was able to reverse diseases with poor prognosis, e.g. averting the need for organ and skin transplantation and other major surgeries.”

The Foregoing Best Explains the Chemical Moiety (Approved by the Regulatory Affairs Agency) for this Conference:

However the subject composition also contains additional components. To our knowledge, nobody has included in a single treatment plan all of the components described in the orphan drug designation request for the subject compositions. Our hypothesis is that the elemental diets discussed in the published literature and described in the designation request were “only” as effective as steroids because the elemental diets were not provided in the optimal molar ratio, and did not include important additional components that are necessary for cellular function and regeneration. In particular, elemental diets lack the components of the cell membrane and extracellular matrix that are provided in the subject compositions. By supplying the body with the chemical components of the extracellular matrix, such as cartilage containing chondroitin sulfate and collagen, tissue repair and anti-inflammatory activity is enhanced since the extracellular matrix provides tissues with structural support and directs cell migration, attachment, and organization. Cell membrane formation and repair is enhanced by the simultaneous administration of phospholipids, essential fatty acids, and EPA. In sum, the compositions of the subject invention are designed to repair diseased human tissue and enhance the formation of healthy tissue. Since all of the components are necessary for cellular function, all of the moieties in the compositions are thought to be essential and “active.” We discussed these other moieties in the designation request, and provided references for many of the components that are not already part of the elemental diets described above. Additional references can be provided on request.

Section 5(b)(3) of the FDA Orphan Drug Act: The term “medical food” means a food which is formulated to be consumed or administered internally under the supervision of a physician and which is intended for the specific dietary management of a disease or condition for which distinctive nutritional requirements, based on recognized scientific principles, are established by medical evaluation.

Wound Healing and Tissue Repair in Adults

Trace element supplementation after major burns increases burned skin trace element concentrations and modulates local protein metabolism but not whole-body substrate metabolism.

BACKGROUND: After major burns, patients exhibit an intense catabolism, and the wounds often require surgery and grafting for closure. Complications, such as weight loss and delayed wound healing, are worsened by trace element (TE) deficiencies. OBJECTIVE: We aimed to assess the effects of TE supplements on systemic substrate turnover and local protein metabolism during wound healing after major burns.

DESIGN: This was a prospective, randomized, placebo-controlled trial in 21 patients aged 35+/−11 Y with burns on 45+/−16% of their body surface area; 12 had skin biopsies performeded on days 3, 10, and 20, and 10 patients underwent a stable-isotope investigation on day 10. Intravenous copper, selenium, and zinc (TE group) or vehicle (V group) was given with a saline solution for 14-21 d. On day 10, [(13)C]phenylalanine (600-mug/kg bolus followed by 12 mug. kg(−1). min(−1)) plus 6-[(2)H(2)]glucose and [(2)H(5)]glycerol were infused for 6 hrs. to determine skin protein turnover. Biopsies were performed 1 and 6 hrs. after the start of infusion to determine [(13)C]phenylalanine enrichment.

RESULTS: The patients' mean ages and burn severity did not differ significantly between the groups or between the skin investigations subgroups. Plasma TE concentrations were significantly higher in the TE group. In the burned areas, the skin contents of selenium (P=0.02) and zinc (P=0.03) increased by day 20. The supernatant-to-plasma (13)C enrichment ratio in burned skin was 0.363+/−0.094 (TE group) and 0.286 0.130 (V group) after 1 hy. (NS) and 0.592+/−0.153 (TE group) and 0.262+/−0.171 (V group) after 6 hrs., which reflected lower catabolism in the TE group (P=0.03). No significant differences in whole-body substrate turnover were found between the groups.

CONCLUSION: TE supplementation was associated with an increased skin tissue content of selenium and zinc and with a reduction in skin protein catabolism.

With an interdependent signaling system analogous to a present day computer with the subject IA also based on bio-mimetic as a micronutrient bioenvironmental tissue equivalent. This is also based on these components as a composition of tissue and continuum of this interdependent signaling system also analogous to a present day computer while at the same time insuring the adequacy of supporting and maintaining the composition of tissue. The indications for the multiplicity of disease management that this offers in accordance with out spreadsheet documentation. This is exemplified by inflammatory bowel disease management, as an introduction to the indications for the management of this disease based on thousands of publications, however, none of the publications composites what we believe to be the most complete tissue equivalent available.—Since each publication of the thousands of publications represent one or at least a few of these tissue components as opposed to the tissue entirety, which has been our goal here as a composition of matter immunotherapeutic agent (IA).

These components represent about 20% of the periodic table elements and remarkably are only the very light elements based on atomic mass of these elements. These are the elements of human tissue mimicked here in the subject immunotherapeutic agent (IA) (equivalent to the cytoplasmic tissue sap as observed in my initial publication of tissue engineering in the modified (vagal crush) surgical procedure of Vagotomy reserved for severe complications of a peptic ulcer.)

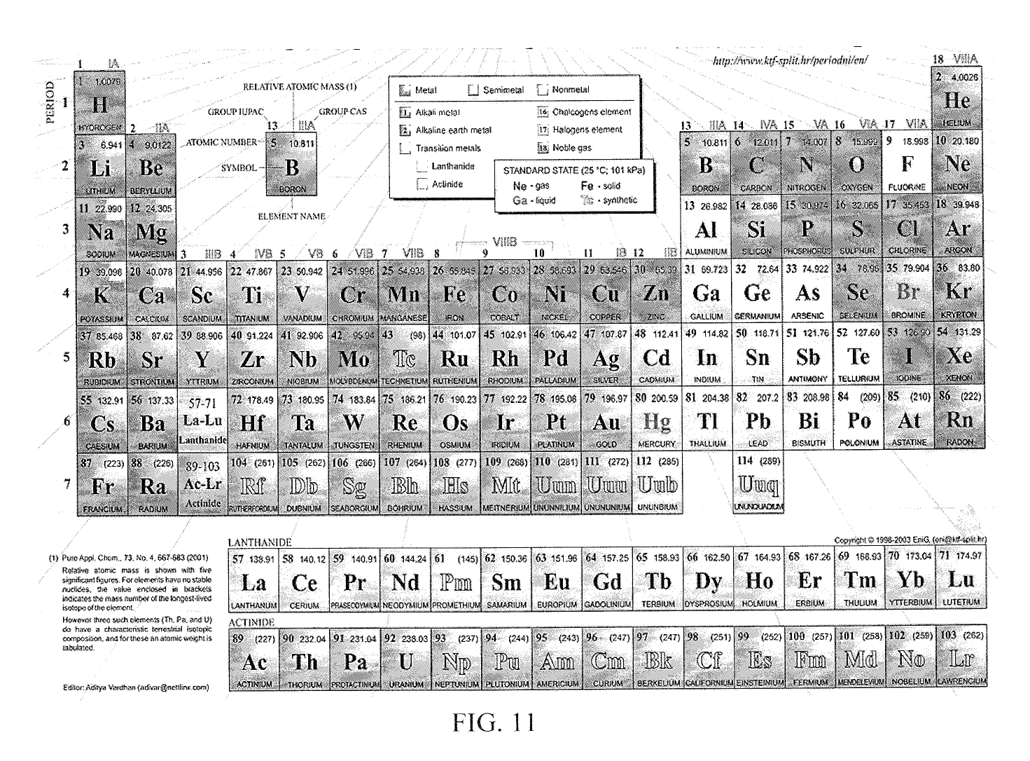

Periodic Table of the Elements in Human and Mammalian Tissue Demarcated 21 Elements that Biochemically Describe Human Tissue and its Composition. (See FIG. 11).

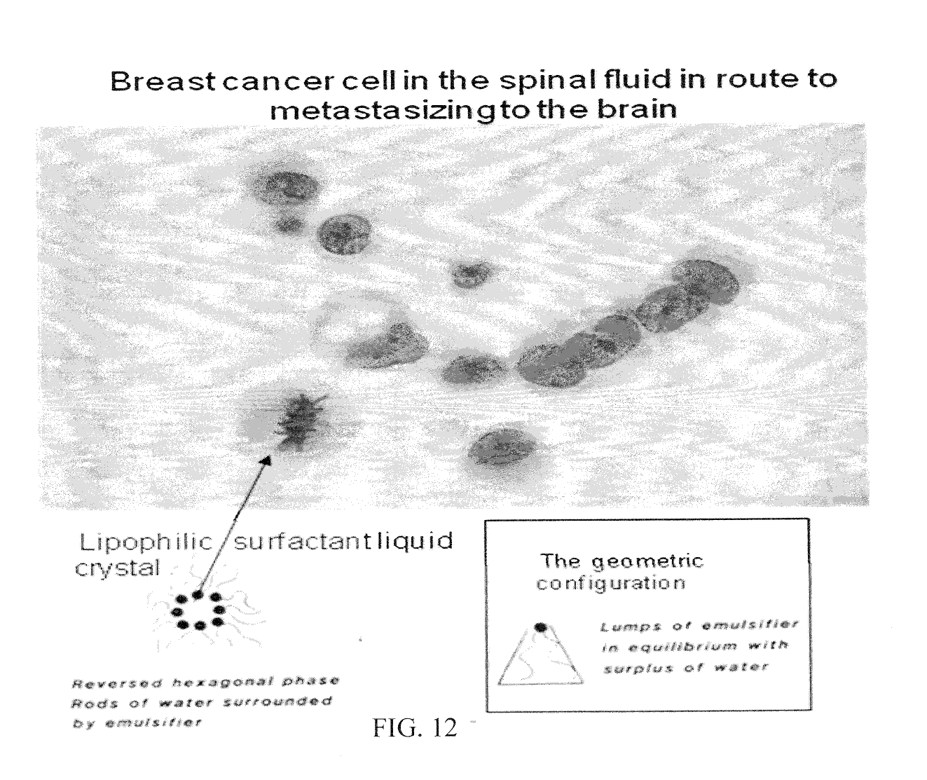

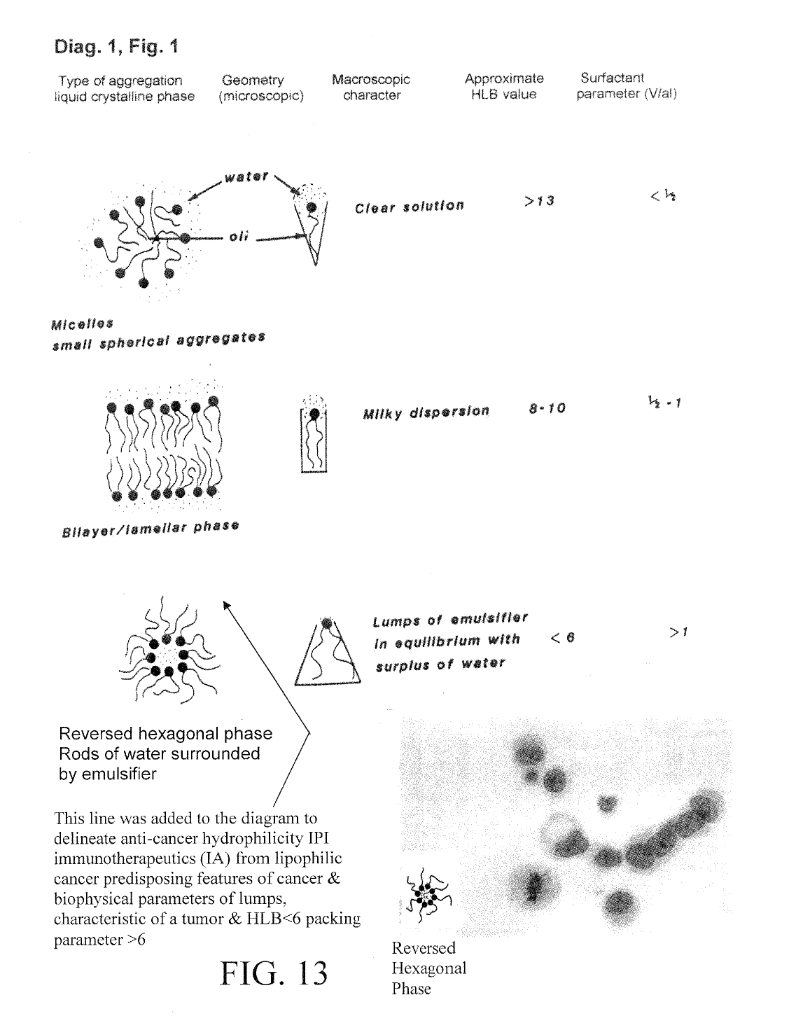

(See FIGS. 12, 13).

All these micelles are liquid crystals, and the hydrophilic HLB of greater than 8-10 is compatible with anti-cancer therapeutic, ideally the higher the HLB, the greater the anti-cancer activity which is the activation of apoptosis and imposing a normal cancer cell kill on these cells otherwise scheduled to die but have been resistant to lysosomal surfactant surface-to-surface enzyme substrate activating the normal apoptotic system of cancer cell death. This normalizes the deficiency of the cancer cell: an inability to proceed to scheduled cell death, normally for example all the cells against the gastro-intestinal tract and our white cells are scheduled to live only 2-6 days.

In neurologic disease, this same principle of activation of the lysosomal surfactant surface-to-surface activity in regard to preventing a build-up of catabolic neural tissue in neurologic disease such as Parkinson's disease, Alzheimer's disease, and congenital neurologic enzyme deficiency diseases such as Gaucher's disease and Niemann-Pick disease. By this therapeutics it is believed that small diffusible metabolizable end products are produced in contrast to the cell cycle being stuck with non-metabolizable products. To maintain the ideal normal homeostatic balance (best seen in youth), we must have enabolism counterbalancing metabolism, and we must have all of the systems of the cell cycle, including the lysosomal system, which is in waste management of metabolism products preventing any build-up or jam of the cell cycle by normalizing the surface-to-surface interaction of enzyme and protein as well as providing for adequate proteomic production and supply of protein from the activated templated ribosomal protein production system. This is analogous to a smoothly running factory, including production as the cell tissue assembly system (see cell cycle diagram) and waste disposal system of worn and damaged tissue as metabolizable products that can be recycled and added to and reinforcing IA subject composition as well as the energy supply system of the mitochondria (this belief is a product of an integration of the basic preclinical sciences of medicine).

This is a component of normalizing the cell cycle (see diagram of cell cycle) wherein by activation of normal protein templating the required protein enzymes are formed to address any of the deficiencies as well as the required polymerase enzyme protein as DNA and RNA polymerase to provide normal DNA and RNA structures preventing the patient, it is believed, in being stuck with their disease. See Whitehead Institute's quote of normalizing the micronutrient bioenvironment to enhance genomic expression by as much as 30%.

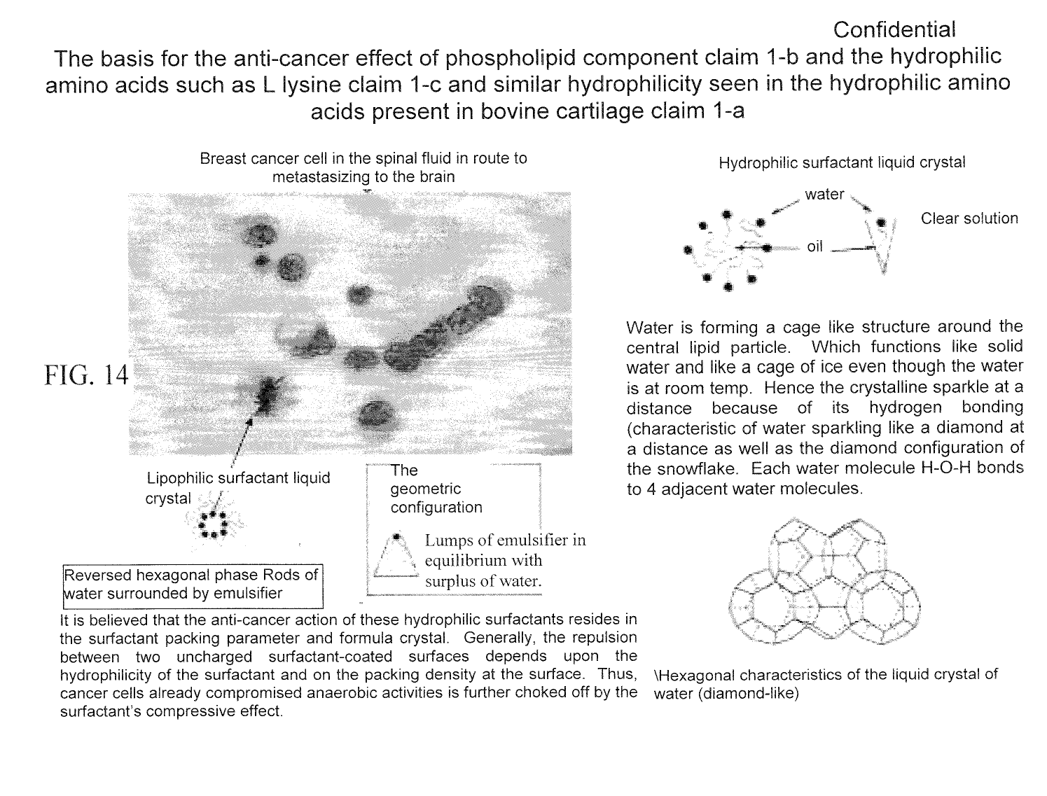

These all have been referred to as “storage diseases” but functionally it is believed they should be looked upon as a stuckage” (jammed) pathogenic phenomena, in that the normal metabolic flow of the cell cycle is arrested here. (see Parkinson's section). (See FIG. 14).

Most importantly, all these structures water molecules representing the majority of tissue, 50-60% hydrogen bond to 4 adjacent molecules by varying the length and bond of the hydrogen bonds. Most importantly, with regard to the hydrophilicity of these surfactants as an anti-cancer therapeutic normalizing domain is the fact that the non-polar lipid material normally present in about 25% or less is being caged inside this hydrophilic surfactant and this cage is referred to as a clathrate and depending on how great the hydrophilicity is the surfactant packing parameter will cage these non-polar fats in a central position to this clathrate cage-like structure.

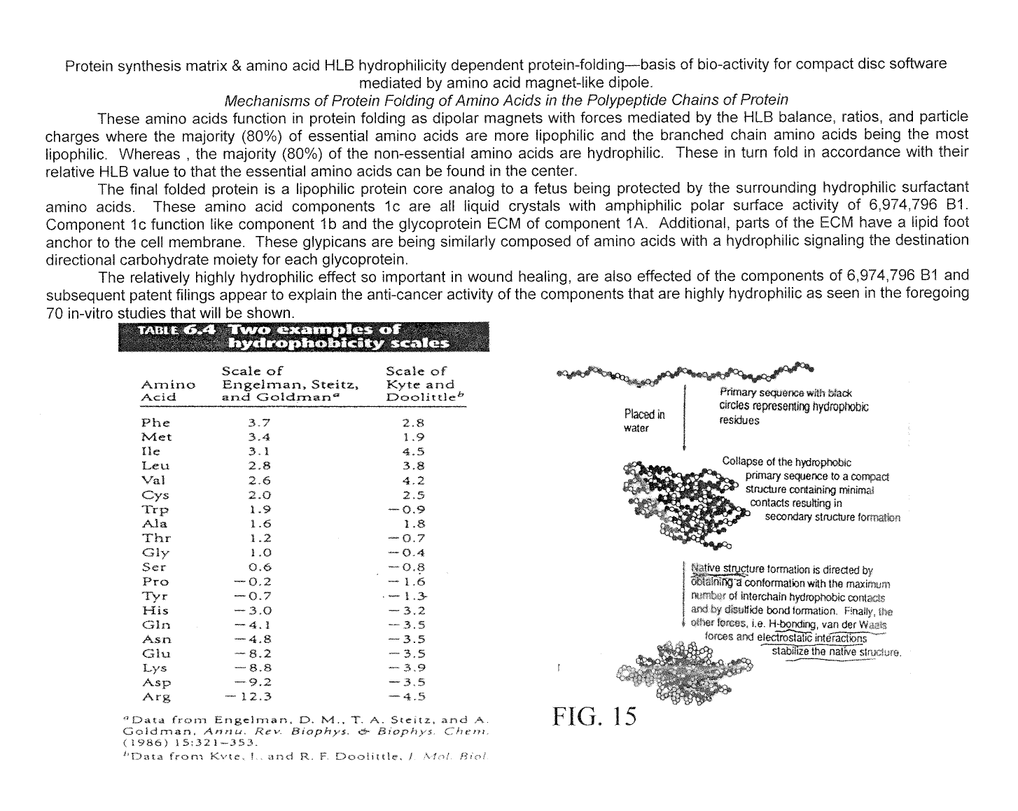

The basis of the biologic activity further based on the structure providing the biologic function of protein. As in the case of the cell membrane, the water-loving soluble component represents the outer coating of the cell as well as the outer coating of the biologically functioning protein, with the fat-loving portion folded up in the center like a fetus. (See FIG. 15).

The following 67 in vitro studies of cancer cells exposed to hydrophilic compounds illustrate degrees of surfactant hydrophilicity and the associated cancer cell kill of 70-100% of the cancer cells. The greater the hydrophilicity, the greater the cancer cell kill.

In contrast, 4 studies were done using lipophilic surfactants using a HLB of 2 such as PGPR (polyglyceryl polyricinolate) where there is an increase of cancer cells as much as 50% and with a lesser lipophilicity as much as 25%.

Ideally in treatment of cancer the natural surfactant of phosphatidyl choline is preferred. This is based on high HLB acceleration studies on activation of germination of grass seed in that when the synthetic surfactants are used the subsequent observation of the grass growth is that the grass is friable and not so with the use of the natural phosphatidyl choline surfactant.

The ribosomes of dry seeds are inactive, which contain, like sea urchin eggs, a store of masked mRNAs. When the seeds are soaked in water in order induce germination, the preformed mRNAs bind to the to ribosomes; polysomes are formed and protein synthesis begins.

However, it is not in the prior art that increasing the hydrophilicity accelerates germination of the seeds.

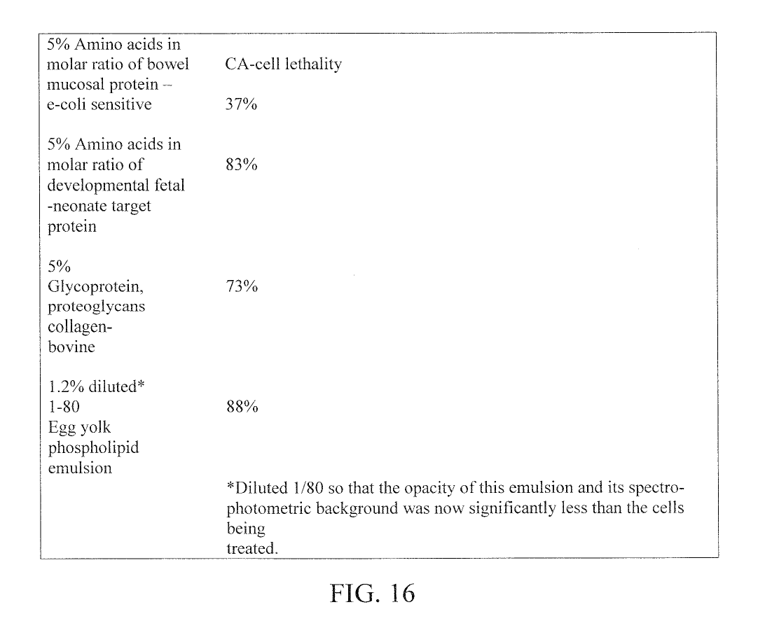

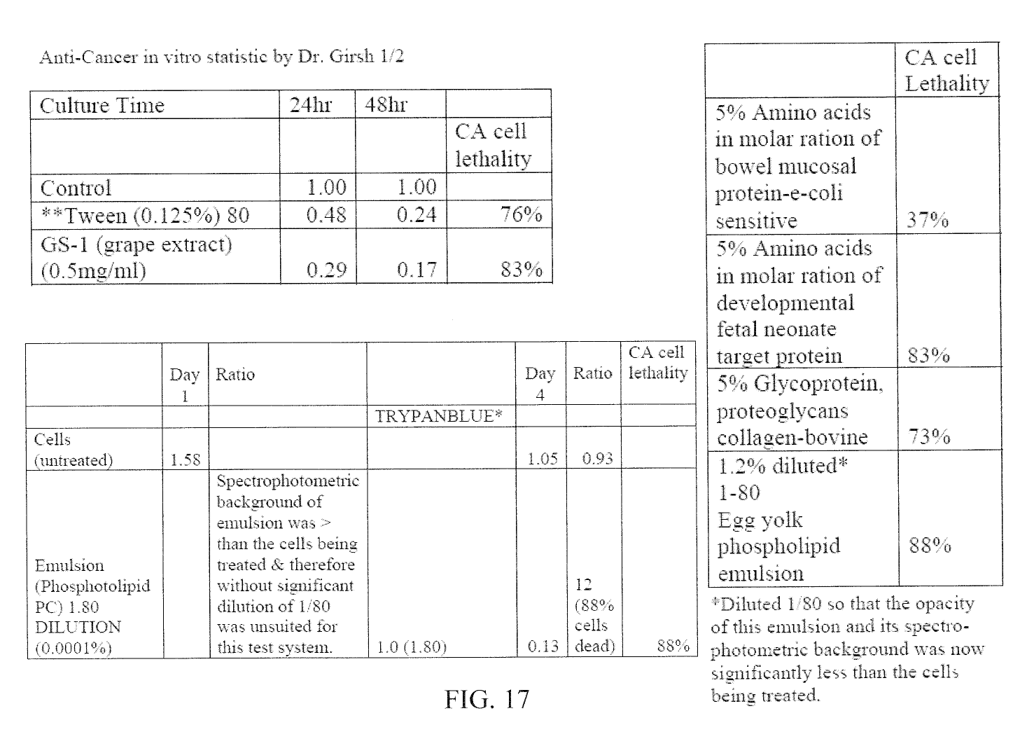

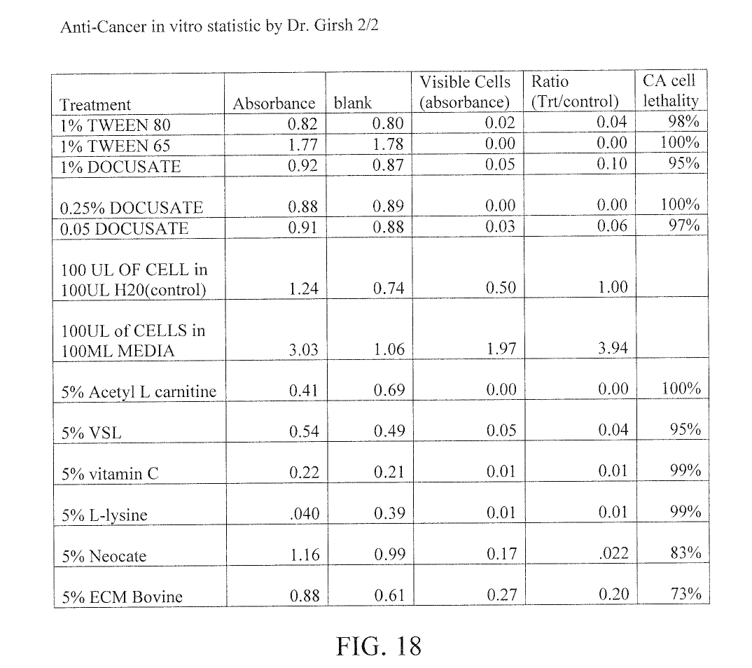

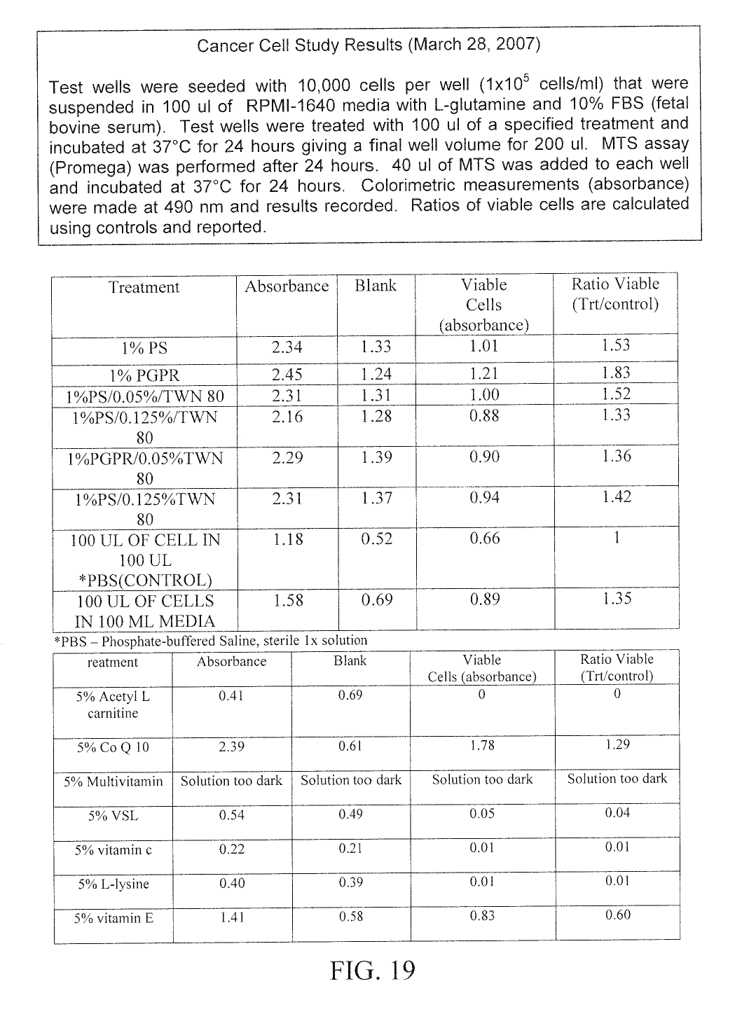

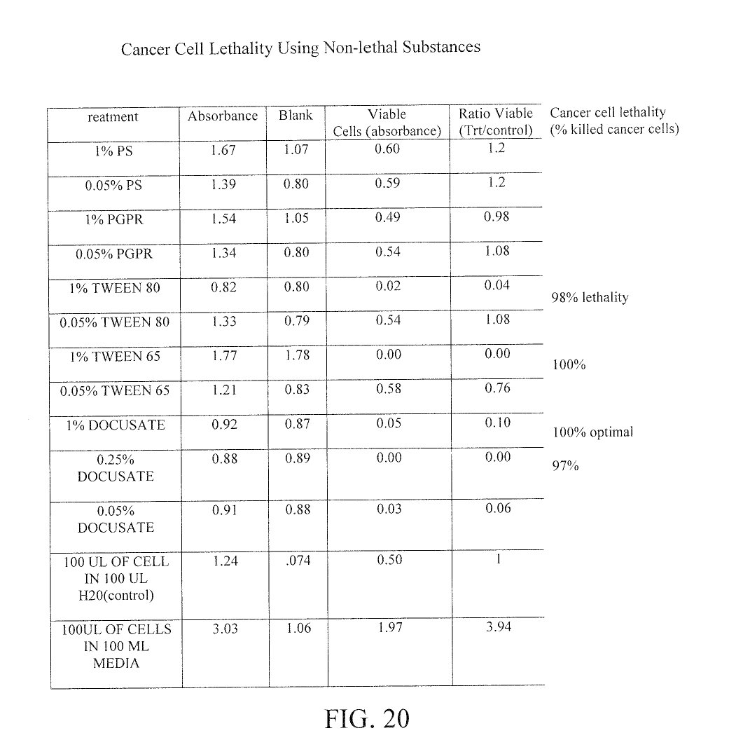

We have found by studies of activation of germination (with a measured effect of reducing the germination time by half, with a 3 day CHIA grass seed germination time is reduced to 1½ days, and with grass seeds otherwise, it was reduced from 7 days to 3 days, illustrating an acceleration of germination) of the grass seeds with hydrophilic surfactants that the use of naturally existent phosphatidyl choline egg yolk phospholipid diluted about 1:30 fold thereby activating the hydrophilicity more significantly than if undiluted. Whereas if we use a 1:80 dilution (dictated by the spectrophotometric studies) the cancer cell kill rate is 88% in 24-48 hours. (See FIGS. 16, 17, 18, 19, and 20).

This Colloidal & Emulsion Technology Domain, where Physics, Chemistry, Biology, and Technology Meet.

In the immunotherapeutic recomposition of tissue and tissue micronutrient defect as biomarkers to activate noninvasively the patient's own stem cells and the healing system (through these molecular structures that underlie molecular embryology) all free of known undesirable effects (preapproved as safe in the Code of Federal Regulations 21—FRC21 and GRAS Regulations).

The enhancement of micronutrient bioenvironment in enhancing the genomic expression to bring about significant potential reversibility to the genetic predisposition to disease. In activating the cell cycle and the proteomic display the DNA and RNA polymerases reform to proceed with mitosis and the next cell cycle and the activation of the ribosomal templating system deactivated by diseases.

Alloy: a substance that is a mixture, as by fusion, of two or more metals, or of a metal and something else.

Steel: a hard, tough metal composed of iron alloyed with various small percentages of carbon and often variously with other metals, as nickel, chromium, manganese, etc., to produce hardness, resistance to rusting, etc.

Neurogenerative Disease Tryptophan and melatonin to account for serotonin which is important neurotransmitter for pain and providing comfort. in pyrosine (amino acid chemical in terms of inflammation of neurotransmitters such as dopamine, nepanephrine).

These lysosomal (stuckage disease) of storage in a sense store these metabolic products pathologically and don't allow for the breakdown of components and therefore the hydrophilic surfactant would enhance the apoptosis additionally by enhancing the surface-surface interaction of substrate and enzyme (like fingers interlocking in a clasp). This stuckage state exists in neurological diseases such as Parkinson's and Alzheimer's associated with cognitive disease with nervous tissue breakdown that hasn't been recycled. With IA can be applied to the forehead and hairline and nasal cavity to treat the breakdown of nervous tissue.

We are activating normalcy in the same fashion as the I-ILB with the lewwy bodies just as we did with the in-vitro studies. Disease resolution is accomplished by reconstitution of tissue as we have done with arthritis, bone fractures, averting the need for a liver transplant.

In the case of these diseases it is looked at as dysfunctional hyperactive autophagy. The only similarity to cancer is the disruption of the lysosomal apoptotic system, where it is hyperactive and less discriminative as opposed to hypoactive.

This is the therapeutic direction and solution:

Modify the genetic architectural plan through the normalization of the micronutrient bioenvironment with enhancement of the genomic expression (Whitehead Institute, NIH publication, 2001) with, it is believed, normalization of the proteomic display of all protein enzymes but particularly the polymerases DNA and RNA. Therefore, it is to be expected that the lysosomal enzymes required for normal lysosomal apoptosis that we have shown have been activated by hydrophilicity of polar surface active lipid components of subject composition the degenerative disease, such as but not limited to dysfunctional lysosomal system such as Alzheimer's disease and Parkinsonism, and a dysfunctional lack of deficiency of apoptotic lysosomal, both of which we have found and believe are normalized and activated by the micronutrient bioenvironment of subject composition and its effect on normalizing cell tissue assembly system and the waste disposal lysosomal system and the bioenergetic mitochondrial system (see cell diagram, FIG. 10)

(Case 1.) Pain management of a male patient, age 82, presenting with prodromal symptoms of herpes with severe unilateral left ocular edema and conjunctivitis with vesicles on left side of forehead. A very common associated clinical finding of herpes zoster of the forehead up to the hairline. This was preceded by pain upon combing hair, also on the left side of head, one day prior to dermatologic involvement.

Transdermal cream of subject composition was applied to left scalp and left forehead was associated with clearance pain with particularly noteworthy absence of residual pain. Usually this is associated with poor prognosis in regard to the persistence of pain. Also, corneal damage was not noted in this patient with use of subject composition transdermally, (as presented here in case reports of reversal of arthritis and tendo-synovitis in 27 horses of equine species).

The treating ophthalmologist was happily surprised with this outcome. He did not feel the acyclovir topical 5% applied 3 times daily played a role. Nor did he feel that the acyclovir oral therapy 200 mg 3 to 4 times daily played definitive role seen with subject composition in completely addressing the very common residual complications of herpes zoster and normalizing neuronal impulse flow along with normalizing tissue structure and function. (one year follow-up)

Super Zeaxanthin (OptiLut®, LuteinPlus® and MZ® [Marigold (Tagetes erecta) extract (flower) (free lutein equivalent 10 mg)]38 mg, Zeaxanthin & Meso-zeaxanthin blend [micronized zeaxanthin, OptiLut®, LuteinPlus® and MZ® Marigold (Tagetes erecta) extract (flower)] 3.75 mg, C3G (Cyanidin-3-glucoside) [from European black currant (Ribes nigrum) extract (fruit)] 2.2 mg. Other ingredients: soybean oil, gelatin, glycerin, beeswax, purified water, lecithin, annatto, potato maltodextrin. Contains soybeans)) has been used in several patients in their late 70s and 80s that had visual dysfunction with helpful response in their vision improvement and Food Carotene (Solaray) has been suggested to the patient tin the form of foods such as carrots, for this natural form, with the avoidance of any synthetic beta-carotene.

The blackcurrant jelly component is helpful in improving night vision. I have used this treatment one time a day and suggested an increase to two times a day to a female patient aged 84 being treated for age-related macular degeneration by her ophthalmologist. Black currant jelly can improve night vision with 30 minutes of use, and the beta-carotene gives clear sharp vision. Lutein and Zeaxanthin help reduce glare, particularly bothersome at night. As Zeaxanthin helps with close and distance, maybe these efficacies can be used as a check list in tracking the improvement of a patient. To date, the patient treated (treated for about 4 weeks) for a condition she has had progressive for several years has not worsened, awaiting to see improvement.

See diagram for instrumentation and birefringence.

Progressive activation of the cell & stem cell tissue assembly system, inactivated by diseased tissue. The subject therapeutic use in averting the need for major organ transplantation including averting full thickness skin graft. Averting the need for joint prosthesis in activating this cell & stem cell tissue assembly system, utilizing molecular embryologic technology of organogenesis in restructuring major organ systems applied here to the stem cells found at the distal stem cell end of the bronchiolar system in asthma, the distal stem cell end of the ductules in the liver and the distal stem cell end of the kidney tubules at the site of Bowman's capsule. This includes 347 clinical responses, 60 transdermal lesional applications: 27 preclinical veterinary care, plus 33 clinical studies of resolution of arthritis sports medicine trauma in-vivo. 71 in-vitro studies of human tissue in disease resolution, reversal, transdermal lesional, oral, I.V. administration.

>100 wound healing studies.

### (See FIG. 10)

### Embodiments of Instrumentation to Examine Living Tissue Non-Invasively

Avoiding necessity for surgical biopsy and therefore minimizing any risk of surgical biopsy, as well as making it more economic. But most importantly to avoid surgical examination where surgery cold impose the risk of angiogenesis incorporating the freed cancer cells into the capillaries with the imminent risk of metastasis, the most feared and deadly risk of cancer. Several publications document this risk, several publications to follow.

A device wherein a non-invasive probe is utilized to obtain birefringence light signals and attached to examination probes inclusive of any endoscopy inclusive of ultrasound and ultrasound endoscopy, as well as CAT scan and MRI probe. This device for detecting mutagenic changes in a tissue, said device comprising: a polarizing light probe for subjecting a tissue sample to polarizing light and obtaining the associated birefringence luminescence signals; a polarizing fiber optic cable operably connected to the polarizing light probe; a digital display device operably connected to the polarizing fiber optic cable for observation of the birefringence light signals. The device wherein the polarizing light probe and endoscopy avoids transmission of heat to examine living tissue at body temperature 37 degrees centigrade.

The device further comprising software capable of analyzing the birefringence light signals transmitted to the digital display device.

The device wherein the polarizing light penetrates the tissue sample to a depth of least 100 microns with adequate lighting of 500-1500 Watt bulb, air-cooled, to view living tissue without surgical biopsy, to avoid eating of tissue this light is transmitted first to the fiber-optic cable which can tolerate the heat, whereas the light but not the heat is transmitted to the living tissue.

The device adaptable to clinical setting visualization requirements such as endoscopy, surgery, surgical subspecialties such as neurosurgery.

The device wherein the adaptation includes use with a dissecting microscope or inverted microscope to give adequate room for living tissue examination.

The device for use in assessing wound or disease biomarkers of health or disease tissue.

The device further comprising use to assess tissue deficiency states.

The device further comprising a use to assess tissue deficiency states and associated biomarkers.

### EXAMPLES

### Example Group 1

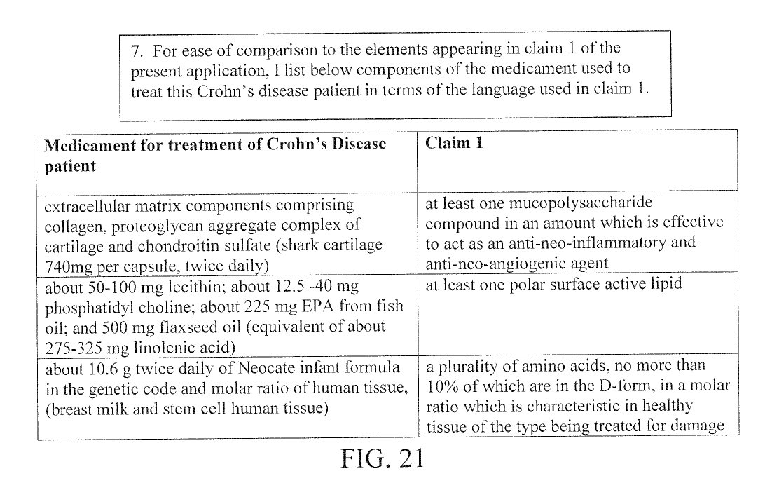

Progressive activation of the cell and stem cell tissue assembly system, inactivated by diseased tissue. The subject therapeutic use in averting the need for major organ transplantation including averting full thickness skin graft. Averting the need for joint prosthesis in activating this cell & stem cell tissue assembly system, utilizing molecular embryologic technology of organogenesis in restructuring major organ systems applied here to the stem cells found at the distal stem cell end of the bronchiolar system in asthma, the distal stem cell end of the ductules in the liver and the distal stem cell end of the kidney tubules at the site of Bowman's capsule. This includes 347 clinical responses, 60 transdermal lesional applications: 27 preclinical veterinary care, plus 33 clinical studies of resolution of arthritis sports medicine trauma in-vivo. 71 in-vitro studies of human tissue in disease resolution, reversal, transdermal lesional, oral, I.V. administration. (See FIG. 21).

Anti-oxidant co-enzyme, anti-inflammatory, multi-vitamin and minerals and trace elements have also been included (Daily Two, by Twinlab, American Fork, Utah) ideally natural beta-carotene is preferable.

A plurality of the 20 alpha amino acids (9 essential, 11 nonessential) specified by the genetic code of human and mammalian tissue have been used here in bringing about disease resolution in averting the need for surgical care for hip prosthesis (Example group 3, Case 14b). Food Carotene 1000 mg by Solaray, Park City, Utah, has been used to provide natural beta-carotene 10,000 units per soft gel. Another multi-vitamin that has been used as is from Whole Foods derived without any synthetic chemical origin and therefore has the natural form of beta-carotene and folic acid as methyl folate and the natural form as it occurs in human and plant tissue of vitamin B12 as methyl cobalamin, methyl B12, in contrast to cyanocobalamin with its potential side effects, being that it is not equivalent to human or plant tissue origin (4 capsules daily, Vitamin Code, West Palm Beach, Fla.). Omega-3 fish oil 1 g, Carlson, Arlington Heights, Ill., and alpha linolenic acid as a flax seed oil, Udo's 1-3 tablespoons 2-3 times daily.

### Example Group 2

### Equine Veterinarian

To quote the equine veterinarian, 100% efficacy was achieved with this therapeutic subject composition of matter whereas the most advanced equine veterinarian therapeutics was not effective.

Formulation ratios and amounts to provide a final transdermal delivery system lotion product with dosage for humans is about ¼ to ½ teaspoon, applied locally to the skin in the horses, the dosage is 4 to 8 times greater because of their increased six fold weight over humans and an increased surface area and tissue area for transdermal absorption, and penetrants to subjacent tissues, and disease reversal:
about 1 to 2 tablespoons Glucosamine Chondroitin MSM Cream (Nature's Plus, Melville, N.Y.)
about 1 to 2 tablespoons Vitamin A&D Zinc Oxide Cream (Schering-Plough Healthcare Products, Inc, Memphis, Tenn.)
about 1 to 2 tablespoons Vitamin E Cream (Jason Natural Products, Culver City, Calif.)—about 1 to 2 capsules Branched Chain Amino Acids—Valine, Leucine & Isoleucine (Life Extensions, Ft. Lauderdale, Fla.)
about ¼ to ½ teaspoon de-oiled Soy Lecithin Powder (Solec, St. Louis, Mo.) or 1,200 mg lecithin capsules containing 400 mg Phosphatidyl Choline (Carlson Laboratories, Arlington Heights, Ill.)
2 to 3 times daily applied Heating Pad after local application
Neocate (SHS International Ltd., Liverpool, UK) 1 teaspoon in addition to one capsule of branched chain amino acids Life Extension and Ft. Lauderdale Fla., stirred well in the foregoing well mixed formulation
about 1 to 2 tablespoons MSM Liposome Lotion (Now Foods Bloomingdale, Ill.) this has been added to further synergize the healing protein synthesis, anti-inflammatory therapeutics applied to torn rotator cuff as well as the musculoskeletal therapeutics applied to Equine veterinary medicine.

This also provides a methodology for drug discovery by co-using in the therapeutics of an existing veterinarian disease. At the same time, this represents a method of drug discovery in animal testing with use in an existing analog disease model. This was accomplished without experimentally producing a disease model in an animal, representing an ethical and humane advance in drug discovery as well as a significant economic advantage in greatly minimizing the costs that are escalating in drug development by accomplishing two beneficial therapeutic effects at the same time.

“This transdermal stem cell repair cream supplied has been very successful in over 27 cases of tendosynovitis and joint capsular synovitis of the Equine Species. Many of these cases were refractory to other therapies including steroids, non-steroidals, shock waves, IRAP (inflammatory receptor antagonist protein), and hyaluronic acid. It appears that this product helps these cases get over the final phase of tissue repair in tissue damaged so severely that the body cannot supply raw materials necessary for healing, and consequently reduce inflammation necessary for tissue homeostasis” (since updated to 31 cases).

The patent citation designed to further stimulate healing disease resolution and the patient's own stem-cell activation non-invasively regarding IPI's immunotherapeutic agent (IA) further synergized by the plant-derived adaptogen phytoalexin grape skin derived Resveratrol (muscadine grape, organically grown anti-oxidant full spectrum polyphenol flavonoids, proanthocyanidins (OPC's), anthocyanins) not only expedited healing in that the healing time response now ranged from 15 minutes to several hours (in contrast to 1-2 weeks) and “was incredible on a skin transplant that we performed on a horse. You can't even tell that he has new skin” (see FIGS. 1 and 2).

5 capsules of Wild Crafted Resveratrol 60 V-caps, 100 mg, from Genceutic Naturals, Cedar Grove, N.J., per 4 oz tube of the foregoing basic cream.

### Example Group 3

### Pain Management

Pain management alone and normalization of neuronal flow and anti-inflammatory efficacy in disease resolution with pain being one of the cardinal symptoms of inflammation which further includes heat, redness, and swelling.

Cervical Musculoskeletal Strain

These veterinary case observations and lotion therapeutic advances were further used in medical care of female, age 76, awakened from sleep with acute cervical musculoskeletal strain and arthritis. She responded after topical application of subject composition therapeutics. After treatment she was symptom free of pain with full range of cervical motion normalization upon awakening the next morning. This transdermal immunotherapeutics, anti-inflammatory, tissue healing, activation of the patient's own stem cell subject composition lotion, locally applied to the painful neck muscles 2 to 3 times daily, included:
about 1 to 2 tablespoons Glucosamine Chondroitin MSM Cream (Nature's Plus, Melville, N.Y.)
about 1 to 2 tablespoons Vitamin A&D Zinc Oxide Cream (Schering-Plough Healthcare Products, Inc, Memphis, Tenn.)
about 1 to 2 tablespoons Vitamin E Cream (Jason Natural Products, Culver City, Calif.)—about to 2 capsules Branched Chain Amino Acids Valine, Leucine & Isoleucine (Life Extensions, Ft. Lauderdale, Fla.)
about ¼ to ½ teaspoon de-oiled Soy Lecithin Powder (Solec, St. Louis, Mo.) or 1,200 mg lecithin capsules containing 400 mg Phosphatidyl Choline (Carlson Laboratories, Arlington Heights, Ill.)
2 to 3 times daily applied Heating Pad after local application
Neocate (SHS International Ltd., Liverpool, UK) 1 teaspoon in addition to one capsule of branched chain amino acids Life Extension and Ft. Lauderdale Fla., stirred well in the foregoing well mixed formulation
about 1 to 2 tablespoons MSM Liposome Lotion (Now Foods Bloomingdale, Ill.) this has been added to further synergize the healing protein synthesis, anti-inflammatory therapeutics applied to torn rotator cuff as well as the musculoskeletal therapeutics applied to Equine veterinary medicine.

Plantar Fascial Inflammation in a Female Adult Patient

This acute musculoskeletal strain and severe foot pain occurred when getting out of bed, and standing with bare feet. The strain was associated with extensive standing and walking without her customary shoe arch support. With the use of the foregoing lotion application and soak supplied for the acute gout arthritis was followed by progressive improvement over a 1 week period. The use of Advil liquid filled capsules solubilized ibuprofen locally added to the transdermal cream expedited the reversal of acute gout after one-two applications. This was administered by emptying the Advil liquid filled capsules onto the acute gout arthritis resulted in the reversal of the gout inflammation after a few applications. The orally administered treatment further included therapeutic subject composition extra-cellular matrix components chondroitin sulfate, glucosamine sulfate, MSM 2 to 3 capsules, 3 times daily; Vita Carte Bovine Cartilage, (Phoenix Biologics Vista, Calif.) 4 capsules, 3 times daily; Cartilade shark cartilage, (Ontario, Canada, formerly BioTherapies, Inc., Fairfield N.J.) 4 capsules 3 times daily, was also added to allay the severe discomfort (Now Foods Bloomingdale, Ill.); Neocate (Neocate infant formula SHS Liverpool, UK and Nutritia Gaithersburg, Md.) 1 to 2 teaspoons emulsified in 3 ounces of spring water daily; and phospholipid phosphatidyl choline 1.2 grams, 3 times daily (Carlson Laboratories, Arlington Heights, Ill.) The pain was so severe that she felt that a narcotic (such as codeine) would be necessary, however, using 2 Bufferin (GlaxoSmithKline Research Triangle Park, N.C.) functioned synergistically with complete relief of pain, obviating the need for any major pain medication.

The patient was also treated with this transdermal immunotherapeutics, anti-inflammatory, tissue healing, activation of the patient's own stem cell subject composition lotion, locally applied to the sole of the foot 2 to 3 times daily, included:
about 1 to 2 tablespoons Glucosamine Chondroitin MSM Cream (Nature's Plus, Melville, N.Y.)
about 1 to 2 tablespoons Vitamin A&D Zinc Oxide Cream (Schering-Plough Healthcare Products, Inc, Memphis, Tenn.)
about 1 to 2 tablespoons Vitamin E Cream (Jason Natural Products, Culver City, Calif.)—about 1 to 2 capsules Branched Chain Amino Acids—Valine, Leucine & Isoleucine (Life Extensions, Ft. Lauderdale, Fla.)
about ¼ to ½ teaspoon de-oiled Soy Lecithin Powder (Solec, St. Louis, Mo.) or 1,200 mg lecithin capsules containing 400 mg Phosphatidyl Choline (Carlson Laboratories, Arlington Heights, Ill.)
2 to 3 times daily applied Heating Pad after local application
Neocate (SHS International Ltd., Liverpool, UK) 1 teaspoon in addition to one capsule of branched chain amino acids Life Extension and Ft. Lauderdale Fla., stirred well in the foregoing well mixed formulation
about 1 to 2 tablespoons MSM Liposome Lotion (Now Foods Bloomingdale, Ill.) this has been added to further synergize the healing protein synthesis, anti-inflammatory therapeutics applied to torn rotator cuff as well as the musculoskeletal therapeutics applied to Equine veterinary medicine.

Example 3 dosage of transdermal cream, arthritis, tendonitis (see equine veterinarian care, rotator cuff example)

Another case report to exemplify correcting tissue deficiencies with these tissue components of subject composition therapeutic are represented by the use of our transdermal cream.

This same patient inadvertently took a larger dosage of the transdermal cream when she spilled it on her arm. She rubbed the excess spilt transdermal cream into the skin of her arm and hand, and in so doing she found that the severe tendonitis of her hand and the arthritis of her arm began to respond. In fact, it responded so dramatically that she had a 90% improvement in contrast to waiting 1 or 2 weeks for the tissue level to build up for this effect to occur. This gives us an alternate dosage in our treatment (as much as 2 ounces) to relieve a patient of immediate discomfort. Also, this shows us that the tissue level makes all the differences to achieving the reversal of this patient's tendonitis and arthritis.

Arthritis of the Hip Joint, Averting the Knee for Hip Prosthesis

Hip replacement was prescribed for a 45 year old female. She felt she was too young to have the invasive surgical care that was recommended because she was unable to walk without severe pain and limping, and required a cane. Surgery was also averted with use of subject composition therapeutics oral therapeutics. This therapeutics was administered orally 2 to 3 times daily. Her pain and range of motion normalized. This patient progressively responded to subject composition oral therapeutics, (topical therapy was not available at the time of initiation of subject composition oral therapeutics). Subject composition therapeutics was administered with some immediate response in the next 6 months. Over a period of 5 years normalization of the range of hip motion occurred and she is now able to walk 10,000 paces daily without pain, and without the need for any support.

A female patient age 45 was diagnosed in with Rheumatoid Arthritis and Osteoarthritis of the hip. Planning for hip replacement was cancelled in view of the dramatic improvement with normalization of the hip joint. Functionality of the hip joint was normalized, from a hip joint that showed no joint space and a complete arthritic fusion of the hip joint using a variant of this therapeutic subject composition.

Within a year, the patient went from being unable to walk the distance of a block without severe pain in the hip, to 5,000 paces per day. Within the next year she was able to walk 10,000 paces daily pain-free which she has maintained to date. Most recently, she was able to walk 12,000 comfortable, pain-free paces per day.

Available to the patient through component supplied by Puritan Pride, Oakdale, N.Y.

Puritan Liquid Amino capsules, code 5505 Oakdale N.Y. Detailed contents: L alanine 40 mg, L arginine 28 mg, L aspartic acid 55 mg, L. cysteine 13 mg 13 mg, L glutamine 93 mg, L. glycine 55 mg, L Histidine 8 mg, L Isoleucine 31 mg, L Leucine 31 mg, L Lysine 30 mg, L Methionine 9 mg, L phenylalanine 17 mg, L proline 28 mg, L Serine 28 mg, 1 threonine 34 mg, 1 tryptophan 6 mg, 1 valine 30 mg, L hydroxyl 26 mg, phosphatide 24 mg, phosphatide diglycerol, limositol (1-3 capsules daily). Lecithin, ultra, 1200 mg, 1 soft gel daily. Hydra Joint (vegetarian liquid glucosamine) (Cargil hydra joint glucosamine 1200 mg, and cardorotian sulfate 1200 mg, MSM (methyl sulfonyl methane) 500 mg vitamin C 300 mg per 15 cc tablespoon. Renutra, Inc. Watertown, S. Dak.), 1 half of a capful (about 1 tablespoon, 15 ml) mixed with 2 capfuls of water daily. Gelatin, Walgreen, Deerfield Ill., About 2 table spoons (1 scoop full), 1 dosage (about 2 table spoons) mixed with a half glass of fruit (orange)juice.

Vitamin E 200, 200 units, 1 soft gel daily. Evening primrose oil, 1000 mg, 1 soft gel daily. Flaxseed oil, 1000 mg, 3 soft gel daily. Garlic, 300 mg, 1 tablet daily. Ginko biloba, 100 mg, 1 to 3 soft gels daily. Hawthorne berries, 565 mg, 2 to 3 capsules daily. Vitamin K, 100 mg, 4 tablets daily. Kelp, 0.15 mg, 1 tablet daily. L-Lysine, 500 mg, 4 capsules daily. Magnesium citrate, 300 mg, 1 to 2 capsules daily. MSM, 750 mg, 3 capsules daily. Niacin, flush-free, 500 mg, 1 capsule daily. Selenium, 50 mcg, 1 capsule daily. Silymarin milk thistle, 1000 mg, 2 soft gels daily. Soylife, 750 mg, 1 to 3 capsules daily. Zinc, chelated, 50 mg, 1 tablet daily. Vitamin D 400 IU soft gel, 400 IU, 2 soft gels daily. Acidophilus capsules (milk free) containing lactobacillus blend of L. (lactobacillus) Acidophilus, L. Rhamnosus, Bifidus 2.4 billion live cells at the time of manufacture, 40 mg capsules, 1 40 mg capsule daily.

### Example Group 4a

It is believed by using these components of composition of matter immunotherapeutic agent (IA) we have normalized neuronal flow and pain management, and by normalizing the neuronal flow, even if it is in the conduction system of the heart we have therapeutically prevented not only to atrial fibrillation, but also the side effects which appear to be disturbance of normal neuronal flow.

### Example Group 4b

### Sports Medicine

Sports medicine and arthritis using the foregoing synthetic biopharmaceutical anti-inflammatory immunotherapeutic regenerative medicine disease resolution treatment in pain management activating the patient's own stem cells without the use of foreign cells and normalizing the neuronal flow, often coexistent with acute arthritis, capsular arthritis, tendonitis, tendosynovitis, and musculoskeletal damage without the use of other pain medications.

Fifty year old patient and professor wherein the addition to the transdermal cream applied to his feet and back of neuropathic pain secondary to a herniated disc from a hockey injury, wherein the pain was reversed to normal and normalization of neuronal flow was found using two principles of cell biology healing. One with regard to cobalamin that the FDA has mandated to all prenatal vitamins (methyl folate, available from Life Extension, Hollywood, Fla. 1.25 ml/ounce in a 4 ounce bottle along with a similar dose of 1.25 ml/ounce, available from Jarrow, of the methyl cobalamin). Second was the clinical observation that Alzheimer's patients had enhancement of their memory system with these two B vitamin and neurologic healing (Natural Pharmacist text book). In the transdermal cream in advancing the treatment of equine arthritis and tendonitis and tenosynovitis, we added Resveratrol (Longevinex, 1 capsule 365 mg/capsule using 1.25 capsule/ounce, also used 1200 iu of vitamin D3) to accelerate the healing of the animals and by pass the veterinarian's necessity to put the animal down.

The transdermal cream used here was the basic transdermal cream with detailed composition in Example Group 3 plus optimized folate, 1000 mcg, available by Life Extension, Ft Lauderdale, Fla., and methyl B-12 1000 mcg, available by Jarrow, Los Angeles, Calif.

Male patient, early 70s, who had cervical damage in a wrestling accident at college about age 20. Neurosurgical consultation had not been helpful. He used the foregoing transdermal dream in Example Group 2. When he recently awakened with pain in his neck, cervical pain related to his anus, and within 15-30 minutes use of the basic cream with Resveratrol modification he obtained complete relief and was able to sleep through the night without any reoccurrence of pain.

In all these example applications, inclusive of the equine veterinarian example, the transdermal cream application was preceded and used with a heating pad to augment absorption, as suggested. Minimizing the need for invasive arthroscopy (add to waiting for knee and hip).

### Example Group 5

### Gastroenterology

Applicable to inflammatory bowel disease (IBD) and is also applicable ulcerative colitis and irritable bowel syndrome (IBS). She was treated with synthetic biopharmaceutical anti-inflammatory broad-based regenerative medicine immunotherapeutics bringing about disease resolution, where we activated the patients own stem cells, as we have done in all these examples, inactivated by disease and without the use of foreign stem cells and without know undesirable effects.

Non-Surgical Wound Healing and Tissue Regeneration: Patient Scheduled for Skin Graft, Averted with IPI Therapeutics

Applied to damaged tissue, exemplified in major wound healing, averting a full thickness skin graft.

Since very little debridement was required it is evident that this inventive subject composition immunotherapeutic had a similar apoptotic effect as occurs in normal tissue

“A picture is worth 1,000 words” to be further described with the activation of apoptosis. (See FIGS. 4 and 5).

Teahon's 1991 article (discussed in the designation request) deserves special mention as it objectively highlights the therapeutic effects of elemental diet. This study examined whether treatment of acute Crohn's disease with an elemental diet improves intestinal integrity and inflammation. Teahon proposed a mechanism by which the elemental diet heals tissue, centering around the importance of the integrity of the intestinal barrier function, and reduction in inflammation. We provide below our comments to the radiographs from this article.

Upon reflecting on this case, the healing effect of F.A.S.T® on the patient's leg wound in October 2000 probably mimics the beneficial effects observed for her Crohn's disease. Her leg wound was so severely wounded as to cause her plastic surgeon to recommend a full thickness skin graft. Her prognosis was not optimal since the corticosteroid she was taking for Crohn's disease was predicted to have impaired healing both of the leg wound, and the site of skin donation. Instead of the skin graft, GIR 114P was administered both transdermally and orally and her dosage of steroids was reduced. Her leg wound healed in 4-6 weeks with minimal debridement and no sutures (steri-strip wound apposition was successfully used to close the wound). It remained healed and is free of a scar despite the magnitude of the wound (photo evidence).

### (See FIGS. 5 and 6).

FIG. 5. Pre-treatment: Intense ileitis radiograph of tagged inflammatory cells. Pretreatment abdominal scintigraph of a patient with active TBD Crohn's Disease, showing intense activity in the right iliac fossa representing ileocecal inflammation. This radiograph demonstrates the T-cells as cytotoxic and chemo-attractant effects of disease tissue associated with severe inflammation of ileitis of Crohn's disease (70% of Lymphocytes in the circulating peripheral blood stream are T-Lymphocytes, represented here radiographically as the scintillating inflammatory cells attachment, the chemical engineering bases of IBD Crohn's inflammation).

FIG. 6. Post-treatment: Repeat radiograph of ileitis cleared after 4 weeks. Repeat radiograph in same patient after 4 weeks of treatment with elemental diet. This radiograph demonstrates the therapeutic effect in providing the reversal and disease resolution by immunotherapeutics of the T-cells and their cytotoxic inflammatory affinity and the chemo-attractant effects of diseased tissue.

However the subject composition also contains additional components. To our knowledge, nobody has included in a single treatment plan all of the components described in the orphan drug designation request for the subject compositions. Our hypothesis is that the elemental diets discussed in the published literature and described in the designation request were “only” as effective as steroids because the elemental diets were not provided in the optimal molar ratio, and did not include important additional components that are necessary for cellular function and regeneration. In particular, elemental diets lack the components of the cell membrane and extracellular matrix that are provided in the subject compositions. By supplying the body with the chemical components of the extracellular matrix, such as cartilage containing chondroitin sulfate and collagen, tissue repair and anti-inflammatory activity is enhanced since the extracellular matrix provides tissues with structural support and directs cell migration, attachment, and organization. Cell membrane formation and repair is enhanced by the simultaneous administration of phospholipids, essential fatty acids, and EPA. In sum, the subject compositions are designed to repair diseased human tissue and enhance the formation of healthy tissue. Since all of the components are necessary for cellular function, all of the moieties are thought to be essential and “active.” We discussed these other moieties in the designation request, and provided references for many of the components that are not already part of the elemental diets described above. Additional references can be provided on request.

Immunopath notes that there are several examples of products that are designated as orphan drugs containing more than one “active moiety.” These include “branched chain amino acids” for the treatment of amyotrophic lateral sclerosis, Elliotts B Solution (calcium chloride; dextrose; magnesium sulfate; potassium chloride; sodium bicarbonate; sodium chloride; sodium phosphate, dibasic, heptahydrate) for use as a diluent in the intrathecal administration of methotrexate and cytarabine for the prevention or treatment meningeal leukemia and lymphocytic lymphoma; Extraneal (icodextrin 7.5% with “electrolytes”); M.V.I.-12 (ascorbic acid; biotin; cyanocobalamin; dexpanthenol; ergocalciferol; folic acid; niacinamide; pyridoxine hydrochloride; riboflavin phosphate sodium; thiamine hydrochloride; vitamin A; vitamin E) for the prevention of vitamin deficiency and thromboembolic complications in people receiving home parenteral nutrition and warfarin-type anticoagulant therapy; and Nutrineal (peritoneal dialysis solution with 1.1% “amino acids”) for use as a nutritional supplement for the treatment of malnourishment in patients undergoing continuous ambulatory peritoneal dialysis. Presumably, OOPD agreed that all of the components in these other products were “active moieties.”

When I was requested to see this patient in 2001, she had been on continuous corticosteroid treatment for Crohn's disease for thirty years. The required dosage of corticosteroid was titrated to a minimal dose in accordance with her rate of recovery from severe flare-ups, which would occur every one to three months. At that time for the flare-ups the dosage was 4-6 tablets per day for one to three days. Then in a count down fashion every two days the dosage was reduced by one tablet every 1-2 days to finally achieve the dosage of one to two tablets daily. Thus she was taking 1-6 tablets daily, and the larger dosage was dependent upon corticosteroid requirements to reverse severe flare-ups.

After the patient injured her leg, she requested a treatment that would help avert the need for a full thickness skin graft (since half the sutures were dehiscing) as well as replace the steroids that were interfering with the healing process and her recovery. Transdermal and systemic oral administration of the subject composition was used in this case and her steroid dosage was simultaneously decreased (not increased). It was found clinically that the composition not only averted the need for full thickness skin graft and the risk of creating another wound (to obtain skin for the graft, which also would have had a reduced ability to heal), but also was found to serve as a steroid substitute. About four weeks after the treatment was initiated, and at the time of the second photograph in the designation request, she had decreased her steroid consumption by about 25% (1 tablet and ½ tablet on alternating days) and now requires only 1½ A tablets weekly (½ tablet 3 times weekly).

In retrospect we can now further see another therapeutic action of the compositions of the subject invention: it progressively replaces the tissue and tissue fluid loss of 3-6 or more watery diarrheal bowel movements per day with major flare-ups and at times in between major flare-ups. Therefore, have helped not only to replace the tissue deficiencies of the wound but also for the tissue break down associated with IBD Crohn's disease.

We note that OOPD defines a pediatric population as being 16 years of age or younger.

Before the initiation of treatment, the patient was taking Aristocort 4 mg, one to two tablets daily, with higher doses necessary to treat flare-ups of Crohn's disease. She was also taking Azulfadine at a low dose.

In October 2000, the patient sustained a severe wound, out of proportion to the trauma, to her calf, as previously documented. Her physicians believed that her constant use of steroids would interfere with wound healing and wound dehiscence, and that is when I began administering GIR 114P both orally and transdermally. On the day before treatment with the subject composition was initiated in 2000, she was taking her baseline of Aristocort (one or two 4 mg tablet/day). On the day that treatment was initiated, the patient was then given a more than 25% reduction of corticosteroids: one tablet the first day and then a half of a tablet the second day, and continued in that alternate fashion. That dose of corticosteroids was gradually decreased over the next several years as therapy continued. For several years, she was taking one Aristocort tablet ever other day, which was later reduced again to one-half tablet every three days. Within the last few months, she has stopped taking corticosteroids altogether. She has continued taking a low dose of Azulfadine and using the subject composition.

Unfortunately, her primary physician has retired from practice and despite a diligent effort, I have been unable to obtain copies of his notes and records. Therefore, I am unable to provide any additional detail regarding the exact dates these reductions in Aristocort occurred. What is remarkable, however, is that despite these reductions in corticosteroid use, she has not had a single flare-up since initiating treatment with the subject composition in October 2000.

Before the initiation of therapy with the subject composition in October 2000, despite the fact that she was taking low dose of Azulfidine and Aristocort 4 mg, 1-2 tablets per day, she had flare-ups every one to three months. Flare-ups were treated by increasing her dose of corticosteroids for about four to six days. After resolution, the steroids were reduced back to about one to two tablets per day. Unfortunately, her primary physician has retired from practice and despite a diligent effort, I have been unable to obtain copies of his notes and records, or those of her gastroenterologist. Therefore, I am unable to provide any additional detail regarding the exact dates or treatment of the flare-ups.

As mentioned above, treatment with the subject composition was initiated in October 2000 and she was weaned to the most minimal dose of steroids over the next several years. Since the initiation of therapy in October 2000, she has not had a single flare-up. Therefore, there has been no referral to a gastroenterologist.

Apr. 29, 1983: Diagnosis of Crohn's disease was well established and confirmed at the Cleveland Clinic by operative and pathologic findings as well as radiological findings prior to surgery.

Oct. 28, 2000: As I have previously described, her leg was severely wounded. Although not recorded in the plastic surgeon's notes, this is when the subject compositions was administered orally and transdermally was initiated and at the same time Aristocort was progressively, but very gradually reduced.

Dec. 5, 2001: No mention of Crohn's disease.

Nov. 22, 2005: Physician's note indicates that Crohn's disease is under control, and patient takes Azulfidine.

2007-2010: Laboratory reports showing inactivity of Crohn's disease (normal sedimentation rate, normal C-reactive protein, normal CBC, and normal chronical index of Crohn's).

Aug. 31, 2010: She reports no symptoms of Crohn's disease and is not taking any medications for the treatment of Crohn's disease.

In conclusion, prior to October 2000, despite being treated with corticosteroids, this patient had several flare-ups every year. Flare-ups were treated by temporarily increasing the dose of steroids. The subject composition therapy was begun in October 2000, and corticosteroids were gradually reduced over the next several years to the most minimal dose. This patient has not taken steroids at all for the last few months. The patient has not had a single flare-up in the last ten years.

Upon reflecting on this case, the healing effect of the subject composition on the patient's leg wound in October 2000 probably mimics the beneficial effects observed for her Crohn's disease. Her leg wound was so severe as to cause her plastic surgeon to recommend a full thickness skin graft. Even then, her prognosis was not optimal since the corticosteroid she was taking for Crohn's disease were predicted to have impaired healing both of the leg wound, and the site of skin donation. Instead of the skin graft, GIR 114P was administered both transdermally and orally and her dosage of steroids was reduced. Her leg wound healed in 4-6 weeks with minimal debridement and no sutures (steri-strip wound apposition was successfully used to close the wound). It remained healed and is free of a scar despite the magnitude of the wound (photo evidence on page 11 of original application of Office of Orphan Products Development). Likewise, her gastrointestinal tract probably healed in a similar fashion. The Teahon article provided earlier provides strong support for this conclusion. Just as steroids would have interfered with her leg injury, they likely interfered with the healing of the wounds in her intestinal tract. When considering the case of pediatric Crohn's disease, the reduction (or elimination) of steroids is particularly important because of their pronounced and well-documented effects on growth and pubertal development.

Moreover, GIR 114P provided this patient with all the elements necessary to heal the wounds and regenerate the tissue in both anatomic locations. Although objective evidence of remission can not be provided for this patient, Dr. Teahon objectively showed that an elemental diet reduces intestinal permeability and inflammation. She concluded that the mechanism by which an elemental diet is effective in patients with Crohn's disease is centered around “the importance of the integrity of the intestinal barrier function.” Importantly, as mentioned throughout this letter, this patient has not had a single flare-up of Crohn's disease in the last 10 years, despite a gradual reduction and eventual elimination of all corticosteroids. I am unaware of any case showing such a profound and sustained response of treatment without known and undesirable effects.

Teahon K, Smethurst P, Pearson M, Levi A J and Bjarnason I. The effect of elemental diet on intestinal permeability and inflammation in Crohn's disease. Gastroenterol. 1991; 101: 84-8.

### Example Group 6

### Autoimmune Disease

A female patient, mid 60s, afflicted with the autoimmune disease lupuserythematosis refractory to long-term corticosteroids with arthritis of both knees, had a 50% improvement of pain, swelling, and redness of the knee arthritis when the transdermal cream was introduced in knee application, also with the above systemic care inclusive of alpha amino acids in the molar ratio of human tissue and breast milk. Tabulated oral medication as in Example Group 1. Her cognitive decline was greatly improved as well as clarity of thinking with this systemic use.

The foregoing case in Example Group 5 addressing the successful treatment of Crohn's disease represents another successful management of another autoimmune disease.

### Example Group 7

The foregoing equine veterinarian care of leg disease in an animal model using valuable large animals (race and show horses) that have a value of one half million to one million dollars with successful disease resolution and used here as a preclinical study, shown in Example 1 in the Embodiments with the humanitarian advantage of averting the need to create disease as in the comparative medicine.

They were treated with synthetic biopharmaceutical anti-inflammatory broad-based regenerative medicine immunotherapeutics bringing about disease resolution, where we activated the patients own stem cells, as we have done in all these examples, inactivated by disease and without the use of foreign stem cells and without know undesirable effects, making this further amenable to veterinarian care and the translation of similar disease model as a preclinical example of management of disease such as arthritis and sports medicine. This was presented to the orthopedic meeting by the eminent equine veterinarian and their awaiting venue of this medication.

### Example Group 8

### Anti Cancer. Preclinical and Clinical Examples

End of the cancer examples reversing predisposition of radiation oncologic burns in radiation treatment of about 5 patients, everything exemplified in examples 1, 2 and 3.

Another male patient in his early 50s, having sustained complete resection of his ulcerative colitis large bowel necessitating a colostomy, achieved reversal of metastasis of bowel cancer to the lung with the foregoing therapeutics of example 1 and discontinuing these products the cancer became reactivated and mitosis recurred.

Similar findings of an 80% prostatic cancer cell kill within 24-48 hours with the subject transdermal immunotherapeutics cream have resulted. These studies concurred with our studies of chemical moiety non-skewed to the subset that approached the 99-100% results.

In treating a multiple myeloma patient in his early 80s, with therapeutics from Example Group 1 and 2 as well as all the more detailed components on the spreadsheet.

The first group is primarily for multiple myeloma, but also for any aging patient with progressive degenerative disease.

By Cardiovascular Research Ltd, Concord, Calif.: Magnesium Taurate (125 mg, 1 capsule 3 times daily).

By Carlson, Arlington Heights, Ill.: Phosphatidyl Choline (1200 mg), Vitamin C (1000 mg, 3 times daily), Salmon Oil (1000 mg), Gamma E (500 mg), Phosphatidyl Serine (100 mg, 1 daily).

By Genceutic Naturals, Cedar Grove, N.J.: Resveratrol (100 mg, 2-3 capsules 3 times daily).

By Jarrow, Los Angeles, Calif.: Methyl B12 (1000 mg), L Carnitin (250 mg), Ubiquinol CoQ10 (200 mg), B 12 methyl cobalamin (1 mgm, 4 lozenges twice daily), Pantethine (450 mg 1-3 times daily), Ubiquinol (CoQ10) (100 mg, 1-3 times daily), Bone-Up (3 twice daily, Vitamin C, 200 mg; Vitamin D3, 1000 IU; Vitamin K, 1100 mcg; MK-7, 10 mcg; Methylcobalamin, 100 mcg; Microcrystallinehydroxyapatite, 4762 mg; Magnesium, 500 mg; Zinc, 10 mg; Copper, 1 mg; Manganese, 1 mg; Potassium, 99 mg; Boron, 3 mg).

By Life Extension, Ft. Lauderdale, Fla.: Chloropyhllin and Zinc (100 mg), Methyl Folate (1000 mg), Mega Green Tea (Green Tea (Camellia sinensis) Decaffeinated Extract (leaf) [std. to 98% polyphenols by UV (710.5 mg), 45% EGCG by HPLC (326.25 mg)], 725 mg), Super Zeaxanthin (OptiLut®, LuteinPlus® and MZ® [Marigold (Tagetes erecta) extract (flower) (free lutein equivalent 10 mg)], 38 mg; Zeaxanthin & Meso-zeaxanthin blend [micronized zeaxanthin, OptiLut®, LuteinPlus® and MZ® Marigold (Tagetes erecta) extract (flower)], 3.75 mg; C3G (Cyanidin-3-glucoside) [from European black currant (Ribes nigrum) extract (fruit)], 2.2 mg 1 capsule 1-2 times daily), Triple Action Crucifercous Vegetable Extract Indole-3-carbinol, broccoli, cabbage (Broccoli (Brassica oleracea l.) Super Concentrate Extract (plants and sprouts) [std. to 4% glucosinolates (16 mg)], 400 mg; Indole-3-Carbinol (I3C), 80 mg; Watercress (Nasturtium officinale) 4:1 Extract (leaf), 50 mg; Rosemary (Rosmarinus officinalis) Extract (leaves) [std. to 20% diterpenic compounds (10 mg), providing camosic acid/carnosol], 50 mg; Cat's Claw (Uncaria tomentosa) Extract (bark), 50 mg; Apigenin, 25 mg; Cabbage (Brassica oleracea) extract (leaf), 25 mg; DIM (Di-indolyl-methane), 14 mg), Curcumin (500 mg), Bilberry Extract (100 mg), Chromium (Niacin (as flush-free inositol hexanicotinate) 30 mg; Chromium (as chromium polynicotinate), 200 mcg; Inositol (as inositol hexanicotinate), 7.5 mg).

By Nature's Plus, Amityville, N.Y.: Acerola free of Grapefruit naringin bioflavinoid (1-3 times daily).

By Now Foods, Bloomingdale, Ill.: Grape Seed (100 mg), BCAA (L-Leucine (Free-Form) 1.6 g (1,600 mg); L-Isoleucine (Free-Form) 800 mg; L-Valine (Free-Form) 800 mg).

By Oregon's Wild Harvest, Sandy, Oreg.: Saw Palmetto (only 1 daily), Ginko Biloba (1080 mg, 3 times daily), and Garlic (1575 mg).

By Quantum, Eugene, Oreg.: Zinc Echinacea (Vitamin A (from retinyl acetate), 500 IU; Zinc (from zinc gluconate), 14 mg; Proprietary Blend, 40 mg (Slippery Elm Bark, Marshmallow Root, Echinacea Purpurea Root (4:1 extract), Elderberry Fruit (10:1 extract/5% flavonoids), Larch Arabinogalactan Gum, Mullein Leaf)).

By Solgar, Leonia, N.J.: Milk Thistle (Milk Thistle (silybum marianum) Herb and Seed Powder 350 mg, Standardised Milk Thistle Extract (80% silymarin) 100 mg).

By Source Naturals, Santa Cruz, Calif.: Pycnogenol (75 mg), L-carnitine (500 mg).

By Spring Valley, Bohemia, N.Y.: Biotin (500 mg), Lycopene NAC White, B6 (50 mg),

By Twin Lab, American Fork, Utah: Mega Soy (Novasoy Soy Bean Extract (containing 40% isoflavones [Providing 80 mg of isoflavones including: 40 mg of genestein, 31 mg of daidzein and 9 mg of glycitein]) 200 Milligrams)), B1 (100 mg), Shark Cartilage (Shark Cartilage Concentrate (standardized for 24% protein and 20% mucopolysaccharides), 1000 mg).

By Vitamin Code, West Palm Beach, Fla.: Vitamin-Mineral with the advantage of avoiding synthetic beta-carotene such as retinyl palmitate and synthetic cyanocobalamin Vitamin B12.

By Vitamin Shoppe, Northbergen, N.J.: N-acetyl L-cysteine (600 mg), Glucosamine Sulfate MSM & Chondroitin Sulfate (500 mg 3 capsules 2-3 times daily), Quercetin Bromaline (Quercetin (As Dihydrate) (Dimorphandra Gardeniaria) (Seeds) 250 Mg; Bromelain (From Pineapple) (1500 Gdu/G) 125 Mg), Heart Choice beta-sitosterol (Phytosterol Concentrate Providing Approximately: (Beta Sitosterol, 300 mg, Campesterol, 163 mg, Stigmasterol, 117 mg) 650 Mg), B2 (100 mg).

Onion (with similar but less activity as Garlic), and probiotics (VSL#3, VSL Pharmaceuticals) such as lactobacillus acidoph and lactobacillus bifidus.

Because of viscous post-nasal mucous with this patient, we added Mucinex OTC (Reckitt Benckiser, 1 tablets 2-3 times daily). Hematologic degenerative diseases such as leukemia where the foregoing Vitamin K of K2 and K7 (have been used by one of the major pharmaceutical companies, see Example Group 1 subject composition) and the physiologic logic and therapeutic rationale is that leukemia is a disease of the bone marrow which is housed or nested in the bone. This is used in the leukemia patient and is contributing to disease resolution, such as a patient aged 70 whose conditions started with myeloid dysplasia.

This treatment has been used successfully in starting a pilot study with this patient in his early 80s who was diagnosed with multiple myeloma documented by blood electrophotetic serum protein studies of gammopathy and specifally with again with the Bence-Jones protein urine study of the protein, electrophotetic studies were positive for Bence-Jones protein pathenominoc of multiple myeloma. This study was prompted by review with the director of the clinical laboratory because of a persistant increase in sedimentation rate of 60 mm per hour that was otherwise unnaccountable for. The patient was treated with subject composition, cited in Example 1, along with the Puritan liquid amino comprising a plurality of alpha amino acids. A complication of the multiple myeloma was findings of mild kidney disease inclusive of a trace of albuyme in the urine and a finding that has been present for several documents of few tenths (2-3) of a mm of normal limites of creatinine.

Multiple myeloma plasmacytoma is associated in this patient with mild kidney disease associated with a trace of albumen. The addition of N-acetyl cysteine, 500 mg, Carlson, Arlington Hills, Ill., Silymarin (milk thistle) 140 mg, Solgar, Leonia, N.J., Mega Soy, 80 mg, American Fork, Utah, Triple Action Crucifercous Vegetable Extract Indole-3-carbinol, broccoli, cabbage, Life Extension, Ft Lauderdale, Fla., Grape Seed, 100 mg, Now Foods, Bloomingdale, Ill., Chlorophyllin 100 mg with Zinc 10 mg, Life Extenstion, Ft Lauderdale, Fla. Resulting in improvement as measured by absence of trace of albumen n the urine and the remarkable normalization of the blood pressure. Melatonin, a component of normal human tissue, also has documented anti-cancer activity. W can see how this components are being used in the broad based regenerative medicine in the many disease of the elderly, starting at age 60 or in some cases as early as age 50, of cancer, coronary artery disease, arthrosclerosis, and the neurologic degernative disease such as Parkinson's and Alzheimer's. This patient also received the transdermal treatment to the spine, an area afflicted by multiple myeloma, as well as the back over the kidney area, lateral to the 01 to L5 over the posterior kidney anatomic area, helpful in the reversal of the kidney disease associated with multip myeloma, plasma cytoma.

This is further accomplished by Udo's Oil (Lynden, Wash.) comprising Alpha Linolenic Acid and omega 3 fatty acid, and Sesame extract. These last 2 items were documented by MD Anderson cancer research section devoted to Multiple Myeloma and responding to nuclear factor kappa B.

### Example Group 9

### Software

Computerized software is being developed

The 245 clinical and preclinical studies, also include 71 in-vitro studies representing 44 bio-chemicals, 27% (12 of 44) dramatizing this 3D bio-chemical trend.

This trend is also computerizable and provides positive therapeutic controlled studies results, killing in excess of 88% of cancer tissue within 24 to 48 hours. Five (5) of twelve (12) of these studies demonstrated a cancer cell kill rate of 99% to 100% in the same time frame. (These findings were verified and measured spectro-photometricly as well as microscopy). This is particularly noteworthy since these results were achieved using as safe components derived from the subject synthetic cell-like micro-environment therapeutics. Similar findings of an 80% prostatic cancer cell kill within 24-48 hours with transdermal immunotherapeutics cream have resulted. These studies concurred with our studies of chemical moiety to the subset that approached the 99-100% results and the algorithm translated into computerized codes to provide the basis of the software is further based on biophysical characteristics of this anti-cancer chemical moiety are dictated by hydrophilicity and adequate water intake (6-8 glasses daily) based on the degree of hydrogen bonding of one hydrogen bond to four and the hexagonal appearance is based on the 3-D affect of this hydrogen bonding: type of aggregation liquid crystalline phase, Geometry (microscopic), macroscopic character, approximate HLB (hydrophilic/lipophilic balance) value, and the surfactant parameter.

In the case of the highly hydrophilic: micelles (small spherical aggregates, hexagonal as the water crystal of a snowflake), cone-shaped, clear solution, HLB>13, and surfactant parameter <½.

Also hydrophilic: bilayer/lamellar phase, vertical-rectangle shape, milky dispersion, HLB 8-10, surfactant parameter ½-1.

These are exemplified when highly hydrated and highly diluted to enhance hydrophilicity and hydrogen bonding and the above biophysical characteristics of the anti-cancer algorithm result in a cancer cell kill of 70-100% (40% of the chemicals and dilutions tried). This trend is also computerizable and provides positive therapeutic controlled studies results, killing in excess of 88% of cancer tissue within 24 to 48 hours. Five (5) of twelve (12) of these studies demonstrated a cancer cell kill rate of 99% to 100% in the same time frame. (These findings were verified and measured spectro-photometricly as well as microscopy).

This is in sharp contrast to the lipophilic: reversed hexagonal phase (rods of water surrounded by emulsifier, reverse cone-shaped with a broad base, lumps of emulsifier (characteristic of the formation of tumors), HLB<6, and surfactant parameter >1. This is shown to be pro-cancer with a 50% increase in cancer cells when this surfactant cells, unfavorable to the cancer patient exemplified by PGPR with an HLB of 2 in our tabular statistics. (See FIG. 13).The World Pharmaceutical Congress presentation made by a major pharmaceutical company includes the finding of 1 of 3,000,000 computer generated chemical studies, by comparative computer analysis have been found to have anti-cancer activity (without the specification of being free of undesirable side effects). Therefore we have not only increased the probability of detecting and documenting anti-cancer activity by 104 fold but by an even greater probability factor, if one includes the freedom of undesirable effects. We have documented this efficacy in our 71 in-vitro studies, and in clinical pilot studies have demonstrated the same efficacy and safety.

### Example Group 10

### Neurologic Disease

### Example Group 11

### Dental

In a pilot study utilizing this regenerative stem cell healing system that activates the patient's own stem cells and healing system, noninvasively, without using foreign cells, this regenerative medicine immunotherapeutic anti-inflammatory agent was successful in averting patients' need for periodontal surgical care.

This eminent dentist with whom we have collaborated has practiced extensively in Michigan as well as Florida. This consultant dentist also dramatically benefited from the subject Immunotherapeutic Agent (IA) treatment for his arthritis and ligamentous tears, bringing about disease resolution and averting the need for a knee prosthesis. Thereby, he was able to remain very active, not only in his dental practice, but also in his favorite sport, golf. He felt this non-invasive anti-arthritic care would also dramatically augment his practice devoted to dental care with healing impairment such as temporo-mandibular joint arthritis. This therapeutic response and disease resolution has been repeatedly described by treating physicians and equine veterinarians for large animal with documentation as “an incredible healing response”.

This dentist examining a patient in his early 80s found that the sulcus of the right lower posterior molar bridge area was 6 mm, and by using the ubiquinol (Jarrow) and the Vitamin E 400 units (Carlson, E jems) capsules expressed and rubbed directly onto the affected gum area, and the transdermal cream with Resveratrol was used externally, with a follow-up one month later.

A second patient, in her mid 30s, had pain all the time and the tooth and gum area (the gingival area) was bleeding. We used the same treatment as in Example Group 2 over the same period of time and repeated it, and the patient has been well over the last month and a half follow-up.

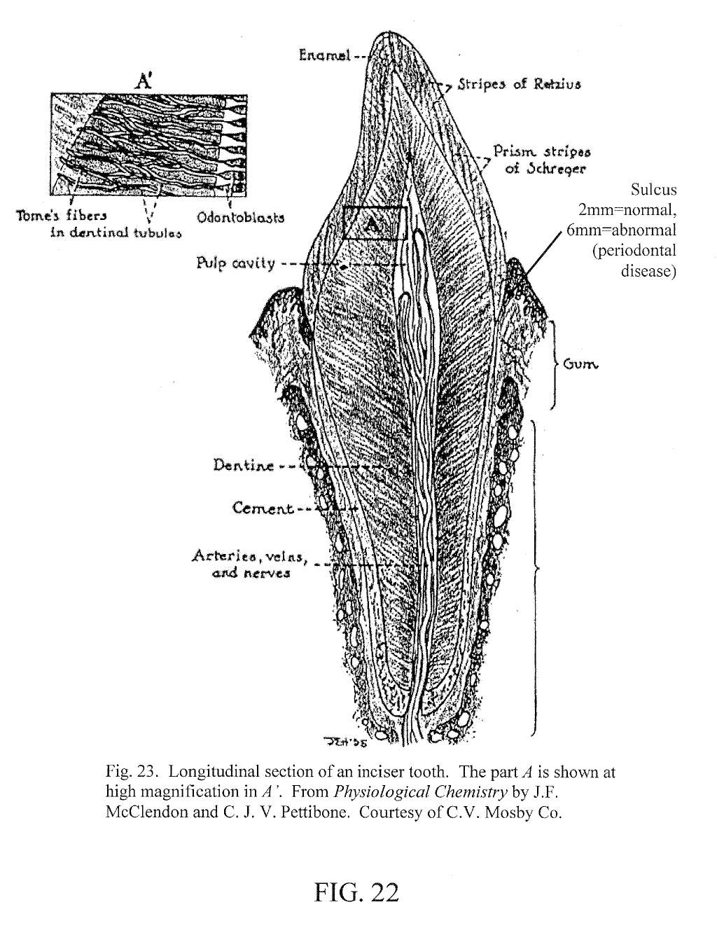

The Resveratrol cream was used with patients with temporo-mandibular joint arthritis, applied to that area externally, and the patients is showing recovery similar to the significant recovery and incredible healing seen in the treating dentists knee. (See FIG. 22).

### Example Group 12

### Wound Care & Healing

The subject Wound Healing Therapeutics was first used in the surgical care and administered intravenously such elemental feeding included 250 CC of Amigen by Baxter, operatively and post-operatively, in several hundred patients: by activating the patient's own stem cells noninvasively without the use of foreign cells we find acceleration of wound healing and post-operative recovery wherein the B service patients were able to be discharged a few days earlier than the A service surgical patients serving as a comparative control since in the case of a service the benefits of this therapeutics were not included.

This therapeutic application was best illustrated in the first child who benefited by the heart lung machine technology in pioneering as a team the first heart lung machine used in a child open-heart cardiac surgery, having been used successfully in one adult patient. This amino acid micronutrient therapeutics initiated pre-operatively and continued operatively and post operatively stimulated protein synthesis so essential to anabolic healing along with tissue differentiation in accelerating the operative and post-operative healing and recovery thereby hastening the discharge of this patient of otherwise poor prognosis.

As we study this wound, we realize that all the necrotic tissue on the upper half of the wound, (where the sutures and tissue dehisced) if treated surgically; would have required an extensive amount of debridement. When present, with each of the 7 to 10 day surgical review of the progress of this wound, very minimal debridement was required. The proof of concept was that this wound of this magnitude of necrosis when treated with IPI's therapeutic composition-of-matter required a very minimum of debridement. This is a significant advance in what is usually a labor intensive process in wound healing.

### Example Group 13

### Immediate Market Availability & Market Opportunity

The acceleration of healing applied here not only to demonstrate the most “incredible healing” and salvaging valuable horses from being put down as in the case of severe capsular arthritis, tendo-synovitis, and synovitis, in about 31 animals. This eminent equine veterinarian was invited to present his findings of therapeutic response to IA to the monthly Orthopedic Society meeting focused on advancing sports medicine therapeutics. Based on his presentation the orthopedic surgeons requested a continued update for a venue of IA therapeutics market availability.

The following is a quote from an eminent scientist, a director of a internationally prominent laboratory dedicated to the study of cancer and specifically to multiple myeloma, who viewed my recent scientific exhibit in Cambridge, Mass., summarizes our Maximizing and Optimizing the Efficacy and Safety of IPI's Immunotherapeutic Agent after viewing my technology in detail under CDA. He empathizes with my advancing therapeutics in cancer with a particular regard to this scientist's wife who is also suffering from cancer, specifically multiple myeloma:

“While I attended a conference in Boston organized by Cambridge Healthtech Institute, I met Dr. Leonard Girsh. Dr. Girsh has developed a revolutionary immunotherapeutic agent (IA) that promotes apoptosis of cancer cells and without known side effects can reestablish a normal cell cycle. Dr. Girsh has informed me that the IA is non-antigenic and could be used as a ‘drug’ or as a supplement* to present treatment remedies to hasten cancer cure*. Dr. Girsh successfully used the IA in 256 cases. He was able to reverse diseases with poor prognosis, e.g. averting the need for organ and skin transplantation and other major surgeries.”

This treatment has been used successfully in starting a poilot study group of a patient in his early 80s who was diagnosed with multiple myeloma documented by blood electrophotetic serum protein studies of gammopathy and specifally with again with the Bence-Jones protein urine study of the protein, electrophotetic studies were positive for Bence-Jones protein pathenominoc of multlple myeloma. This study was prompted by review with the director of the clinical laboratory because of a persistant increase in sedimentation rate of 60 mm per hour that was otherwise unnaccountable for. The patient was treated with subject composition, cited in Example 1, along with the Puritan liquid amino comprising a plurality of alpha amino acids.

The preclinical animal studies of IPI immunotherapeutics were supervised by one of the leading equine veterinarians in the United States providing humanitarian ethical methodology for documenting IPI's Immunotherapeutic Agent (IA) known as F.A.S.T.® anti-inflammatory disease resolution:

“The transdermal stem cell repair cream supplied has been very successful in over 27 cases of tendosynovitis and joint capsular synovitis of the Equine Species. Many of these cases were refractory to other therapies including steroids, non-steroidals, shock waves, IRAP (inflammatory receptor antagonist protein), and hyaluronic acid. It appears that this product helps these cases get over the final phase of tissue repair in tissue damaged so severely that the body cannot supply raw materials necessary for healing, and consequently reduce inflammation necessary for tissue homeostasis.”

This eminent equine veterinarian was invited to present his findings of therapeutic response to IA to the monthly Orthopedic Society meeting focused on advancing sports medicine therapeutics. Based on his presentation the orthopedic surgeons requested a continued update for a venue of IA therapeutics market availability.

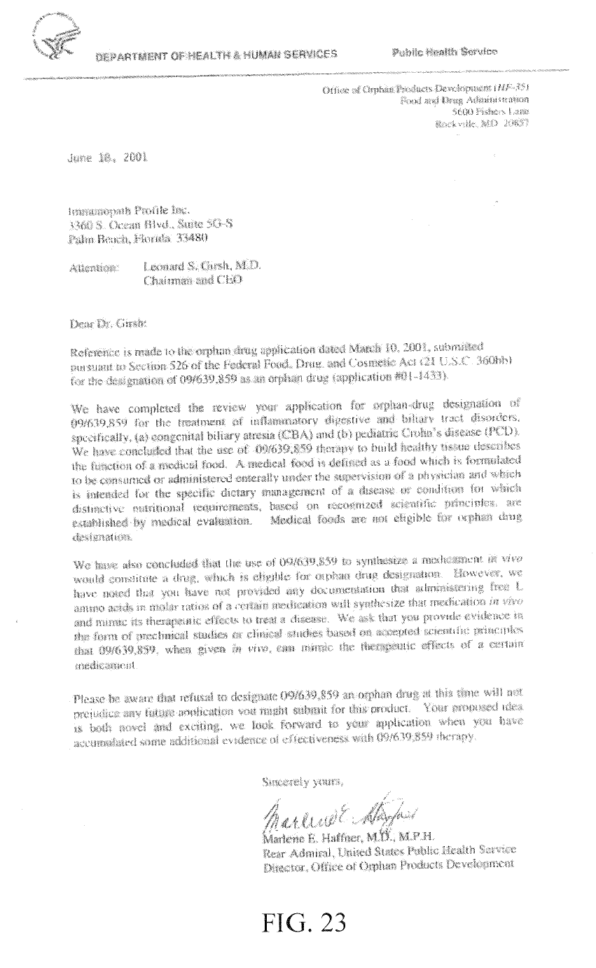

Quoting from the 2^{nd} Paragraph (See FIG. 23):

Extensively reviewed and unique to the prior art of a medical food

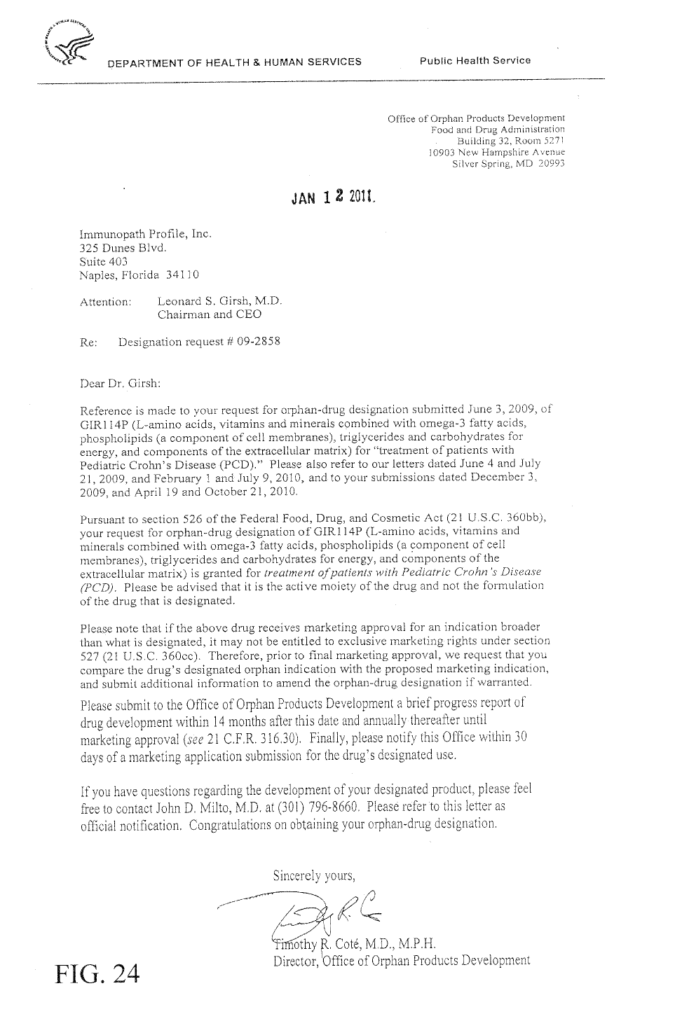

### (See FIG. 24).

### Specific Indications Averting the Need for a Liver Transplant

This synthetic biopharmaceutical immuno-therapeutic agent (IA) is also particularly effective for treating congenital biliary atresia (congenital viral hepatitis). Congenital biliary atresia (congenital viral hepatitis) is a stem cell focused disease.

This infant, age 3 weeks, weight 6 lbs. (compared to birth weight of 7 lbs.), was critically ill in a hospital with marked jaundice and abnormal liver functions. The diagnosis was congenital biliary atresia, which was substantiated surgically and the Kasai procedure was performed. A liver transplant was scheduled. A clinical strategy was considered in view of the patient's failure to thrive and the harsh planned therapy. Elemental feedings were prescribed.

Specifically, a free L amino acid powdered composition called Neocate (SHS International Ltd., U.K.) was suspended in water at a concentration of approximately 4-6 grams per 1 ounce of water, which translates into approximately 14-16 weight percent of free L amino acid, based on the total weight of the solution. This formulation was administered orally to the infant about 3-4 times a day (about 4-8 hours apart) in 6-8 ounce dosages over the period of about 6-9 months. Foods having exogenous catabolic substrate stimuli, such as all milk products, are avoided. This noninvasively activated the patient's own stem cells inactivated by disease without resorting to foreign stem cells.

A small population of stem cells located in the junction between liver cells and the smallest segments of the biliary tree may differentiate into liver cells and biliary duct epithelium and are known to participate in regeneration that occurs in certain forms of hepatitis. Thus, in congenital biliary atresia (congenital viral hepatitis) the stem cells are able to grow new tissue that are located in close proximity to the damaged tissue.

The damaged tissue has periductular inflammation and fibrosis. The locus and focus of maximal disease is at the junction of the smallest ductules of the biliary tree and the adjacent liver cells (hepatocytes), which is also the location of stem cells that are capable of regenerating liver tissue cells.

This was first applied to a child that was scheduled for a liver transplant. The child had congenital biliary atresia, but we can now look at it with more advanced technology as congenital viral hepatitis. We were able to reverse this disease by using this synthetic biopharmaceutical system, thereby averting the need for a liver transplant.

The biliary tree may be looked upon as an anatomic continuum of the small bowel. In view of this anatomic continuum the similarity of small bowel healing and biliary tract healing continuum is presented here. (See FIG. 7 showing 4 pictures: top left, a newborn with serious jaundice, needed liver transplant. Child appears to be black due to intensity of jaundice; top right: two weeks into treatment; bottom left: 3 months of treatment no longer needed liver transplant; bottom right: healthy child, positive future).

These two cases utilized synthetic biopharmaceutical anti-inflammatory broad-based regenerative medicine immunotherapeutics to directly bring about disease resolution in major joint arthritis, hip, knee, and major organ inflammatory changes.

The group of 168,000 patients in the United State waiting for an organ transplant are offered this treatment while waiting for the organ transplant and are treated with the same therapeutics. This at the same time, if any surgery is required, has enhances preoperative, peri-operative, and postoperative recovery.

This immunotherapeutic synthetic biopharmaceutical has brought about disease resolution without excising the major joint or organ, examining it pathologically, discarding it, and replacing it with an organ transplant from another patient or synthetic hip or knee prosthesis, thereby lessening significantly the morbidity and mortality and associated risks of surgical care and anesthesia as well as anti-inflammatory and immunosuppressant medications. Thereby, avoiding the concerns, in the case of major joint prosthesis, of a 10-15 re-evaluation and repeat surgical prosthesis and a similar risk if the organ transplant is rejected of subjecting the patient to similar morbidity and mortality risks and similar immunosuppressant medications to permit the successful outcome of a second organ transplant.

This broad-based regenerative medicine stem cell activation healing system based on molecular structures that underlie key cell biology systems inclusive of molecular embryology therefore the broad based efficacy that has been anticipated with the natural embryonic stem cell is being replicated as asynthetic biopharmaceutical using components equivalent to human tissue that also replace documented deficiencies in human tissue and the cell cycle by providing a composition that mimics human tissue from a biological and cell biology system and cell cycle vantage point without any known undesirable effects.

This synthetic biopharmaceutical provides the enhancement of micronutrient bioenvironment in enhancing the genomic expression to bring about significant potential reversibility to the genetic predisposition to disease. In activating the cell cycle and the proteomic display, the DNA and RNA polymerases reform to proceed with mitosis and the next cell cycle and the activation of the ribosomal templating system deactivated by diseases. This normalized cell cycle and the activation of the deactivated stem cell in disease and the sleeping egg by the hydrophilic surface-to-surface surfactant activation of enzyme substrate hydrophilicity in treating the deactivated stem cells of damaged tissue is applied to chronic persistent aggressive inflammatory disease and its complications. This efficacy provides the healing capacity and disease resolution inclusive of the emperor of all maladies, cancer, the disease that imposes continued restrictions in healing.

Genesis of Embryonic Stem Cell in Molecular Embryology:

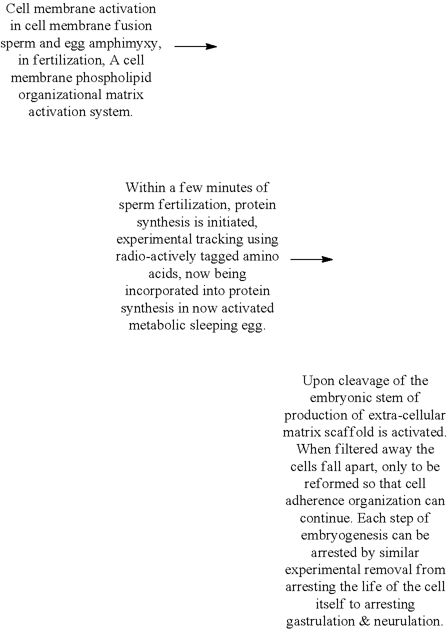

These molecular structures that underlie these key cell biology systems are very similar to activation of the sleeping egg to the cell diagram, as exemplified in activating the sleeping egg by fertilization.

The awakening of the sleeping egg by hydrophilicity can also be shown in doubling the acceleration of germination of the hydrated ribosomes, the assembly system of protein of the grass seed, by a highly diluted 1:30 of egg yolk phospholipid as in Abbott Laboratories, North Chicago, Ill., emulsified egg yolk phospholipid on the market with such names as Liposyn, preferably 1%. However we have success in cancer with 10% and 20%.

The foregoing genesis of embryonic stem cell in molecular embryology illustrates in the sleeping egg how amino acids can be in abundance, but until, it is believed, the hydrophilic activation with an HLB of 8-10 or in excess thereof, and other parameters providing a well-hydrated surfactant efficacy which in turn provides highly charged micelle repulsive particles of surface-surface enzymatic activity and the proteomic display of these foregoing essential enzymes required in templating and in the cell tissue assembly system and in the activation of apoptotic deficiency syndrome expressed in cancer and other diseases, such as the major neurologic diseases such as Parkinson's disease and Alzheimer's disease, with the inactivation of this lysosomal enzyme system, preventing the extensive accumulation of worn, damaged, and diseased neurologic tissue products requiring complete metabolism to join the basic components of IPI's immunotherapeutic agent as described. The recomposition of tissue, herein, is also dependent on abundant water intake of 6-8 glasses of water daily this highly charged micelle repulsive particle, with its surface-surface hydrophilic surfactant interaction, with particular reference to phosphatidyl choline, is important in preventing atherosclerosis and coronary artery disease, is the same surfactant in mimicking human tissue in the transdermal cream that provides its remarkable tissue permeability including the penetration of the thick hide of the horse in providing remarkable healing in arthritis and wound healing as here observed in wound healing an patient care, and as here observed by the equine veterinarian.

This report emphasizes the predisposition of disease such as cancer to deficiency of templating protein and its consequences of the fatal predisposition risk to pressure sores, decubitus ulcers, in the elderly. Best described as the “Christopher Reeve” Syndrome showing that cancer does not have to be the predisposing factor in interfering with protein templating.

Waltman N L, et al. Nutritional Status, Pressure Sores & Mortality in Elderly Patients. 1996. University of Nebraska Medical Center, College of Nursing, Omaha (17/20 patient care studies) hereby incorporated herein in its entirety by reference discloses decubitus ulcer (the “Christopher Reeves” syndrome) pressure sores related to protein deficiencies in the wound healing of decubitus ulcers in elderly nursing home patients with and without cancer predisposition. They found that 28 cancer patients had developed pressure sores versus 23 non cancer patients. 23 out of 33 patients developed Kwashiorkor (significant blood albumen protein deficiency) versus 6 out of 33 patients that had developed Kwashiorkor. In 12 weeks, 12 out of 28 (almost ½) of the cancer patients died versus the 4 out of 23 (⅙) of the non cancer patients that died.

It is believed this subject composition immunotherapeutics provides the activation of protein synthesis, an essential component of healing, associated protein polymerases of DNA and RNA, and the patient's own stem cells non-invasively. The anti-inflammatory immunotherapeutic efficacy is associated with reduction in release of inflammatory cytokines and chemo-attractants as well as an increase in the bio-chemical antagonist to interleukin inflammatory receptors associated anti-inflammatory efficacy. This subject composition therapeutics thereby provides a therapy for the elderly confined to nursing homes with the therapeutic opportunity of preventing and reversing the very serious complication of decubitus pressure sores.

This also provides a methodology for drug discovery by co-using in the therapeutics for an existing veterinarian disease such as corresponding to such as, but not limited to, diseases as treated here. At the same time this represents a method of drug discovery in animal testing with use in an existing analog disease model. This was accomplished without experimentally producing a diseased model in an animal, representing an ethical and humane advance in drug discovery as well as a significant economic advantage in greatly minimizing the costs that are escalating in drug development by accomplishing two beneficial therapeutic effects at the same time.

The 3-D stereochemistry of therapeutic compounds as amino acids without side effect and basis for stimulation of protein synthesis essential for healing and wound healing associated with alpha amino carbon and tetrahedral fit, the L amino acids and non-chiral glycine specified by the genetic code of protein and therapeutically targeted protein including anti-inflammatory efficacy by 3-D fit into protein and its synthesis including aromatic benzene ring containing amino acids such as L tyrosine L phenylalanine L tryptophan. In contrast to the 3-D sliding effect of one benzene ring upon the other as the case with aromatic benzene ring NSAID compounds is believed to explain the aromatic benzene ring containing NSAID non-steroidal anti-inflammatory drugs 3-D stereo chemical interference with protein synthesis and healing. This concurs with 20 recent studies: recommending avoidance of NSAID wound healing further evidence for this benzene ring misfit in the stereochemistry of therapeutics as applied to drug efficacy and safety and maximizing and optimizing efficacy and safety in new drug discovery.

The normalization of the mitochondria tissue energy pack from an evolutionary stand point is believed to be derived from micro-organisms. (see J. Brachet & H. Alexandre, Introduction to Molecular Embryology, Springer-Verlag Berlin Heidelberg, 1986 pg. 18, which is incorporated herein in its entirety) The normalization of the mitochondria is of practical therapeutic use as we have shown, in that with the use of phospholipid, specifically egg yolk phospholipid, normalization of the patient's energy level in end-stage cancer (available by Abbott Laboratories as Liposyn as well as a similar FDA approved product Intralipid made by Baxter Healthcare Corp., Deerfield, Ill.). This is in conjunction with existing therapy whereby patients within 24-48 hours felt that their energy level was normal and they were able to go home rather than going to hospice or remain in the hospital. This correlates with microbiologist, Warburg's studies presented at his Nobel prize presentation, in that the metabolism of mitochondria of the human cancer cells had reverted to the metabolism of the micro-organism at 10% of the aerobic energy level of normal human tissue. (Leonard S. Girsh, M.D., Lipid Containing Compositions and Methods of Using Them, US patent 2007/0037777 A1, Feb. 15, 2007 (in-vivo studies) The comparison of the more efficient aerobic energy release of human and other mammalian tissue. (see Piel, Gerard. The Age of Science. Basic Books 2001 New York, N.Y. Pages 285-286, which is incorporated herein in its entirety),) repair and tissue regeneration, which are dependent upon not only correcting and adding an adequate supply of these components, but all the components of U.S. Pat. No. 6,974,796 B1. These include amino acids, vitamins, minerals, of the elemental feedings and specific profiles of targeted tissue protein along with extracellular matrix. In fact diseases have been reported to be diagnosable by deficiency biomarker patterns. This is exemplified by neurodegenerative diseases such as MS, Parkinson's disease and Alzheimer's associated with the unique reduction of uric acid deficiency biomarker.

It has been shown here that subject opposition therapeutics has provided hydrophilic surfactant activity that contributes to reversal of diseases of poor and guarded prognosis now includes cancer supported by the associated pharmacologic mechanisms of disease reversal provided by the foregoing data included in 70 in vitro studies and associated in vivo short-term and long-term clinical studies. Recognizing the deficiencies that predisposed to diseases such as deficiencies in biamellar phospholipid such as phosphatidyl choline are included in the strategy of new drug discovery and subject composition therapeutics enhancement of disease reversal that include cancer.

Adhering to the normal HLB balance is analog to adhering in therapeutics to normal pH acid-base balance. This can be tracked clinically very simply by the use of equipment dependent upon the electrical conduction and conductance of water representing the hydrophilic component and resistance to electrical conductivity representing the lipophilic component of body tissue by the use of readily available scales such as that made by Tanita (which also measures muscle mass in addition to bone, fat, and water) or Conair Corporation that makes the scale model “Thinner” (there is also a handheld device).

The same GRAS adaptogen protecting the plant from such disease potential as bacterial infections from the soil extract from grape seed skins, Resveratrol, that synergistically when added to the transdermal cream expedited the efficacy of action from 1-2 weeks to 15 minutes to overnight. This Resveratrol (Megaresveratrol, Candlewood Stars Inc., Danbury, Conn. 250 mg, 2-4 capsules, or Wild Crafted Reseveratrol, Genceutic Naturals, Cedar Grove, N.J., 100 mg, 5 capsules per 4 ounce tube of transdermal immunotherapeutic subject composition cream, response was noted in a few days to 1-2 weeks) was added to oral administration subject composition, and because of its efficacy in normalizing the metabolic syndrome, this was added to this treatment and will be added to other patients with degenerative disease treatment synergistically optimizing and maximizing efficacy and safety with the use of this subject composition immunotherapeutics. A significant interpretation of the importance of correction of the metabolic syndrome is that lipid load inactivates the stem cell by having the stem cell convert to a preadipocyte and adipocyte. This might explain the significant problems of healing that diabetic patients have since the metabolic syndrome is also characteristic of their disease and their deficiency in healing. Also, another adaptogen that has been added is garlic (air dried Oregen Wild Harvest, Sandy, Oreg., or 1,000 mg Garlic 1 capsule, 2 to 3 times daily Nature's Bounty, Bohemia, N.Y.) organically grown with great caution in not deteriorating the active principle that is antibiotic by using air dried products ground with water cooled grinding equipment. This is to augment cardiovascular protection in that low grade septicemia is believed to play a role in intact cardiovascular endothelium such as from such hidden sources as minimal tooth decay*. This seems to be the case in the same patient in his early 80's in that he no longer has splinter hemorrhages under his finger nails as an indication of the foregoing*.

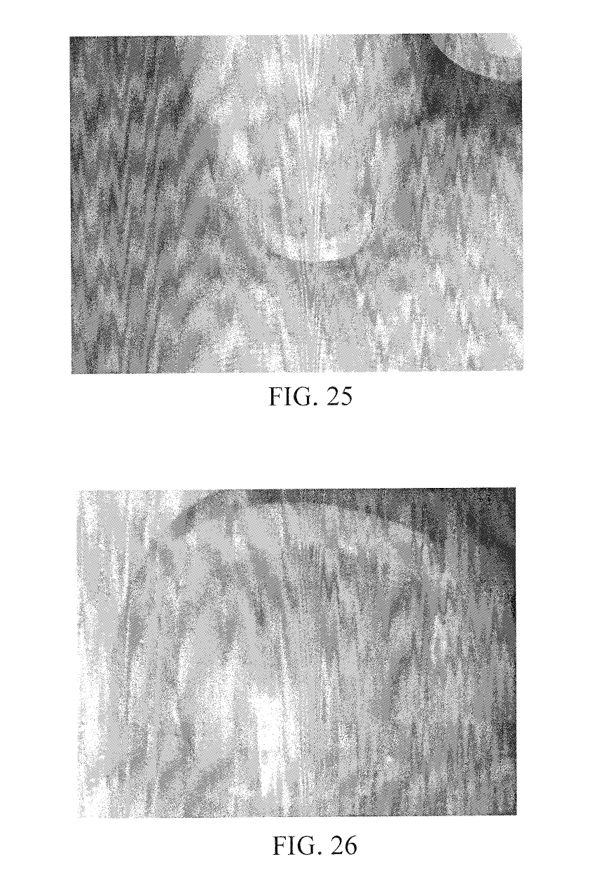

With knowledge of the field, it is easier to identify these minimal pre-monitoring findings. These examples show extreme findings. (See FIGS. 25 and 26).

### Example 1

### Treatment of Tonsillar Cancer Anal, Cancer and Small Cell Lung Cancer

At the time of treatment, Patient 1 was an approximately 35 year old male diagnosed with tonsillar cancer. His prognosis was determined to be poor. At the time of treatment, Patient 2 was an 81 year old male diagnosed with anal cancer having local dissemination. At the time of treatment, Patient 3 was an approximately 70 year old female diagnosed with intractable small cell lung cancer, with metastasis and cachexia. Patient 3 weighed approximately 90 lbs and complained of fatigue.

Patient 2 received a sterile non-pyrogenic composition of about 20% essential fatty acids (a mixture of linoleic and linolenic acid), 1.2% egg yolk phospholipid, and about 2% glycerin in water for injection. This composition is sold commercially as “Intralipid,” and was obtained from Baxter Healthcare Corp. (Deerfield, Ill.); see attached photocopy of Intralipid IV package. Intralipid is manufactured for Baxter Healthcare by Fresenius Kabi AB (Uppsala, Sweden). Five hundred milliliters of Intralipid was given intravenously three times a week for one week. Patients 1 and 3 received a similar composition called Liposyn (Abbot Laboratories) intravenously three times a week for one week. Patient 2 received 500 ml per dose and Patient 3 was administered only 250 ml per dose. Liposyn is a sterile, nonpyrogenic fat emulsion for intravenous administration, which contains about 10% safflower oil, 1.2% egg phosphatides and about 2.5% glycerin in water for injection.

Patients 1-3 showed an improvement in overall heath, including an increase in energy and relief of the fatigue and listlessness characteristic of cancer and disseminated cancer. Clinical signs of disease were visibly lessened. Regarding Patient 1, the prognosis was so grave that any signs of recovery were unexpected. However, after one week of treatment, it was reported that the improvement in Patient 1 was so dramatic that heath-care personnel had to verify the patient's name to be sure he was the same patient that had been admitted. Patient's 1 improvement continued throughout the entire course of treatment. Patient 2 was observed walking down the hall one month after treatment, and his gait was remarkably brisk in view of his age, and the extent and location of his anal cancer. Patient 3 suffered from the fatigue characteristic of cancer, particularly when complicated by metastasis and cachexia. However, 24 hours after administering the composition to Patient 3, her fatigue completely resolved, which was a completely unexpected result in a patient with such a poor prognosis. This complete clearance of fatigue was noted again, lasting for 24 hours, after administration of the next two doses.

### Example 2

Treatment of Squamous Cell Cancer, Lung Adenocarcinoma, Metastatic Lung Cancer and Anal Cancer Patient 4 is a 57-year old male with squamous cell cancer of the hypopharynx. Within one week, this patient was given a single dose of 500 ml Intralipid (20%) and a single dose of 500 ml Intralipid (10%). After this treatment, the patient showed some clinical improvement in energy levels and a decrease in lassitude.

Patient 5 is a 74-year old female with adenocarcinoma of the lung. This patient received three 500 ml doses of Intralipid (20%) in one week, and showed moderately increased energy levels and a reduction in lassitude.

Patient 6 is an 81-year old male suffering from right upper lobe lung cancer with metastatic spread in cervical lymph nodes right to left. This patient received one-daily doses of 250 ml Intralipid (10%) for 3 consecutive days. Patient 6 showed marked improvement in energy levels and a more than 60% lessening of radiation treatment side effects.

Patient 7 is an adult male, 82-years old, suffering from anal cancer complicated by severe cachexia. At the time of treatment, this patient weighed approximately 144 lbs. Upon 3 times weekly dosing with 500 ml Intralipid (20%), the size of a superficial tumor in the anaUperianal area (initially 5 cm) was reduced 40% in size after three days of treatment. After five treatments, the enlarged inguinal lymph nodes returned to normal. One indication that local dissemination of the cancer had improved.

From clinical observations of these patients before, during and after treatment with the Intralipid and Liposyn compositions, it is believed that the improvement in their clinical condition was the direct result of administering the composition.

### Example 3

### Treatment of Crohn's Disease

A 73 year old female patient suffering from Crohn's disease (symptoms included diarrhea, constipation, severe bouts of abdominal pain and fever, G.I. bleeding, generalized aching, extreme fatigue, nausea, and food and dairy intolerance) was being treated with corticosteroids administered three times weekly. This patient was administered a composition comprising about 10.6 g Neocate infant formula containing L-amino acids and glycine; about 50-100 mg lecithin; about 12.5-40 mg phosphatidyl choline; about 1000 mg fish oil concentrate (Entero-coated Fish Oil, 180 mg EPA and 120 mg DHA, Leiner Health Products, LLC, Carson Calif.); VSL (Biffidum bacterium breve, Lactobacillus acidophilus, B. bacterium longum, L. plantarum, B. bacterium infantis, L. baracaciae, Streptococcus thermophilus, L. bulgaricus) and/or Digestive Formula two-phase digestive aid available from Life Plus International, Batesville, Ak. (1 tablet daily, contains pancreatin, pancreolipase, pepsin, amylase, papain, bromelain and lipase, betaine, lactobacillus microflora such as L. salivarius, L. acidophilus, L. dds-1, L. bulgaricus and Biffidum bacteria, bile, lecithin, peppermint leaf, aloe vera and beetroot), extracellular matrix components comprising collagen, proteoglycan aggregate complex of cartilage and chondroitin sulfate (bovine and/or shark cartilage, four 740 mg capsules, twice or more daily), 3 mg boron, micocrystalline hydroxyapaptite (4762 mcg supplied as “Boneup” from Jarrow Formulas, Los Angeles, Calif., of which 1000 mcg is Ca, 510 mcg is P, and 1514 mcg is protein, 500 mg magnesium oxide, 10 mg zinc monomethionate, 1 mg copper gluconate, 1 mg manganese citrate, 300 mg glucosamine, 200 mg Vitamin C, 500 IU of Vitamin D3, 100 mcg Vitamin K as menaquinone-7, 400 mcg folic acid, and 100 mcg Vitamin B12), 200 mcg selenium, an additional 500-1000 mcg Vitamin B12, and 5 ml (preferably at bedtime) daily to three times a week of cherry flavored potassium chloride oral solution U.S.P. 10% (HUMCO, Texarkana, Tx. 75501), which contains 20 mEq (1.5 g) of potassium chloride. At times, the patient was given the potassium chloride solution as much as 5 ml three times a day as much 15 ml two to three times a day. Clinical observation showed a amelioration of severe abdominal pain and diarrhea, a decrease in fatigue, and control of osteoporosis caused by steroid use, age and previous ovarectomy. Complete normality of sedimentation rate and C-reactive proteins was also observed. A recent mild myocardial infarction has prompted the addition of three 900 mg arginine capsules in addition to the above-listed components which, along with nitroglycerin patches, has served to reduce angina. A goiter present in the patient's neck has also been maintained at a manageable size, which has precluded the need for surgical removal of the goiter. It should be noted that, at time, the VSL is not swallowed, but only used as a mouth rinse because it can aggravate the patient's diarrhea. While the present invention has been described in connection with the described ents, it is understood that other similar embodiments may be used or modifications and s made to the described embodiments for performing the same function without g therefrom. Therefore, the invention should not be limited to any single embodiment, er should be construed in breadth and scope in accordance with the recitation of the appended claims.

### Example 4

### Hypertension

Patient in 80s with symptoms of acute dizziness never noted prior, whose blood pressure readings were 160/90 and as high as 190/88 was treated with subject composition of U.S. Pat. No. 6,974,796 B1 comprising the following items, in addition to composition of Example 1, Melatonin, 1 mg was used, Nature's Bounty, Bohemia, N.Y., originally used as ⅓ of a tablet, as well as the plurality of amino acids specified by the genetic code in Puritan Liquid Amino, Oakdale, N.Y. permitting a dose reduction of Benicar (Daiichi Sankyo, Inc., Parsippany, N.J.) blocking angiotensin to 5 mg daily to 3 times weekly, concurrent with monitoring the normalization of blood pressure which has been achieved and maintained, from 20 mg tablet, Olmesartan Medoxomil-Hydrochlorothiazide 1 tablet daily:

grape seed extract capsule (3 capsule 2× per day, 300 mg, Healthy Origins, Pittsburgh, Pa.),

omega 3 fatty acid fat capsule (increased to 2 capsules 3× per day, 750 mg/l gram capsule (350 mg eicosapentaenoic acid and 250 mg docosahexaenoic acid, Carlson Laboratories, Arlington Heights, Ill.)

The blood pressure readings improved to 115-130 systolic over 64-80 diastolic (confirmed in both arms), further documented with 24 hour monitoring. The symptoms of dizziness have not recurred. With stress of blood pressure readings have occasionally increased to 140/88 which improved with Benicar (20 mg tablet, Olmesartan Medoxomil-Hydrochlorothiazide) 1 tablet daily to be used if the blood pressure failed to be controlled. (Benicar HCT, Daiichi Sankyo, Inc., Parsippany, N.J.

### Example Group 14

### Kidney

Therapeutics Applicable to Nephrotoxicity and Hepatotoxicity. In the patient in his early 80s with multiple myeloma, the addition of N-acetyl cysteine, 500 mg, Carlson, Arlington Hills, Ill., Silymarin (milk thistle) 140 mg, Solgar, Leonia, N.J. as well as the Resveratrol enriched transdermal cream of Example Group 2 applied to the back over the kidney area. These are applied daily, 2-3 times daily, with continued improvement daily. The Gilbert disease, secondary to a false Lupus reaction due to exposure to anti-fungal medication, of the liver with slight increase of total bilirubin is also improved. When used transdermally on all these examples, the permeability was increased with the subsequent use of the heating pad.

### Example Group 15

### Gout

When used transdermally on all these examples, the permeability was increased with the subsequent use of the heating pad, but in the case of the Gout, it was used at its mildest level. The patient responded with disease resolution and great relief of severe pain characteristic of Gout, swelling, and redness, on occasion to augment the relief 1-2 transdermal application dosages of indocin (about) 2.5 mg of a 50 mg capsule, Iroko Pharmaceuticals, Philadelphia, Pa.) was added to the cream in. In the treatment of Gout, the patient responded first to the Resveratrol enriched transdermal cream as in Example Group 2 locally applied used 3 times daily, with Garlic powder by Oregon Wild Harvest, specially processed, air-dried, and ground with a water-cooled grinding system to avoid generating heat, was sprinkled onto the wound as an anti-fungal agent derived from the cloves and root system of the Garlic plant and serving as a GRAS item to impart the innate immunity found in the defensin adaptogen of the plant, with good success. Also, with the improvement in pain, for able to trim the nails and therefore remove fungal tissue and its associated microtoxins with great improvement and dieses resolution, and patient is symptom free of pain of chronic gout, apparently perpetuated by the microtoxin of the fungal tineapedis.

### Example 16

### Bronchial Asthma, Respiratory Disease

In about several thousand children where elemental feeding in the form of Nutramigen made by MeadJohnson, Evansville, Ind., over a period of more than 2 decades or Neocate Infant Formula, Gettysburg, Md., without EPA and DHA were given as a substitute for milk. This was a presumptive diagnosis because the skin test was usually negative did not verify the diagnosis. The treatment effect was so dramatic representing what would progressively discovered here is the activation of the patient's own stem cells noninvasively without the use of foreign cells, with the therapeutic response occurring in a few weeks (1-3).

A respiratory disease that is important in triggering bronchitis and bronchial asthma, particularly the patients prone to bronchial asthma, such as the allergic patients, about 20 patient usages wherein Zinc Echinacea by Quantum (1 tablet in lozenge form, sucked on 3-6 times daily for a few days to a week) has been noted to have a significant effect on enhancing the immune response to upper respiratory infection and pharyngitis thereby preventing a triggering of bronchitis or bronchial asthma in predisposed patients. There is no danger of toxicity in adults or children (Barrett, B., pages 229, Percival, S. S., 2000, pages 56, Parnham, M.J., 1996, Giles, J. T., 2000). In addition to subject composition of Example Group 1 and vitamins and minerals inclusive of Vitamin C, ascorbic acid,

### Example 17

### Confirmation and Conformation of the Regulatory Affairs for Immediate Market Approval in Accordance with Letter 2001 2^{nd} Paragraph and Indications Based on Scientific Studies Further Based on Scientific Principles

Quoting from the 2^{nd} Paragraph of the Letter 2001 of Regulatory Affairs Agency (FDA):

1. Extensively reviewed and unique to the prior art of a medical food.

Conforming to the Regulatory Affairs statute of a medical food. A synthetic biopharmaceutical anti-inflammatory immunotherapeutic agent using components equivalent to human tissue that also replace documented deficiencies in human tissue and the cell cycle by providing a composition that mimics human tissue from a biological and cell biology system and cell cycle vantage point without any known undesirable effects. This Colloidal & Emulsion Technology Domain, where physics, chemistry, biology, and technology meet. In the immunotherapeutic recomposition of tissue and tissue micronutrient defect concurring with the Regulatory Affairs statute of a medical food as biomarkers to activate noninvasively the patient's own stem cells and the healing system (through these molecular structures that underlie molecular embryology) all free of known undesirable effects (preapproved as safe in the Code of Federal Regulations 21—FRC21 and GRAS Regulations). A plurality of the 20 alpha amino acids (9 essential, 11 nonessential) specified by the genetic code of human and mammalian tissue wherein no more than 10% of the amino acids are in D-form have been used here in bringing about disease resolution in averting the need for surgical care for hip prosthesis, and organ and skin transplantation.

(a) iii. Indications based on scientific studies further based on scientific principles for documenting the marketing of specific indications inclusive of a) wound healing, b) burns inclusive of oncologic radiation therapy burns, c) decubitus ulcer, as well as d) arthritis, e) tendonitis, and f) tendosynovitis and its applications and indications in g) sports medicine, and h) veterinary and i) equine veterinary medicine, d) the wound that would not heal, cancer (the emperor of all maladies) with e) cancer sub-specialty multiple myeloma, f) pain management, and g) precancerous diseases IBD, Crohn's diseases ×6 increase chance of cancer, and ulcerative colitis ×30 increase chance of cancer, g) neurologic disease: Parkinson's disease and Alzheimer's disease, h) hypertension, i) Asthma, j) gout, ocular diseases: k) macular degeneration and l) herpes zoster, dermatologic disease; m) precancerous lesions, n) keloid and hypertrophic scars, o) sarcoidosis, and p) infectious disease are also available and following this trend is expected to be in the same abundance with deficiencies of human tissue as biomarkers and therapeutic targets for stem cell healing and disease resolution.

a) BIOMARKERS TISSUE DEFICITS AND THERAPEUTIC TARGETS FOR WOUND HEALING. Wound healing nutritional deficiency 581 studies, compared with: IPI's chemical moiety component to detect biomarker tissue defects in IPI's immunotherapeutic agent (IA) directed to the treatment of these biomarker tissue defects in reestablishing tissue integrity, innate immunity, and associated adaptive immunity and disease resolution 2,145 studies of efficacy with acknowledgement of tissue deficits. 55,017 studies of efficacy of IPI's chemical moiety components (without active acknowledgement of tissue deficits) in IPI's immunotherapeutic agent (IA) directed to the treatment of these biomarker tissue defects in reestablishing tissue integrity, innate immunity, and associated adaptive immunity and disease resolution
b) BIOMARKERS TISSUE DEFICITS AND THERAPEUTIC TARGETS FOR SEVERE BURNS. Decubitus ulcer nutritional deficiency 63 studies, compared with: IPI's chemical moiety component to detect biomarker tissue defects in IPI's immunotherapeutic agent (IA) directed to the treatment of these biomarker tissue defects in reestablishing tissue integrity, innate immunity, and associated adaptive immunity and disease resolution 91 studies of efficacy with acknowledgement of tissue deficits. 2,384 studies of efficacy of IPI's chemical moiety components (without active acknowledgement of tissue deficits) in IPI's immunotherapeutic agent (IA) directed to the treatment of these biomarker tissue defects in reestablishing tissue integrity, innate immunity, and associated adaptive immunity and disease resolution.
c) BIOMARKERS TISSUE DEFICITS AND THERAPEUTIC TARGETS FOR DECUBITUS ULCER. Decubitus ulcer nutritional deficiency studies, 162 compared with: IPI's chemical moiety component to detect biomarker tissue defects in IPI's immunotherapeutic agent (IA) directed to the treatment of these biomarker tissue defects in reestablishing tissue integrity, innate immunity, and associated adaptive immunity and disease resolution 1,678 studies of efficacy with acknowledgement of tissue deficits. 1,486 studies of efficacy of IPI's chemical moiety components (without active acknowledgement of tissue deficits) in IPI's immunotherapeutic agent (IA) directed to the treatment of these biomarker tissue defects in reestablishing tissue integrity, innate immunity, and associated adaptive immunity and disease resolution.
d) BIOMARKERS TISSUE DEFICITS AND THERAPEUTIC TARGETS FOR CANCER. Cancer nutritional deficiency 6,901 studies, compared with: IPI's chemical moiety component to detect biomarker tissue defects in IPI's immunotherapeutic agent (IA) directed to the treatment of these biomarker tissue defects in reestablishing tissue integrity, innate immunity, and associated adaptive immunity and disease resolution 91,436 studies of efficacy with acknowledgement of tissue deficits. 1,224,402 studies of efficacy of IPI's chemical moiety components (without active acknowledgement of tissue deficits) in IPI's immunotherapeutic agent (IA) directed to the treatment of these biomarker tissue defects in reestablishing tissue integrity, innate immunity, and associated adaptive immunity and disease resolution. These indications are further by the genetic pattern of the general principle of healing and the enhancement of the expression of the genetic templating profile and healing pattern of the genome can be seen in the 2001 study done by the Whitehead Institute. This was confirmed by 2006 NIH study that shows a 77%* genetic conformity to wound healing pattern even with the abnormality cancer. The World Health Organization's 2010 article acknowledges palliative care as part of cancer treatment. The 1996 Japanese study also shows that the same results occurred in the patients' longevity with chemotherapy as with palliative care. This subject composition immunotherapeutics is representative of a highly specific palliative care treatment as well as a noninvasive, thereby palliative modality, of tissue examination.
e) BIOMARKERS TISSUE DEFICITS AND THERAPEUTIC TARGETS FOR MULTIPLE MYELOMA. Multiple Myeloma nutritional deficiency studies, 138 compared with: IPI's chemical moiety component to detect biomarker tissue defects in IPI's immunotherapeutic agent (IA) directed to the treatment of these biomarker tissue defects in reestablishing tissue integrity, innate immunity, and associated adaptive immunity and disease resolution 329 studies of efficacy with acknowledgement of tissue deficits. 26,906 studies of efficacy of IPI's chemical moiety components (without active acknowledgement of tissue deficits) in IPI's immunotherapeutic agent (IA) directed to the treatment of these biomarker tissue defects in reestablishing tissue integrity, innate immunity, and associated adaptive immunity and disease resolution.
f) BIOMARKERS TISSUE DEFICITS AND THERAPEUTIC TARGETS FOR PAIN MANAGEMENT. Pain management nutritional deficiency 164 studies, compared with: IPI's chemical moiety component to detect biomarker tissue defects in IPI's immunotherapeutic agent (IA) directed to the treatment of these biomarker tissue defects in reestablishing tissue integrity, innate immunity, and associated adaptive immunity and disease resolution 2,839 studies of efficacy with acknowledgement of tissue deficits. 17,873 studies of efficacy of IPI's chemical moiety components (without active acknowledgement of tissue deficits) in IPI's immunotherapeutic agent (IA) directed to the treatment of these biomarker tissue defects in reestablishing tissue integrity, innate immunity, and associated adaptive immunity and disease resolution.
g) BIOMARKERS TISSUE DEFICITS AND THERAPEUTIC TARGETS FOR IBD. Crohn's Disease nutritional deficiency studies, compared with: IPI's chemical moiety component to detect biomarker tissue defects in IPI's immunotherapeutic agent (IA) directed to the treatment of these biomarker tissue defects in reestablishing tissue integrity, innate immunity, and associated adaptive immunity and disease resolution studies of efficacy with acknowledgement of tissue deficits. Studies of efficacy of IPI's chemical moiety components (without active acknowledgement of tissue deficits) in IPI's immunotherapeutic agent (IA) directed to the treatment of these biomarker tissue defects in reestablishing tissue integrity, innate immunity, and associated adaptive immunity and disease resolution
h) BIOMARKERS TISSUE DEFICITS AND THERAPEUTIC TARGETS FOR HYPERTENSION. Hypertension nutritional deficiency studies, 1,821 compared with: IPI's chemical moiety component to detect biomarker tissue defects in IPI's immunotherapeutic agent (IA) directed to the treatment of these biomarker tissue defects in reestablishing tissue integrity, innate immunity, and associated adaptive immunity and disease resolution 5,320 studies of efficacy with acknowledgement of tissue deficits. 125,504 studies of efficacy of IPI's chemical moiety components (without active acknowledgement of tissue deficits) in IPI's immunotherapeutic agent (IA) directed to the treatment of these biomarker tissue defects in reestablishing tissue integrity, innate immunity, and associated adaptive immunity and disease resolution.

The foregoing best explains the chemical moiety (approved by the Regulatory Affairs Agency) for this conference:

However, the subject composition also contains additional components. To our knowledge, nobody has included in a single treatment plan all of the components described in the orphan drug designation request for the subject composition. The recomposition of tissue and make or repair of tissue is analogous to building and or repairing a building, adhering to the templating genetic plan as well as to the cell biology cycle of the cell biology tissue system and the activation of apoptosis as the worn, diseased and damaged tissue, wherein the architectural design of the genetic system and in accord with the cell biology system will not reconstitute the tissue without the essentials of the building blocks of the building architectural genetic plan of tissue recomposition with amino acids as “bricks”, and the essence of cell membrane phospholipid as water, and the extra cellular matrix as steel (as highly sulfated polymer Kevlar, 5 times stronger than steel) hence the analog of building a building where the material is of even greater importance than the architectural genetic plan. Our hypothesis is that the elemental diets discussed in the published literature and described in the designation request were “only” as effective as steroids because the elemental diets were not provided in the optimal molar ratio, and did not include important additional components that are necessary for cellular function and regeneration. In particular, elemental diets lack the components of the cell membrane and extracellular matrix that are provided in the subject composition. By supplying the body with the chemical components of the extracellular matrix, such as cartilage containing chondroitin sulfate and collagen, tissue repair and anti-inflammatory activity is enhanced since the extracellular matrix provides tissues with structural support and directs cell migration, attachment, and organization. Cell membrane formation and repair is enhanced by the simultaneous administration of phospholipids, essential fatty acids, and EPA. In sum, the subject composition is designed to repair diseased human tissue and enhance the formation of healthy tissue. Since all of the components are necessary for cellular function, all of the moieties in the subject composition are thought to be essential and “active.” We discussed these other moieties in the designation request, and provided references for many of the components that are not already part of the elemental diets described above. Additional references can be provided on request. And further includes the activation of apoptotic enzymatic lysosomal system dependent not only on highly hydrophilic activity and associated hydrogen bonding I augmenting surface-surface interaction of substrate and enzyme but also in the activation of templating as seen in molecular embryology in activation of the sleeping egg, where the enzyme activation in the phospholipase in the head of the sperm breaks off the arm of the phospholipid lyso phosphatidyl choline and becomes much more hydrophilically active surfactant in activating ribosomal templating as well as the proteomic display of apoptosis. The further activation of the cell cycle system by activating th mitochondrial bioenergy system in providing energy for healing as well the cell cycle assembly system in augmented here and synergized by ubiquoinol, Jarrow, QH absorb 100 mg, Los Angeles, Calif., the capsule is broken and added to the transdermal cream as well as the oral preparation. addressing cancer and unprocessed debris such as in neurologic diseases, as with Parkinson's disease, Alzheimer's disease

### (See FIG. 23).

BIOMARKERS TISSUE DEFICITS AND THERAPEUTIC TARGETS
FOR MULTIPLE MYELOMA
Multiple Myeloma nutritional deficiency studies, 138 compared with:
IPI's chemical moiety component to detect biomarker tissue defects
in IPI's immunotherapeutic agent (IA) directed to the treatment of these
biomarker tissue defects in reestablishing tissue integrity, innate
immunity, and associated adaptive immunity and disease resolution 329
studies of efficacy with acknowledgement of tissue deficits 26,906 studies
of efficacy of IPI's chemical moiety components (without active
acknowledgement of tissue deficits) in IPI's immunotherapeutic agent (IA)
directed to the treatment of these biomarker tissue defects in reestablishing
tissue integrity, innate immunity, and associated adaptive immunity and
disease resolution
Precursor to Proto-plasmic Protein

| | | |
|---|---|---|
| Amino Acids | 2416 | 21 |
| Essential | 19 | 0 |
| (histidine) | | |
| (isoleucine) | 6 | 0 |
| (leucine) | 128 | 3 |
| (lysine) | 80 | 1 |
| (methionine) | 56 | 3 |
| (phenylalanine) | 1758 | 4 |
| (threonine) | 143 | 4 |
| (tryptophan) | 25 | 2 |
| (valine) | 24 | 0 |
| Non-essential | 40 | 0 |
| (alanine) | | |
| (arginine) | 42 | 2 |
| (asparagine) | 23 | 0 |
| (aspartic acid) | 21 | 0 |
| (cysteine) | 166 | 3 |
| (glutamic acid) | 34 | 1 |
| (glutamine) | 39 | 2 |
| (glycine) | 24 | 0 |
| (proline) | 65 | 1 |
| (serine) | 180 | 4 |
| (tyrosine) | 321 | 6 |
| *Homocysteine | 4 | 4 |
| Lysine | 80 | 1 |
| n-acetyl cysteine | 27 | 1 |

Cell Memb. Essence & Configuration

| | | |
|---|---|---|
| Phosphatidylcholine | 13 | 0 |
| Omega 3 | 2 | 0 |
| Omega 6 | 2 | 0 |
| Omega 9 | 1 | 0 |

Extra Cellular Matrix (ECM)

| | | |
|---|---|---|
| Collagen | 236 | 5 |
| Cartilage | 56 | 0 |
| Glucosamine SO4 | 31 | 0 |
| Chondroitin SO4 | 11 | 0 |
| MSM | 0 | 0 |
| Hyaluronic Acid | 23 | 1 |
| Hyaluronan | 33 | 1 |
| Proteoglycan | 218 | 0 |
| Glycoprotein | 2363 | 47 |
| Glycosaminoglycans* | 107 | 4 |
| Mucopolysaccharide* | 110 | |

Micronutrients

| | | |
|---|---|---|
| Minerals | 38 | 2 |
| Vitamins* | 203 | 28 |

Adjuvants
2^{nd} Tier IPI Rx

| | | |
|---|---|---|
| Garlic | 1 | 1 |
| Resveratrol | 1 | 1 |
| Curcumin | 29 | 29 |
| Probiotics | 377 | 0 |
| Cruciferous | 2 | 2 |
| vegetables | | |
| Indole 3 | 349 | 1 |
| Albumin | 641 | 13 |
| Protein | 16039 | 121 |
| Thymus | 0 | 11 |
| Nuclear Factor | 348 | |
| kappa B | | |

BIOMARKERS TISSUE DEFICITS AND THERAPEUTIC TARGETS
FOR AUTOIMMUNE DISEASE
Autoimmune disease nutritional deficiency 550 studies, compared with:
IPI's chemical moiety component to detect biomarker tissue defects in
IPI's immunotherapeutic agent (IA) directed to the treatment of these
biomarker tissue defects in reestablishing tissue integrity, innate immunity,
and associated adaptive immunity and disease resolution 13,178 studies of
efficacy with acknowledgement of tissue deficits 253,869 studies of
efficacy of IPI's chemical moiety components (without active
acknowledgement of tissue deficits) in IPI's immunotherapeutic agent (IA)
directed to the treatment of these biomarker tissue defects in
reestablishing tissue integrity, innate immunity, and associated adaptive
immunity and disease resolution
Precursor to Proto-plasmic Protein

| | | |
|---|---|---|
| Amino Acids | 10,184 | 48 |
| Essential | 233 | 9 |
| (histidine) | | |
| (isoleucine) | 59 | 2 |
| (leucine) | 391 | 24 |
| (lysine) | 544 | 18 |
| (methionine) | 435 | 32 |
| (phenylalanine) | 554 | 25 |
| (threonine) | 411 | 44 |
| (tryptophan) | 412 | 18 |
| (valine) | 176 | 8 |
| Non-essential | 1013 | 37 |
| (alanine) | | |
| (arginine) | 1085 | 46 |
| (asparagine) | 90 | 5 |
| (aspartic acid) | 438 | 15 |
| (cysteine) | 745 | 29 |
| (glutamic acid) | 1440 | 53 |
| (glutamine) | 187 | 12 |
| (glycine) | 369 | 16 |
| (proline) | 669 | 21 |
| (serine) | 1072 | 75 |
| (tyrosine) | 2609 | 173 |
| *Homocysteine | 332 | 332 |
| Lysine | 544 | 18 |
| n-acetyl cysteine | 76 | 3 |

Cell Memb. Essence & Configuration

| | | |
|---|---|---|
| Phosphatidylcholine | 251 | 1 |
| Omega 3 | 352 | 15 |
| Omega 6 | 222 | 32 |
| Omega 9 | 77 | 3 |

Extra Cellular Matrix (ECM)

| | | |
|---|---|---|
| Collagen | 6926 | 198 |
| Cartilage | 2960 | 50 |
| Glucosamine SO4 | 186 | 5 |
| Chondroitin SO4 | 215 | 5 |
| MSM | 1 | 0 |
| Hyaluronic Acid | 420 | 1 |
| Hyaluronan | 458 | 3 |
| Proteoglycan | 718 | 20 |
| Glycoprotein | 22858 | 1099 |
| Glycosaminoglycans* | 1906 | 46 |
| Mucopolysaccharide* | 1916 | 44 |

Micronutrients

| | | |
|---|---|---|
| Minerals | 605 | 110 |
| Vitamins* | 3091 | 1541 |

Adjuvants
2^{nd} Tier IPI Rx

| | | |
|---|---|---|
| Garlic | 13 | 13 |
| Resveratrol | 19 | 19 |
| Curcumin | 34 | 34 |
| Probiotics | 55 | 55 |
| Cruciferous | 4 | 4 |
| vegetables | | |
| Indole 3 | 621 | 621 |
| Albumin | 6614 | 141 |
| Protein* | 148596 | 2222 |
| Thymus | 5834 | 5833 |
| Nuclear Factor | 1042 | |
| kappa B | | |

*IPI's immunotherapeutic molecular embryologic amino acid profile for templating the proteins necessary for activation of the stem cell

BIOMARKERS TISSUE DEFICITS AND THERAPEUTIC TARGETS
FOR CANCER
Cancer nutritional deficiency 6,901 studies, compared with:
IPI's chemical moiety component to detect biomarker tissue defects
in IPI's immunotherapeutic agent (IA) directed to the treatment of these
biomarker tissue defects in reestablishing tissue integrity, innate immunity,
and associated adaptive immunity and disease resolution 91,436 studies
of efficacy with acknowledgement of tissue deficits 1,224,402 studies of
efficacy of IPI's chemical moiety components (without active
acknowledgement of tissue deficits) in IPI's immunotherapeutic agent (IA)
directed to the treatment of these biomarker tissue defects in
reestablishing tissue integrity, innate immunity, and associated adaptive
immunity and disease resolution
Precursor to Proto-
plasmic Protein

| | | |
|---|---|---|
| Amino Acids | 57453 | 2199 |
| Essential | 2425 | 64 |
| (histidine) | | |
| (isoleucine) | 605 | 11 |
| (leucine) | 6189 | 118 |
| (lysine) | 4572 | 125 |
| (methionine) | 6506 | 372 |
| (phenylalanine) | 8944 | 142 |
| (threonine) | 15213 | 753 |
| (tryptophan) | 2095 | 93 |
| (valine) | 1415 | 18 |
| Non-essential | 5337 | 93 |
| (alanine) | | |
| (arginine) | 5183 | 215 |
| (asparagine) | 1336 | 43 |
| (aspartic | 2984 | 53 |
| acid) | | |
| (cysteine) | 10177 | 257 |
| (glutamic | 3147 | 63 |
| acid) | | |
| (glutamine) | 2514 | 81 |
| (glycine) | 3271 | 70 |
| (proline) | 3564 | 81 |
| (serine) | 23605 | 919 |
| (tyrosine) | 38844 | 775 |
| *Homocysteine | 1076 | 1076 |
| Lysine | 45272 | 125 |
| n-acetyl cysteine | 1762 | 59 |

Cell Memb. Essence
& Configuration

| | | |
|---|---|---|
| Phosphatidylcholine | 2231 | 37 |
| Omega 3 | 1499 | 55 |
| Omega 6 | 942 | 72 |
| Omega 9 | 349 | 30 |

Extra Cellular Matrix
(ECM)

| | | |
|---|---|---|
| Collagen | 15751 | 319 |
| Cartilage | 6454 | 65 |
| Glucosamine | 1691 | 20 |
| SO4 | | |
| Chondroitin | 1536 | 15 |
| SO4 | | |
| MSM | 0 | 0 |
| Hyaluronic | 1916 | 20 |
| Acid | | |
| Hyaluronan | 2258 | 24 |
| Proteoglycan | 5003 | 90 |
| Glycoprotein | 117414 | 2384 |
| Glycosaminoglycans* | 7170 | 119 |
| Mucopolysaccharide* | 7264 | 121 |

Micronutrients

| | | |
|---|---|---|
| Minerals | 9903 | 531 |
| Vitamins* | 26587 | 4610 |

Adjuvants
2^{nd} Tier IPI Rx

| | | |
|---|---|---|
| Garlic | 624 | 624 |
| Resveratrol | 1141 | 1141 |
| Curcumin | 1364 | 1364 |
| Probiotics | 380 | 377 |
| Cruciferous | 594 | 594 |
| vegetables | | |
| Indole 3 | 18583 | 155 |
| Albumin | 14791 | 295 |
| Protein | 708533 | 10091 |
| Thymus | 17055 | 17010 |
| Nuclear Factor | 8736 | |
| kappa B | | |

BIOMARKERS TISSUE DEFICITS AND THERAPEUTIC TARGETS
FOR DECUBITUS ULCER
Decubitus ulcer nutritional deficiency studies, 162 compared with:
IPI's chemical moiety component to detect biomarker tissue defects in
IPI's immunotherapeutic agent (IA) directed to the treatment of these
biomarker tissue defects in reestablishing tissue integrity, innate
immunity, and associated adaptive immunity and disease resolution
1,678 studies of efficacy with acknowledgement of tissue deficits
1,486 studies of efficacy of IPI's chemical moiety components (without
active acknowledgement of tissue deficits) in IPI's immunotherapeutic
agent (IA) directed to the treatment of these biomarker tissue defects
in reestablishing tissue integrity, innate immunity, and associated
adaptive immunity and disease resolution
Precursor to Proto-plasmic Protein

| | | |
|---|---|---|
| Amino Acids | 87 | 93 |
| Essential (histidine) | 0 | 1 |
| (isoleucine) | 0 | 0 |
| (leucine) | 4 | 4 |
| (lysine) | 4 | 0 |
| (methionine) | 2 | 3 |
| (phenylalanine) | 8 | 9 |
| (threonine) | 1 | 2 |
| (tryptophan) | 1 | 2 |
| (valine) | 3 | 4 |
| Non-essential | 16 | 16 |
| (alanine) | | |
| (arginine) | 33 | 34 |
| (asparagine) | 0 | 0 |
| (aspartic | 0 | 0 |
| acid) | | |
| (cysteine) | 5 | 5 |
| (glutamic | 3 | 4 |
| acid) | | |
| (glutamine) | 4 | 4 |
| (glycine) | 3 | 3 |
| (proline) | 6 | 6 |
| (serine) | 3 | 5 |
| (tyrosine) | 8 | 13 |
| *Homocysteine | 1 | 1 |
| Lysine | 4 | 0 |
| n-acetyl cysteine | 3 | 3 |

Cell Memb. Essence & Configuration

| | | |
|---|---|---|
| Phosphatidylcholine | 4 | 4 |
| Omega 3 | 2 | 2 |
| Omega 6 | 1 | 5 |
| Omega 9 | 0 | 1 |

Extra Cellular Matrix (ECM)

| | | |
|---|---|---|
| Collagen | 90 | 113 |
| Cartilage | 15 | 17 |
| Glucosamine SO4 | 3 | 3 |
| Chondroitin SO4 | 1 | 1 |
| MSM | 0 | 0 |
| Hyaluronic Acid | 14 | 15 |
| Hyaluronan | 14 | 0 |
| Proteoglycan | 2 | 1 |
| Glycoprotein | 62 | 75 |
| Glycosaminoglycans* | 30 | 31 |
| Mucopolysaccharide* | 32 | 33 |

Micronutrients

| | | |
|---|---|---|
| Minerals | 19 | 20 |
| Vitamins* | 91 | 96 |

Adjuvants
2^{nd} Tier IPI Rx

| | | |
|---|---|---|
| Garlic | 1 | 2 |
| Resveratrol | 0 | 0 |
| Curcumin | 0 | 0 |
| Probiotics | 2 | 3 |
| Cruciferous | 0 | 0 |
| vegetables | | |
| Indole 3 | 34 | 37 |
| Albumin | 137 | 162 |
| Protein | 731 | 843 |
| Thymus | 2 | 2 |
| Nuclear Factor kappa B | 0 | 0 |

BIOMARKERS TISSUE DEFICITS AND THERAPEUTIC TARGETS
FOR DIABETES
Diabetes nutritional deficiency studies, 3,749 compared with:
IPI's chemical moiety component to detect biomarker tissue defects in
IPI's immunotherapeutic agent (IA) directed to the treatment of these
biomarker tissue defects in reestablishing tissue integrity, innate immunity,
and associated adaptive immunity and disease resolution 9,626 studies of
efficacy with acknowledgement of tissue deficits
195,960 studies of efficacy of IPI's chemical moiety components (without
active acknowledgement of tissue deficits) in IPI's immunotherapeutic
agent (IA) directed to the treatment of these biomarker tissue defects in
reestablishing tissue integrity, innate immunity, and associated adaptive
immunity and disease resolution
Precursor to Proto-plasmic Protein

| | | |
|---|---|---|
| Amino Acids | 12752 | 820 |
| Essential | 301 | 15 |
| (histidine) | | |
| (isoleucine) | 169 | 13 |
| (leucine) | 1151 | 60 |
| (lysine) | 1397 | 41 |
| (methionine) | 665 | 52 |
| (phenylalanine) | 805 | 35 |
| (threonine) | 1619 | 96 |
| (tryptophan) | 502 | 37 |
| (valine) | 509 | 20 |
| Non-essential | 2135 | 83 |
| (alanine) | | |
| (arginine) | 4604 | 311 |
| (asparagine) | 96 | 5 |
| (aspartic | 387 | 10 |
| acid) | | |
| (cysteine) | 1079 | 57 |
| (glutamic | 1716 | 73 |
| acid) | | |
| (glutamine) | 550 | 28 |
| (glycine) | 567 | 30 |
| (proline) | 1173 | 30 |
| (serine) | 2399 | 128 |
| (tyrosine) | 3851 | 234 |
| *Homocysteine | 1266 | 1266 |
| Lysine | 1397 | 41 |
| n-acetyl cysteine | 343 | 11 |

Cell Memb. Essence & Configuration

| | | |
|---|---|---|
| Phosphatidylcholine | 531 | 20 |
| Omega 3 | 787 | 35 |
| Omega 6 | 441 | 21 |
| Omega 9 | 181 | 6 |

Extra Cellular Matrix (ECM)

| | | |
|---|---|---|
| Collagen | 3515 | 143 |
| Cartilage | 304 | 17 |
| Glucosamine | 506 | 10 |
| SO4 | | |
| Chondroitin SO4 | 140 | 2 |
| MSM | 0 | 0 |
| Hyaluronic Acid | 155 | 4 |
| Hyaluronan | 176 | 4 |
| Proteoglycan | 435 | 18 |
| Glycoprotein | 9700 | 469 |
| Glycosaminoglycans* | 1459 | 44 |
| Mucopolysaccharide* | 1467 | 44 |

Micronutrients

| | | |
|---|---|---|
| Minerals | 676 | 232 |
| Vitamins* | 6430 | 1793 |

Adjuvants
2^{nd} Tier IPI Rx

| | | |
|---|---|---|
| Garlic | 90 | 90 |
| Resveratrol | 149 | 149 |
| Curcumin | 127 | 127 |
| Probiotics | 62 | 0 |
| Cruciferous | 9 | 9 |
| vegetables | | |
| Indole 3 | 1052 | 20 |
| Albumin | 10568 | 190 |
| Protein | 114682 | 2596 |
| Thymus | 885 | 87 |
| Nuclear Factor | 963 | |
| kappa B | | |

BIOMARKERS TISSUE DEFICITS AND THERAPEUTIC TARGETS
FOR HYPERTENSION
Hypertension nutritional deficiency studies, 1,821 compared with:
IPI's chemical moiety component to detect biomarker tissue defects in
IPI's immunotherapeutic agent (IA) directed to the treatment of these
biomarker tissue defects in reestablishing tissue integrity, innate immunity,
and associated adaptive immunity and disease resolution 5,320 studies
of efficacy with acknowledgement of tissue deficits
125,504 studies of efficacy of IPI's chemical moiety components (without
active acknowledgement of tissue deficits) in IPI's immunotherapeutic
agent (IA) directed to the treatment of these biomarker tissue defects in
reestablishing tissue integrity, innate immunity, and associated adaptive
immunity and disease resolution
Precursor to Proto-plasmic Protein

| | | |
|---|---|---|
| Amino Acids | 477 | 13,529 |
| Essential | 7 | 68 |
| (histidine) | | |
| (isoleucine) | 2 | 138 |
| (leucine) | 14 | 308 |
| (lysine) | 18 | 332 |
| (methionine) | 23 | 315 |
| (phenylalanine) | 17 | 2120 |
| (threonine) | 35 | 878 |
| (tryptophan) | 16 | 198 |
| (valine) | 7 | 772 |
| Non- | 17 | 679 |
| essential (alanine) | | |
| (arginine) | 300 | 4869 |
| (asparagine) | 3 | 37 |
| (aspartic | 7 | 265 |
| acid) | | |
| (cysteine) | 30 | 424 |
| (glutamic | 13 | 336 |
| acid) | | |
| (glutamine) | 8 | 123 |
| (glycine) | 11 | 305 |
| (proline) | 61 | 4679 |
| (serine) | 53 | 1185 |
| (tyrosine) | 53 | 3393 |
| *Homocysteine | 1083 | 1091 |
| Lysine | 18 | 332 |
| n-acetyl cysteine | 4 | 181 |

Cell Memb. Essence & Configuration

| | | |
|---|---|---|
| Phosphatidylcholine | 14 | 200 |
| Omega 3 | 104 | 940 |
| Omega 6 | 57 | 647 |
| Omega 9 | 20 | 345 |

Extra Cellular Matrix (ECM)

| | | |
|---|---|---|
| Collagen | 89 | 2949 |
| Cartilage | 3 | 88 |
| Glucosamine SO4 | 2 | 50 |
| Chondroitin SO4 | 0 | 65 |
| MSM | 0 | 0 |
| Hyaluronic Acid | 0 | 148 |
| Hyaluronan | 0 | 160 |
| Proteoglycan | 2 | 112 |
| Glycoprotein | 252 | 4320 |
| Glycosaminoglycans* | 14 | 1051 |
| Mucopolysaccharide* | 14 | 1060 |

Micronutrients

| | | |
|---|---|---|
| Minerals | 98 | 439 |
| Vitamins* | 932 | 2450 |

Adjuvants
2^{nd} Tier IPI Rx

| | | |
|---|---|---|
| Garlic | 111 | 111 |
| Resveratrol | 35 | 34 |
| Curcumin | 15 | 17 |
| Probiotics | 16 | 16 |
| Cruciferous | 4 | 4 |
| vegetables | | |
| Indole 3 | 20 | 2462 |
| Albumin | 106 | 5674 |
| Protein | 1124 | 65412 |
| Thymus | 11 | 193 |
| Nuclear Factor | 388 | |
| kappa B | | |

BIOMARKERS TISSUE DEFICITS AND THERAPEUTIC TARGETS
FOR IBD
Crohn's Disease nutritional deficiency studies, compared with:
IPI's chemical moiety component to detect biomarker tissue defects in
IPI's immunotherapeutic agent (IA) directed to the treatment of these
biomarker tissue defects in reestablishing tissue integrity, innate immunity,
and associated adaptive immunity and disease resolution studies of
efficacy with acknowledgement of tissue deficits
5,334 studies of efficacy of IPI's chemical moiety components (without
active acknowledgement of tissue deficits) in IPI's immunotherapeutic
agent (IA) directed to the treatment of these biomarker tissue defects in
reestablishing tissue integrity, innate immunity, and associated adaptive
immunity and disease resolution

| | |
|---|---|
| AD | PED |

Precursor to Proto-plasmic Protein

| | | |
|---|---|---|
| Amino Acids | 344 | 8 |
| Essential (histidine) | 23 | |
| (isoleucine) | 3 | |
| (leucine) | 64 | |
| (lysine) | 16 | |
| (methionine) | 33 | |
| (phenylalanine) | 29 | |
| (threonine) | 44 | |
| (tryptophan) | 25 | |
| (valine) | 9 | |
| Non-essential (alanine) | 48 | |
| (arginine) | 53 | |
| (asparagine) | 2 | |
| (aspartic | 1 | |
| acid) | | |
| (cysteine) | 42 | |
| (glutamic | 8 | |
| acid) | | |
| (glutamine) | 54 | |
| (glycine) | 68 | |
| (proline) | 25 | |
| (serine) | 90 | |
| (tyrosine) | 86 | |
| *Homocysteine | 19 | 0 |
| Lysine | 16 | |
| n-acetyl cysteine | | |

Cell Memb. Essence & Configuration

| | | |
|---|---|---|
| Phosphatidylcholine | 14 | 0 |
| Omega 3 | 45 | 0 |
| Omega 6 | | |
| Omega 9 | | |

Extra Cellular Matrix (ECM)

| | | |
|---|---|---|
| Collagen | 237 | 2 |
| Cartilage | 21 | 0 |
| Glucosamine SO4 | 58 | 0 |
| Chondroitin SO4 | 2 | 0 |
| MSM | 0 | 0 |
| Hyaluronic Acid | 6 | 0 |
| Hyaluronan | 9 | 0 |
| Proteoglycan | 12 | 0 |
| Glycoprotein | 2170 | 19 |
| Glycosaminoglycans* | 51 | 2 |
| Mucopolysaccharide* | 50 | 2 |
| GAG* | 3 | 0 |

Micronutrients

| | | |
|---|---|---|
| Minerals | 88 | 1 |
| Vitamins* | 411 | 11 |

2^{nd} Tier IPI Rx

| | | |
|---|---|---|
| Garlic | | |
| Resveratrol | | |
| Curcumin | | |
| Probiotics | 225 | 11 |
| Enzymes | 1475 | 38 |
| Cruciferous veg. | | |
| Indole 3 | | |
| Albumin | | |
| Protein | | |
| Thymus | | |
| GRAND | 5240 | 94 |
| TOTAL | | |

Ulcerative Colitis nutritional deficiency studies, compared with:
IPI's chemical moiety component to detect biomarker tissue defects in
IPI's immunotherapeutic agent (IA) directed to the treatment of these
biomarker tissue defects in reestablishing tissue integrity, innate immunity,
and associated adaptive immunity and disease resolution studies of
efficacy with acknowledgement of tissue deficits
4,784 studies of efficacy of IPI's chemical moiety components (without
active acknowledgement of tissue deficits) in IPI's immunotherapeutic
agent (IA) directed to the treatment of these biomarker tissue defects in
reestablishing tissue integrity, innate immunity, and associated adaptive
immunity and disease resolution

| | |
|---|---|
| AD | PED |

Precursor to Proto-plasmic Protein

| | | |
|---|---|---|
| Amino Acids | 345 | 0 |
| Essential (histidine) | | |
| (isoleucine) | | |
| (leucine) | | |
| (lysine) | | |
| (methionine) | | |
| (phenylalanine) | | |
| (threonine) | | |
| (tryptophan) | | |
| (valine) | | |
| Non-essential (alanine) | | |
| (arginine) | | |
| (asparagine) | | |
| (aspartic | | |
| acid) | | |
| (cysteine) | | |
| (glutamic | | |
| acid) | | |
| (glutamine) | | |
| (glycine) | | |
| (proline) | | |
| (serine) | | |
| (tyrosine) | | |
| *Homocysteine | 24 | 2 |
| Lysine | | |
| n-acetyl cysteine | | |

Cell Memb. Essence & Configuration

| | | |
|---|---|---|
| Phosphatidylcholine | 19 | 0 |
| Omega 3 | 54 | 0 |
| Omega 6 | | |
| Omega 9 | | |

Extra Cellular Matrix (ECM)

| | | |
|---|---|---|
| Collagen | 175 | 1 |
| Cartilage | 12 | 0 |
| Glucosamine SO4 | 68 | 0 |
| Chondroitin SO4 | 3 | 0 |
| MSM | 0 | 0 |
| Hyaluronic Acid | 6 | 0 |
| Hyaluronan | 7 | 0 |
| Proteoglycan | 9 | 0 |
| Glycoprotein | 1462 | 19 |
| Glycosaminoglycans* | 107 | 3 |
| Mucopolysaccharide* | 106 | 3 |
| GAG* | 0 | 0 |

Micronutrients

| | | |
|---|---|---|
| Minerals | 52 | 1 |
| Vitamins* | 260 | 6 |

2^{nd} Tier IPI Rx

| | | |
|---|---|---|
| Garlic | | |
| Resveratrol | | |
| Curcumin | | |
| Probiotics | 276 | 7 |
| Enzymes | 1733 | 24 |
| Cruciferous veg. | | |
| Indole 3 | | |
| Albumin | | |
| Protein | | |
| Thymus | | |
| GRAND | 4718 | 66 |
| TOTAL | | |

### IPI Immunotherapeutics

This synthetic biopharmaceutical comprises and provides the profiles, pathways and systems, and associated pre-clinical and clinical data. The hydrophilic surfactant such as phospholipid phosphatidylcholine activity of one of the key molecular components has been chemically engineered to significantly enhance penetration and distribution delivery of this therapeutics whether administered systemically (i.e. intravenously or orally) or transdermally (locally). Concurrently, therapeutically active in bio-robotically-self vesiculating the essence of cell membrane.

The successful integration of extensive clinical experience to include physiological chemistry, biophysics, molecular embryology, pharmacology to IPI's bio-molecular medical synthetic biopharmaceutical replication of the Chemical Engineering of Human and other Mammalian Tissue underlying biologic processes which serves as the basis for new drug discovery. This is based on many filed patents starting with U.S. Pat. No. 6,974,796B1 and composition-of-matter chemical claims with foreign filing GIR114-PCT filed in August 2009.

10,119 studies were carried out, as a research directed collation using the five biochemical groups, 1-Amino Acids and glycine specified by the genetic code of human and mammalian tissue, phospholipid phosphatidylcholine, the essence of cell membrane, extra cellular matrix glycoprotein, and vitamins and minerals. 10,119 studies were carried out without any indication of correlation in accord with cell biology systems with foregoing molecular structures that underlie these cell biology systems; protein synthesis and molecular embryology tissue requirements.

Similar deficiency patterns and celiac disease like permeability leaks have been found in schizophrenia (6)

69 studies with regard to schizophrenia and abnormal gluten immune response similar to celiac disease More than 800 studies of micronutrient deficiency patterns similar to IBD Crohn's and ulcerative colitis in diabetes mellitus (type 2)

BIOMARKERS TISSUE DEFICITS AND THERAPEUTIC TARGETS
FOR LUPUS
Lupus nutritional deficiency 248 studies, compared with:
IPI's chemical moiety component to detect biomarker tissue defects in
IPI's immunotherapeutic agent (IA) directed to the treatment of these
biomarker tissue defects in reestablishing tissue integrity, innate immunity,
and associated adaptive immunity and disease resolution 2,461 studies
of efficacy with acknowledgement of tissue deficits
42,893 studies of efficacy of IPI's chemical moiety components (without
active acknowledgement of tissue deficits) in IPI's immunotherapeutic
agent (IA) directed to the treatment of these biomarker tissue defects in
reestablishing tissue integrity, innate immunity, and associated adaptive
immunity and disease resolution
Precursor to Proto-plasmic Protein

| | | |
|---|---|---|
| Amino Acids | 21 | 1021 |
| Essential | 2 | 34 |
| (histidine) | | |
| (isoleucine) | 0 | 6 |
| (leucine) | 5 | 66 |
| (lysine) | 2 | 98 |
| (methionine) | 3 | 59 |
| (phenylalanine) | 2 | 99 |
| (threonine) | 2 | 65 |
| (tryptophan) | 2 | 46 |
| (valine) | 1 | 21 |
| Non-essential | 4 | 100 |
| (alanine) | | |
| (arginine) | 9 | 159 |
| (asparagine) | 2 | 19 |
| (aspartic | 2 | 34 |
| acid) | | |
| (cysteine) | 4 | 70 |
| (glutamic | 0 | 37 |
| acid) | | |
| (glutamine) | 0 | 21 |
| (glycine) | 4 | 41 |
| (proline) | 7 | 122 |
| (serine) | 11 | 238 |
| (tyrosine) | 32 | 376 |
| *Homocysteine | 144 | 144 |
| Lysine | 2 | 98 |
| n-acetyl cysteine | 0 | 8 |

Cell Memb. Essence & Configuration

| | | |
|---|---|---|
| Phosphatidylcholine | 0 | 84 |
| Omega 3 | 0 | 51 |
| Omega 6 | 1 | 35 |
| Omega 9 | 0 | 9 |

Extra Cellular Matrix (ECM)

| | | |
|---|---|---|
| Collagen | 7 | 1981 |
| Cartilage | 0 | 104 |
| Glucosamine SO4 | 0 | 17 |
| Chondroitin SO4 | 0 | 32 |
| MSM | 0 | 0 |
| Hyaluronic Acid | 1 | 38 |
| Hyaluronan | 1 | 38 |
| Proteoglycan | 2 | 73 |
| Glycoprotein | 278 | 4057 |
| Glycosaminoglycans* | 41 | 508 |
| Mucopolysaccharide* | 41 | 510 |

Micronutrients

| | | |
|---|---|---|
| Minerals | 14 | 117 |
| Vitamins* | 248 | 422 |

Adjuvants
2^{nd} Tier IPI Rx

| | | |
|---|---|---|
| Garlic | 0 | 0 |
| Resveratrol | 0 | 0 |
| Curcumin | 2 | 2 |
| Probiotics | 1 | 1 |
| Cruciferous | 2 | 2 |
| vegetables | | |
| Indole 3 | 72 | 72 |
| Albumin | 27 | 905 |
| Protein | 681 | 30119 |
| Thymus | 734 | 734 |
| Nuclear Factor | 101 | |
| kappa B | | |

BIOMARKERS TISSUE DEFICITS AND THERAPEUTIC TARGETS
FOR PAIN MANAGEMENT
Pain management nutritional deficiency 164 studies, compared with:
IPI's chemical moiety component to detect biomarker tissue defects in
IPI's immunotherapeutic agent (IA) directed to the treatment of these
biomarker tissue defects in reestablishing tissue integrity, innate immunity,
and associated adaptive immunity and disease resolution 2,839 studies
of efficacy with acknowledgement of tissue deficits
17,873 studies of efficacy of IPI's chemical moiety components (without
active acknowledgement of tissue deficits) in IPI's immunotherapeutic
agent (IA) directed to the treatment of these biomarker tissue defects in
reestablishing tissue integrity, innate immunity, and associated adaptive
immunity and disease resolution
Precursor to Proto-plasmic Protein

| | | |
|---|---|---|
| Amino Acids | 1578 | 4 |
| Essential | 20 | 0 |
| (histidine) | | |
| (isoleucine) | 2 | 0 |
| (leucine) | 311 | 3 |
| (lysine) | 67 | 0 |
| (methionine) | 270 | 2 |
| (phenylalanine) | 209 | 4 |
| (threonine) | 20 | 1 |
| (tryptophan) | 133 | 2 |
| (valine) | 62 | 0 |
| Non-essential | 194 | 2 |
| (alanine) | | |
| (arginine) | 218 | 5 |
| (asparagine) | 3 | 0 |
| (aspartic | 117 | 1 |
| acid) | | |
| (cysteine) | 44 | 2 |
| (glutamic | 176 | 176 |
| acid) | | |
| (glutamine) | 70 | 70 |
| (glycine) | 165 | 165 |
| (proline) | 48 | 48 |
| (serine) | 56 | 56 |
| (tyrosine) | 201 | 201 |
| *Homocysteine | 49 | 49 |
| Lysine | 67 | 0 |
| n-acetyl cysteine | 24 | 1 |

Cell Memb. Essence & Configuration

| | | |
|---|---|---|
| Phosphatidylcholine | | |
| Omega 3 | 62 | 64 |
| Omega 6 | 34 | 59 |
| Omega 9 | 17 | 24 |

Extra Cellular Matrix (ECM)

| | | |
|---|---|---|
| Collagen | 145 | 2 |
| Cartilage | 575 | 3 |
| Glucosamine SO4 | 59 | 2 |
| Chondroitin SO4 | 40 | 0 |
| MSM | 0 | 0 |
| Hyaluronic Acid | 100 | 1 |
| Hyaluronan | 104 | 1 |
| Proteoglycan | 13 | 0 |
| Glycoprotein | 376 | 5 |
| Glycosaminoglycans* | 319 | 1 |
| Mucopolysaccharide* | 320 | 1 |

Micronutrients

| | | |
|---|---|---|
| Minerals | 84 | 2 |
| Vitamins* | 348 | 178 |

Adjuvants
2^{nd} Tier IPI Rx

| | | |
|---|---|---|
| Garlic | 42 | 42 |
| Resveratrol | 19 | 19 |
| Curcumin | 30 | 30 |
| Probiotics | 162 | 162 |
| Cruciferous | 510 | 510 |
| vegetables | | |
| Indole 3 | 788 | 788 |
| Albumin | 221 | 3 |
| Protein | 9349 | 100 |
| Thymus | 30 | 30 |
| Nuclear Factor | 22 | 22 |
| kappa B | | |

BIOMARKERS TISSUE DEFICITS AND THERAPEUTIC TARGETS
FOR WOUND HEALING
Wound healing nutritional deficiency 581 studies, compared with:
IPI's chemical moiety component to detect biomarker tissue defects in
IPI's immunotherapeutic agent (IA) directed to the treatment of these
biomarker tissue defects in reestablishing tissue integrity, innate immunity,
and associated adaptive immunity and disease resolution 2,145 studies
of efficacy with acknowledgement of tissue deficits
55,017 studies of efficacy of the subject composition(s) chemical moiety
components (without active acknowledgement of tissue deficits) in the
immunotherapeutic agent (IA) directed to the treatment of these biomarker
tissue defects in reestablishing tissue integrity, innate immunity, and
associated adaptive immunity and disease resolution
Precursor to Proto-plasmic Protein

| | | |
|---|---|---|
| Amino Acids | 2199 | 113 |
| Essential | 54 | 6 |
| (histidine) | | |
| (isoleucine) | 14 | 1 |
| (leucine) | 118 | 7 |
| (lysine) | 212 | 12 |
| (methionine) | 129 | 1 |
| (phenylalanine) | 100 | 4 |
| (threonine) | 279 | 21 |
| (tryptophan) | 29 | 5 |
| (valine) | 32 | 2 |
| Non-essential | 151 | 5 |
| (alanine) | | |
| (arginine) | 349 | 18 |
| (asparagine) | 17 | 1 |
| (aspartic acid) | 88 | 3 |
| (cysteine) | 254 | 11 |
| (glutamic | 29 | 1 |
| acid) | | |
| (glutamine) | 106 | 6 |
| (glycine) | 132 | 15 |
| (proline) | 1100 | 52 |
| (serine) | 605 | 33 |
| (tyrosine) | 684 | 19 |
| *Homocysteine | 10 | 10 |
| Lysine | 212 | 12 |
| n-acetyl cysteine | 44 | 2 |

Cell Memb. Essence & Configuration

| | | |
|---|---|---|
| Phosphatidylcholine | 26 | 3 |
| Omega 3 | 52 | 8 |
| Omega 6 | 32 | 7 |
| Omega 9 | 16 | 0 |

Extra Cellular Matrix (ECM)

| | | |
|---|---|---|
| Collagen | 8541 | 204 |
| Cartilage | 2364 | 37 |
| Glucosamine SO4 | 95 | 1 |
| Chondroitin SO4 | 367 | 3 |
| MSM | 0 | 0 |
| Hyaluronic Acid | 815 | 5 |
| Hyaluronan | 881 | 7 |
| Proteoglycan | 763 | 26 |
| Glycoprotein | 4046 | 175 |
| Glycosaminoglycans* | 1680 | 27 |
| Mucopolysaccharide* | 1694 | 26 |

Micronutrients

| | | |
|---|---|---|
| Minerals | 954 | 36 |
| Vitamins* | 853 | 230 |

Adjuvants
2^{nd} Tier IPI Rx

| | | |
|---|---|---|
| Garlic | 10 | 10 |
| Resveratrol | 15 | 15 |
| Curcumin | 51 | 48 |
| Probiotics | 14 | |
| Cruciferous | 4 | 0 |
| vegetables | | |
| Indole 3 | 381 | 4 |
| Albumin | 570 | 28 |
| Protein | 23459 | 831 |
| Thymus | 110 | 4 |
| taurine | 28 | 0 |
| carnitine | 10 | 1 |
| carnosine | 39 | 1 |
| Nuclear Factor | 200 | 62 |
| kappa B | | |

BIOMARKERS TISSUE DEFICITS AND THERAPEUTIC TARGETS
FOR SEVERE BURNS
Decubitus ulcer nutritional deficiency 63 studies, compared with:
The subject composition(s) chemical moiety component to detect
biomarker tissue defects in the immunotherapeutic agent (IA) directed
to the treatment of these biomarker tissue defects in reestablishing tissue
integrity, innate immunity, and associated adaptive immunity and disease
resolution 91 studies of efficacy with acknowledgement of tissue deficits
2,384 studies of efficacy of the subject composition(s) chemical moiety
components (without active acknowledgement of tissue deficits) in the
immunotherapeutic agent (IA) directed to the treatment of these
biomarker tissue defects in reestablishing tissue integrity, innate immunity,
and associated adaptive immunity and disease resolution
Precursor to Proto-plasmic Protein

| | | |
|---|---|---|
| Amino Acids | 156 | 2 |
| Essential | 8 | 1 |
| (histidine) | | |
| (isoleucine) | 5 | 0 |
| (leucine) | 15 | 0 |
| (lysine) | 3 | 0 |
| (methionine) | 12 | 0 |
| (phenylalanine) | 31 | 2 |
| (threonine) | 1 | 0 |
| (tryptophan) | 2 | 0 |
| (valine) | 4 | 0 |
| Non-essential | 38 | 1 |
| (alanine) | | |
| (arginine) | 22 | 1 |
| (asparagine) | 1 | 0 |
| (aspartic acid) | 1 | 0 |
| (cysteine) | 17 | 0 |
| (glutamic acid) | 2 | 0 |
| (glutamine) | 41 | 2 |
| (glycine) | 15 | 2 |
| (proline) | 15 | 0 |
| (serine) | 7 | 1 |
| (tyrosine) | 28 | 3 |
| *Homocysteine | 1 | 1 |
| Lysine | 3 | 0 |
| n-acetyl cysteine | 11 | 0 |

Cell Memb. Essence & Configuration

| | | |
|---|---|---|
| Phosphatidylcholine | 4 | 0 |
| Omega 3 | 9 | 0 |
| Omega 6 | 6 | 0 |
| Omega 9 | 3 | 0 |

Extra Cellular Matrix (ECM)

| | | |
|---|---|---|
| Collagen | 113 | 4 |
| Cartilage | 36 | 1 |
| Glucosamine SO4 | 0 | 0 |
| Chondroitin SO4 | 10 | 0 |
| MSM | 0 | 0 |
| Hyaluronic Acid | 10 | 0 |
| Hyaluronan | 10 | 0 |
| Proteoglycan | 4 | 1 |
| Glycoprotein | 230 | 11 |
| Glycosaminoglycans* | 47 | 2 |
| Mucopolysaccharide* | 47 | 2 |

Micronutrients

| | | |
|---|---|---|
| Minerals | 20 | 8 |
| Vitamins* | 54 | 15 |

Adjuvants
2^{nd} Tier IPI Rx

| | | |
|---|---|---|
| Garlic | 4 | 0 |
| Resveratrol | 1 | 1 |
| Curcumin | 1 | 1 |
| Probiotics | 2 | |
| Cruciferous | 0 | 0 |
| vegetables | | |
| Indole 3 | 19 | 19 |
| Albumin | 136 | 1 |
| Protein | 1182 | 7 |
| Thymus | 1 | 1 |
| Nuclear Factor | 1 | 1 |
| kappa B | | |

BIOMARKERS TISSUE DEFICITS AND THERAPEUTIC TARGETS
FOR CARDIAC ARRHYTHMIA
Cardiac arrhythmia nutritional deficiencies, 496 studies compared with:
The subject composition(s) chemical moiety component to detect
biomarker tissue defects in the immunotherapeutic agent (IA) directed to
the treatment of these biomarker tissue defects in reestablishing tissue
integrity, innate immunity, and associated adaptive immunity and disease
resolution 1,137 studies of efficacy with acknowledgement of tissue
deficits
29,604 studies of efficacy of the subject composition(s) chemical moiety
components (without active acknowledgement of tissue deficits) in the
immunotherapeutic agent (IA) directed to the treatment of these biomarker
tissue defects in reestablishing tissue integrity, innate immunity, and
associated adaptive immunity and disease resolution
Precursor to Proto-plasmic Protein

| | | |
|---|---|---|
| Amino Acids | 1570 | 61 |
| Essential | 27 | 2 |
| (histidine) | | |
| (isoleucine) | 25 | 0 |
| (leucine) | 84 | 1 |
| (lysine) | 42 | 2 |
| (methionine) | 49 | 2 |
| (phenylalanine) | 193 | 1 |
| (threonine) | 82 | 6 |
| (tryptophan) | 29 | 3 |
| (valine) | 64 | 0 |
| Non-essential | 202 | 2 |
| (alanine) | | |
| (arginine) | 273 | 7 |
| (asparagine) | 29 | 1 |
| (aspartic acid) | 83 | 4 |
| (cysteine) | 77 | 2 |
| (glutamic | 108 | 4 |
| acid) | | |
| (glutamine) | 22 | 1 |
| (glycine) | 108 | 2 |
| (proline) | 177 | 2 |
| (serine) | 135 | 8 |
| (tyrosine) | 287 | 4 |
| *Homocysteine | 44 | 4 |
| (secondary to B | | |
| vitamin tissue | | |
| deficit) | | |
| Lysine | 42 | 2 |
| n-acetyl cysteine | 48 | 1 |

Cell Memb. Essence & Configuration

| | | |
|---|---|---|
| Phosphatidylcholine | 22 | 0 |
| Omega 3 | 453 | 8 |
| Omega 6 | 238 | 2 |
| Omega 9 | 153 | 0 |

Extra Cellular Matrix (ECM)

| | | |
|---|---|---|
| Collagen | 319 | 7 |
| Cartilage | 29 | 1 |
| Glucosamine SO4 | 4 | 0 |
| Chondroitin SO4 | 5 | 0 |
| MSM | 0 | 0 |
| Hyaluronic Acid | 0 | 0 |
| Hyaluronan | 4 | 0 |
| Proteoglycan | 3 | 0 |
| Glycoprotein | 523 | 18 |
| Glycosaminoglycans* | 523 | 3 |
| Mucopolysaccharide* | 524 | 3 |

Micronutrients

| | | |
|---|---|---|
| Minerals | 57 | 8 |
| Vitamins* | 684 | 67 |
| Magnesium | 1446 | 292 |
| Potassium | 6332 | 235 |
| Vitamin B1 | 71 | 43 |

Adjuvants
2^{nd} Tier IPI Rx

| | | |
|---|---|---|
| Garlic | 10 | 10 |
| Resveratrol | 15 | 15 |
| Curcumin | 3 | 3 |
| Probiotics | 1 | 1 |
| Cruciferous | 1 | 1 |
| vegetables | | |
| Indole 3 | 568 | 3 |
| Albumin | 526 | 3 |
| Protein | 13719 | 212 |
| Thymus | 27 | 27 |
| taurine | 39 | 3 |
| carnitine | 113 | 36 |
| carnosine | 2 | 0 |
| Nuclear Factor | 14 | 14 |
| kappa B | | |

The subject composition (IPI) chemical moiety component efficacy without deficient tissue biomarkers providing disease resolution reconstituting tissue integrity, innate immunity and adaptive immunity

The inventor discovered this concept of tissue resistance and its structural and functional identification during first year of practice as an associate professor at Temple University. As a specialist in immunology and allergy, the inventor recognized that allergy was an ‘overactive immune mother’ acting as innate immunity in rejecting pollen, mold and food allergies such as milk, chocolate, and eggs. He felt that although the cancer cell may occur, this ‘overactive immune mother’ was also rejecting it. He found there were 15 articles presented by to document this concept (ref 1958)

In 2010 the Japanese found the tissue integrity and its associated innate immunity and document that biochemically by extending on the same observation that I have made in that the associated innate immunity can be indentified by certain biochemicals. They showed the TH2 phenomenon and TH1 in sarcoid patients. They were able to show this difference and what makes this disease so mild since allergic disease is endowed with reversibility and is also explained by this adaptive immunity. In the case of the sarcoid, it doesn't coexist with allergy, the innate immunity is very disrupted and the response triggers the development of TH1 immune responses and reduced expression of regulatory T cells also contribute to the augmented TH1 disrupted innate immune response. Conversely, allergic disease, or atopy, is characterized by the production of a specific immunoglobulin E response towards environmental or food allergens in association with combating and minimizing diseases such as cancer, which as we have said from my observations that has been continued since 1958, that there is a significantly lesser incidence of cancer in the allergic population. In my first year of practice in allergy and asthma I only encountered one patient who had cancer whereas in internal medicine I encountered one patient a week. The immune response is characterized by the production of TH2 (cytokines such as IL-4 and IL-13) in addition to the immunoglobulin E response. This is in contrast to the corrosive but apparent response protection of sarcoid making it a more serious disease and TH1 cytokines, as components of the innate immunity include gamma, IL-12, IL-18, and tumor necrosis factor alpha that appear to be essential for the granuloma formation. Recent insights into the immunopathogensis of sarcoidosis suggest that a disrupted innate immune response triggers the development of TH1 immune responses. Reduced expression of regulatory T cells also contributes to the augmented TH1 immune response with the cytokines so mentioned. This reduction in the expression of disease is also atopy in association with rheumatoid arthritis and multiple sclerosis in association with allergic disease such as house dust mites specific IgE response. This shows a specific biochemical pathogenesis trend.

The diseases provided here include: wound healing, healing of burns (inclusive of oncologic radiation burns), and decubitus ulcers, all requiring the same challenge of wound healing. Second is stem cell healing activated by IPI's immunotherapeutic agent and by following not only reestablishing tissue integrity but also innate immunity and its associated adaptive immunity, but also following molecular embryology in regard to the molecular structures that underlie key cell biology systems. Finally, we have diseases that include cancer (a wound that does not heal then goes on to metastasis) as an extension of wound healing.

BIOMARKERS TISSUE DEFICITS AND THERAPEUTIC TARGETS
FOR PARKINSONISM
Parkinsonism nutritional deficiency 134 studies, compared with:
The subject composition(s) chemical moiety component to detect
biomarker tissue defects in the composition(s) immunotherapeutic
agent (IA) directed to the treatment of these biomarker tissue defects
in reestablishing tissue integrity, innate immunity, and associated
adaptive immunity and disease resolution 587 studies of efficacy with
acknowledgement of tissue deficits
48,903 studies of efficacy of the subject composition(s) chemical moiety
components (without active acknowledgement of tissue deficits) in the
immunotherapeutic agent (IA) directed to the treatment of these biomarker
tissue defects in reestablishing tissue integrity, innate immunity, and
associated adaptive immunity and disease resolution
Precursor to Proto-plasmic Protein

| | | |
|---|---|---|
| Amino Acids | 10284 | 11 |
| Essential | 43 | 0 |
| (histidine) | | |
| (isoleucine) | 14 | 0 |
| (leucine) | 315 | 0 |
| (lysine) | 55 | 0 |
| (methionine) | 179 | 1 |
| (phenylalanine) | 8611 | 3 |
| (threonine) | 578 | 0 |
| (tryptophan) | 187 | 1 |
| (valine) | 58 | 0 |
| Non-essential | 140 | 1 |
| (alanine) | | |
| (arginine) | 114 | 0 |
| (asparagine) | 29 | 0 |
| (aspartic | 156 | 0 |
| acid) | | |
| (cysteine) | 363 | 0 |
| (glutamic | 331 | 2 |
| acid) | | |
| (glutamine) | 58 | 0 |
| (glycine) | 173 | 0 |
| (proline) | 65 | 0 |
| (serine) | 728 | 0 |
| (tyrosine) | 10690 | 10 |
| *Homocysteine | 118 | 0 |
| Lysine | 55 | 0 |
| n-acetyl cysteine | 81 | 0 |

Cell Memb. Essence & Configuration

| | | |
|---|---|---|
| Phosphatidylcholine | 22 | 0 |
| Omega 3 | 34 | 0 |
| Omega 6 | 20 | 0 |
| Omega 9 | 5 | 0 |

Extra Cellular Matrix (ECM)

| | | |
|---|---|---|
| Collagen | 26 | 1 |
| Cartilage | 11 | 0 |
| Glucosamine SO4 | 1 | 0 |
| Chondroitin SO4 | 4 | 0 |
| MSM | 0 | 0 |
| Hyaluronic Acid | 2 | 0 |
| Hyaluronan | 3 | 0 |
| Proteoglycan | 16 | 0 |
| Glycoprotein | 788 | 3 |
| Glycosaminoglycans* | 30 | 0 |
| Mucopolysaccharide* | 30 | 0 |

Micronutrients

| | | |
|---|---|---|
| Minerals | 44 | 6 |
| Vitamins* | 541 | 122 |
| Magnesium | 66 | 16 |
| Selenium | 20 | 7 |
| Vitamin C | 111 | 9 |
| Vitamin B1 | 12 | 8 |
| Potassium | 174 | 10 |
| Zinc | 108 | 12 |
| Melatonin | 130 | 13 |
| Chromium | 6 | 0 |

Adjuvants
2^{nd} Tier IPI Rx

| | | |
|---|---|---|
| Garlic | 4 | 0 |
| Resveratrol | 10 | 0 |
| Curcumin | 18 | 0 |
| Probiotics | 0 | 0 |
| Cruciferous | 0 | 0 |
| vegetables | | |
| Indole 3 | 608 | 44 |
| Albumin | 93 | 5 |
| Protein | 12540 | 301 |
| Thymus | 12 | 1 |
| Nuclear Factor | 63 | |
| kappa B | | |

BIOMARKERS TISSUE DEFICITS AND THERAPEUTIC TARGETS
FOR INTESTINAL FISTULA
Intestinal fistula nutritional deficiency 114 studies (110 malnutrition
studies), compared with: The subject composition(s) (IPI's) chemical
moiety component to detect biomarker tissue defects in the compositions
immunotherapeutic agent (IA) directed to the treatment of these biomarker
tissue defects in reestablishing tissue integrity, innate immunity, and
associated adaptive immunity and disease resolution 58 studies of efficacy
with acknowledgement of tissue deficits
1,595 studies of efficacy of the composition(s) (IPI's) chemical moiety
components (without active acknowledgement of tissue deficits) in the
composition(s) immunotherapeutic agent (IA) directed to the treatment of
these biomarker tissue defects in reestablishing tissue integrity, innate
immunity, and associated adaptive immunity and disease resolution
Precursor to Proto-plasmic Protein

| | | |
|---|---|---|
| Amino Acids | 119 | 0 |
| Essential | 6 | 0 |
| (histidine) | | |
| (isoleucine) | 0 | 0 |
| (leucine) | 14 | 1 |
| (lysine) | 5 | 0 |
| (methionine) | 6 | 0 |
| (phenylalanine) | 9 | 0 |
| (threonine) | 3 | 0 |
| (tryptophan) | 6 | 0 |
| (valine) | 1 | 0 |
| Non-essential | 9 | 0 |
| (alanine) | | |
| (arginine) | 8 | 0 |
| (asparagine) | 1 | 0 |
| (aspartic acid) | 1 | 0 |
| (cysteine) | 4 | 0 |
| (glutamic | 3 | 0 |
| acid) | | |
| (glutamine) | 8 | 0 |
| (glycine) | 21 | 0 |
| (proline) | 8 | 0 |
| (serine) | 3 | 0 |
| (tyrosine) | 6 | 0 |
| *Homocysteine | 0 | 0 |
| Lysine | 5 | 0 |
| n-acetyl cysteine | 0 | 0 |

Cell Memb. Essence & Configuration

| | | |
|---|---|---|
| Phosphatidylcholine | 32 | 0 |
| Omega 3 | 3 | 0 |
| Omega 6 | 3 | 0 |
| Omega 9 | 2 | 0 |

Extra Cellular Matrix (ECM)

| | | |
|---|---|---|
| Collagen | 67 | 1 |
| Cartilage | 7 | 0 |
| Glucosamine SO4 | 3 | 0 |
| Chondroitin SO4 | 0 | 0 |
| MSM | 0 | 0 |
| Hyaluronic Acid | 6 | 0 |
| Hyaluronan | 6 | 0 |
| Proteoglycan | 0 | 0 |
| Glycoprotein | 68 | 1 |
| Glycosaminoglycans* | 10 | 0 |
| Mucopolysaccharide* | 11 | 0 |

Micronutrients

| | | |
|---|---|---|
| Minerals | 15 | 5 |
| Vitamins* | 83 | 21 |
| Magnesium | 12 | 0 |
| Selenium | 5 | 4 |
| Vitamin C | 4 | 1 |
| Vitamin B1 | 3 | 0 |
| Potassium | 37 | 2 |
| Zinc | 16 | 8 |
| Chromium | 10 | 0 |

Adjuvants
2^{nd} Tier IPI Rx

| | | |
|---|---|---|
| Garlic | 0 | 0 |
| Resveratrol | 0 | 0 |
| Curcumin | 0 | 0 |
| Probiotics | 2 | 0 |
| Cruciferous | 0 | 0 |
| vegetables | | |
| Indole 3 | 14 | 0 |
| Albumin | 60 | 0 |
| Protein | 869 | 14 |
| Thymus | 1 | 0 |
| Nuclear Factor | 2 | |
| kappa B | | |

Quoting from the 2^{nd} Paragraph of the Letter 2001:
2. Extensively reviewed and unique to the prior art of a medical food.
3. Conforming to the Regulatory Affairs statute of a medical food. A synthetic biopharmaceutical anti-inflammatory immunotherapeutic agent using components equivalent to human tissue that also replace documented deficiencies in human tissue and the cell cycle by providing a composition that mimics human tissue from a biological and cell biology system and cell cycle vantage point without any known undesirable effects. This Colloidal & Emulsion Technology Domain, where physics, chemistry, biology, and technology meet. In the immunotherapeutic recomposition of tissue and tissue micronutrient defect concurring with the Regulatory Affairs statute of a medical food as biomarkers to activate noninvasively the patient's own stem cells and the healing system (through these molecular structures that underlie molecular embryology) all free of known undesirable effects (preapproved as safe in the Code of Federal Regulations 21—FRC21 and GRAS Regulations). A plurality of the 20 alpha amino acids (9 essential, 11 nonessential) specified by the genetic code of human and mammalian tissue wherein no more than 10% of the amino acids are in D-form have been used here in bringing about disease resolution in averting the need for surgical care for hip prosthesis, and organ and skin transplantation.
4. This second letter is presented of Jan. 12, 2011 is representative of a continuation of the last paragraph of the 2^{nd} letter where the subject composition medicament becomes a drug in vivo (2001, third paragraph and therefore designated as a drug in this letter) the tissues which we have documented oral administration as well as the transdermal immunotherapeutics IA showing this biopharmaceutical in the recomposition of tissue in providing of disease resolution in reactivation of the patient's own healing system and stem cells noninvasively without the use of foreign cells thereby reversing the deactivation of patient's healing and system stem cells by disease. When administered transdermally with the advantage of avoiding dilution by systemic use and processing by the liver as a first pass thereby avoiding modification of subject composition in meeting compositional deficiencies therapeutically.

### (See FIG. 24).

### Example Group 18

Instrumentation is used to examine living tissue non-invasively where cancer is suspected as in the above cancer examples to avoid surgical biopsy and the risk of dissemination of metastasis, the major risk of cancer in terms of longevity its spread through the body. Infected tissue could be examined in a similar fashion, as its spread is of concern there also, such as in an abscess and question of septicemia. It is also used to track biomarkers and to track immunotherapeutic agent repair of tissue by recomposition with these components of the chemical moiety.

Avoiding necessity for surgical biopsy and therefore minimizing any risk of surgical biopsy, as well as making it more economic. But most importantly to avoid surgical examination where surgery cold impose the risk of angiogenesis incorporating the freed cancer cells into the capillaries with the imminent risk of metastasis, the most feared and deadly risk of cancer. Several publications document this risk, several publications to follow.

A device wherein a non-invasive probe is utilized to obtain birefringence light signals and attached to examination probes inclusive of any endoscopy inclusive of ultrasound and ultrasound endoscopy, as well as CAT scan and MRI probe. This device for detecting mutagenic changes in a tissue, said device comprising: a polarizing light probe for subjecting a tissue sample to polarizing light and obtaining the associated birefringence luminescence signals; a polarizing fiber optic cable operably connected to the polarizing light probe; a digital display device operably connected to the polarizing fiber optic cable for observation of the birefringence light signals.

The device wherein the polarizing light probe and endoscopy avoids transmission of heat to examine living tissue at body temperature 37 degrees centigrade.

The device further comprising software capable of analyzing the birefringence light signals transmitted to the digital display device.

The device wherein the polarizing light penetrates the tissue sample to a depth of least 100 microns with adequate lighting of 500-1500 Watt bulb, air-cooled, to view living tissue without surgical biopsy, to avoid eating of tissue this light is transmitted first to the fiber-optic cable which can tolerate the heat, whereas the light but not the heat is transmitted to the living tissue.

The device adaptable to clinical setting visualization requirements such as endoscopy, surgery, surgical subspecialties such as neurosurgery.

The device wherein the adaptation includes use with a dissecting microscope or inverted microscope to give adequate room for living tissue examination.

The device for use in assessing wound or disease biomarkers of health or disease tissue.

The device further comprising use to assess tissue deficiency states.

The device further comprising a use to assess tissue deficiency states and associated biomarkers.

### Embodiments of the Invention

1. (a) a synthetic biopharmaceutical anti-inflammatory immunotherapeutic agent composition components equivalent to human tissue that also replace documented deficiencies in human tissue and the cell cycle by providing a composition that mimics human tissue from a biological and cell biology system and cell cycle vantage point without any known undesirable effects. This Colloidal & Emulsion Technology Domain, where physics, chemistry, biology, and technology meet and comprise.

In the immunotherapeutic recomposition of tissue and tissue micronutrient defect as biomarkers to activate noninvasively the patient's own stem cells and the healing system (through these molecular structures that underlie molecular embryology) all free of known undesirable effects (preapproved as safe in the Code of Federal Regulations 21—FRC21 and GRAS Regulations). A plurality of the 20 alpha amino acids (9 essential, 11 nonessential) specified by the genetic code of human and mammalian tissue wherein no more than 10% of the amino acids are in D-form have been used here in bringing about disease resolution in averting the need for surgical care for hip prosthesis, and organ and skin transplantation.

(b) further to embodiment 1, comprising of the lightest 26 elements by atomic mass of the periodic table.

(c) further comprising this anabolic medicament for treating a damaged tissue, the medicament comprising: a) at least one extracellular matrix compound in an amount effective in the damaged tissue as anti-inflammatory and anti-neoangiogenetic agent, wherein said extracellular matrix compound is selected from the group consisting of a glycosaminoglycan, a collagen, cartilage, chondroitin sulfate, heparan sulfate, dermatan sulfate, hyaluronic acid, a glycoprotein, and a proteoglycan; b) at least one polar surface active lipid, wherein said polar surface active lipid is selected from the group consisting of a phospholipid, a glycolipid and a lipoprotein; and c) a plurality of amino acids having an alpha carbon and wherein no more than 10% of the amino acids are in D-form. A plurality of the 20 alpha amino acids (9 essential, 11 nonessential) specified by the genetic code of human and mammalian tissue have been used here in bringing about disease resolution in further providing preoperative, perioperative, and postoperative care for potential surgical care as well as the opportunity for averting the need for specific surgical care.

2. The medicament according to embodiment 1, wherein said extracellular matrix compound is synthetically produced.

3. The medicament according to embodiment 1, wherein said extracellular matrix compound is obtained from a cellular or tissue source.

4. The medicament according to embodiment 3, wherein said cellular or tissue source is selected from the group consisting of a cell membrane, a tissue and an organ.

5. The medicament according to embodiment 1, wherein said polar surface active lipid is obtained from a cellular or tissue source.

6. The medicament according to embodiment 5, wherein said cellular or tissue source is selected from the group consisting of a cell membrane, a tissue, and an organ.

7. The medicament according to embodiment 5, wherein at least one extracellular matrix compound, at least one polar surface active lipid, and at least one amino acid associate through a molecular bonding force.

8. The medicament according to embodiment 7, wherein said molecular bonding force is selected from the group consisting of hydrogen bonding focus of hydrophilicity and hydrophilic surfactants, van der Waals force, and ionic interaction.

9. The medicament according to embodiment 5 further comprising at least one of (a) a mineral; (b) a vitamin; (c) an antioxidant; (d) an omega-3 oil; (e) zinc; (f) zinc oxide; (g) Vitamin A; (h) chondroitin sulfate; (i) cartilage; and (j) collagen.

10. The medicament according to embodiment 1, wherein said polar surface active lipid is synthetically produced.

11. The medicament according to embodiment 1, wherein said plurality of amino acids are obtained from a cellular or tissue source.

12. The medicament according to embodiment 11, wherein said cellular or tissue source is selected from the group consisting of a cell membrane, a tissue, and an organ.

13. The medicament according to embodiment 1, further comprising a sterile vehicle.

14. The medicament according to embodiment 1 further comprising at least one of (a) a mineral; (b) a vitamin; (c) an antioxidant; (d) an omega-3 oil as in fish oil and seed oil as alpha linolenic acid; (e) zinc; (f) zinc oxide; (g) Vitamin A; (h) chondroitin sulfate; (i) cartilage; and (j) collagen.

15. The medicament according to embodiment 1, further comprising gamma amino butyric acid or L-carnitine.

16. The medicament according to claim 1, further comprising GRAS items as Resveratrol, garlic, curcumin, indole-3 carbinol, and soy extract.

### Device Instrumentation Claims

1. A device wherein a non-invasive probe is utilized to obtain birefringence light signals and attached to examination probes inclusive of any endoscopy inclusive of ultrasound and ultrasound endoscopy, as well as CAT scan and MRI probe. This device for detecting mutagenic changes in a tissue, said device comprising:
a polarizing light probe for subjecting a tissue sample to polarizing light and obtaining the associated birefringence luminescence signals;
a polarizing fiber optic cable operably connected to the polarizing light probe;
a digital display device operably connected to the polarizing fiber optic cable for observation of the birefringence light signals.

2. The device, according to embodiment 1, wherein the polarizing light probe and endoscopy avoids transmission of heat to examine living tissue at body temperature 37 degrees centigrade.

3. The device, according to embodiment 1, further comprising software capable of analyzing the birefringence light signals transmitted to the digital display device.

4. The device, according to embodiment 1, wherein the polarizing light penetrates the tissue sample to a depth of least 100 microns with adequate lighting of 500-1500 Watt bulb, air-cooled, to view living tissue without surgical biopsy, to avoid eating of tissue this light is transmitted first to the fiber-optic cable which can tolerate the heat, whereas the light but not the heat is transmitted to the living tissue.

5. The device, according to embodiment 1, adaptable to clinical setting visualization requirements such as endoscopy, surgery, surgical subspecialties such as neurosurgery

6. The device, according to embodiment 5, wherein the adaptation includes use with a dissecting microscope or inverted microscope to give adequate room for living tissue examination.

7. The device, according to embodiment 1, for use in assessing wound or disease biomarkers of health or disease tissue.

8. The device, according to embodiment 7, further comprising use to assess tissue deficiency states.

9. The device, according to embodiment 18, further comprising a use to assess tissue deficiency states and associated biomarkers.

10. The genetic pattern of the general principle of healing and the enhancement of the expression of the genetic templating profile and healing pattern of the genome can be seen in the 2001 study done by the Whitehead Institute. This was confirmed by 2006 NIH study that shows a 77%* genetic conformity to wound healing pattern even with the abnormality cancer. The World Health Organization's 2010 article acknowledges palliative care as part of cancer treatment. The 1996 Japanese study also shows that the same results occurred in the patients' longevity with chemotherapy as with palliative care. This subject composition immunotherapeutics is representative of a highly specific palliative care treatment as well as a noninvasive, thereby palliative modality, of tissue examination.

I claim:

1. A birefringence light detecting device comprising:
a polarizing light probe for subjecting a tissue sample to polarizing light and obtaining the associated birefringence luminescence signals;
a fiber optic cable operably connected to the polarizing light probe; and
a digital display device operably connected to the polarizing fiber optic cable for observation of the birefringence light signals.

2. The device, according to claim 1, operably connected to a probe.

3. The device, according to claim 2, wherein the probe is an endoscopy probe, an ultrasound probe, a CAT scan probe or an MRI probe.

4. The device, according to claim 3, wherein the polarizing light probe is shielded to prevent transmission of heat to surrounding tissues.

5. The device, according to claim 1, further comprising software capable of analyzing the birefringence light signals transmitted to the digital display device.

6. The device, according to claim 1, wherein the polarizing light penetrates the tissue sample to a depth of least 100 microns.

7. The device, according to claim 1, adaptable to clinical setting visualization requirements such as endoscopy, surgery, surgical subspecialties such as neurosurgery

8. The device, according to claim 1, wherein the digital display device is a dissecting microscope or an inverted microscope.

9. A method for diagnosing damaged or diseased tissue biomarkers utilizing a birefringence light detecting device comprising:
a polarizing light probe for subjecting a tissue sample to polarizing light such that the tissue biomarkers are observable as emitted birefringence luminescence signals;
a fiber optic cable operably connected to the polarizing light probe for transmitting the birefringence luminescence signals emitted by the tissue sample; and
a digital display device operably connected to the polarizing fiber optic cable for receiving and displaying the birefringence luminescence signals transmitted by the polarizing fiber optic cable,
wherein said method comprises,
directing the polarizing light at a tissue to be examined;
transmitting birefringence luminescence signals via the fiber optic cable to the digital display device;
observing one or more tissue biomarkers made visible by the birefringence luminescence signal on the digital display device;
analyzing the tissue biomarkers for indications of disease or damage to the tissue.

10. The method, according to claim 9, wherein damage to tissue includes tissue deficiency states.

11. A synthetic biopharmaceutical anti-inflammatory immunotherapeutic agent composition having components equivalent to human tissue, comprising:
(i) a plurality of the 20 alpha amino acids (9 essential, 11 nonessential) specified by the genetic code of human and mammalian tissue wherein no more than 10% of the amino acids are in D-form;
(ii) at least one of the 24 elements indicated in the periodic table as components of human and mammalian tissue;
(iii) at least one extracellular matrix compound in an amount effective in the damaged tissue as anti-inflammatory and anti-neoangiogenetic agent, wherein said extracellular matrix compound is selected from the group consisting of a glycosaminoglycan, a collagen, cartilage, chondroitin sulfate, heparan sulfate, dermatan sulfate, hyaluronic acid, a glycoprotein, and a proteoglycan;
(iv) at least one polar surface active lipid, wherein said polar surface active lipid is selected from the group consisting of a phospholipid, a glycolipid and a lipoprotein.

12. The composition according to claim 11, wherein said extracellular matrix compound is synthetically produced.

13. The composition according to claim 11, wherein said extracellular matrix compound is obtained from a cellular or tissue source.

14. The composition according to claim 13, wherein said cellular or tissue source is selected from the group consisting of a cell membrane, a tissue and an organ.

15. The composition according to claim 11, wherein said polar surface active lipid is obtained from a cellular or tissue source.

16. The composition according to claim 15, wherein said cellular or tissue source is selected from the group consisting of a cell membrane, a tissue, and an organ.

17. The composition according to claim 15, wherein at least one extracellular matrix compound, at least one polar surface active lipid, and at least one amino acid associate through a molecular bonding force.

18. The composition according to claim 17, wherein said molecular bonding force is selected from the group consisting of hydrogen bonding focus of hydrophilicity and hydrophilic surfactants, van der Waals force, and ionic interaction.

19. The composition according to claim 15 further comprising at least one of (a) a mineral; (b) a vitamin; (c) an antioxidant; (d) an omega-3 oil; (e) zinc; (f) zinc oxide; (g) Vitamin A; (h) chondroitin sulfate; (i) cartilage; and (j) collagen.

20. The composition according to claim 11, wherein said polar surface active lipid is synthetically produced.

21. The composition according to claim 11, wherein said plurality of amino acids are obtained from a cellular or tissue source.

22. The composition according to claim 21, wherein said cellular or tissue source is selected from the group consisting of a cell membrane, a tissue, and an organ.

23. The composition according to claim 11, further comprising a sterile vehicle.

24. The composition according to claim 11 further comprising at least one of (a) a mineral; (b) a vitamin; (c) an antioxidant; (d) an omega-3 oil as in fish oil and seed oil as alpha linolenic acid; (e) zinc; (f) zinc oxide; (g) Vitamin A; (h) chondroitin sulfate; (i) cartilage; and (j) collagen.

25. The composition according to claim 11, further comprising gamma amino butyric acid or L-carnitine.
